(19) United States
(12) Patent Application Publication
(10) Pub. No.: US 2022/0389457 A1
(43) Pub. Date: Dec. 8, 2022

(54) COMPOSITIONS FOR DRG-SPECIFIC REDUCTION OF TRANSGENE EXPRESSION

(71) Applicant: The Trustees of the University of Pennsylvania, Philadelphia, PA (US)

(72) Inventors: James M. Wilson, Philadelphia, PA (US); Juliette Hordeaux, Philadelphia, PA (US)

(21) Appl. No.: 17/770,137

(22) PCT Filed: Oct. 22, 2020
(86) PCT No.: PCT/US2020/056881

§ 371 Date: Apr. 19, 2022

Related U.S. Application Data
(63) Continuation-in-part of application No. PCT/US2019/067872, filed on Dec. 20, 2019.
(60) Provisional application No. 63/043,600, filed on Jun. 24, 2020.
(60) Provisional application No. 63/038,514, filed on Jun. 12, 2020.
(60) Provisional application No. 63/023,602, filed on May 12, 2020.
(60) Provisional application No. 63/005,894, filed on Apr. 6, 2020.
(60) Provisional application No. 62/972,404, filed on Feb. 10, 2020.
(60) Provisional application No. 62/934,915, filed on Nov. 13, 2019.
(60) Provisional application No. 62/924,970, filed on Oct. 23, 2019.

(51) Int. Cl. C12N 15/861; C12N 9/24

(52) U.S. Cl. CPC C12N 15/861; C12N 9/2402; C12Y 302/01076; C12N 2750/14143

(57) ABSTRACT

Provided herein are nucleic acid sequence encoding hIDUA and expression cassettes containing these coding sequences. Also provided are vectors, such as recombinant adeno-associated virus (rAAV) vectors having a vector genome including a hIDUA coding sequence operably linked regulatory sequences that direct expression of the hIDUA. Also provided are compositions containing these expression cassettes and rAAV vectors and methods of treating MPS1 or an associated syndrome such as Hurler, Hurler-Scheie and/or Scheie syndrome. The compositions and methods provided are further designed to selectively repress expression of hIDUA in dorsal root ganglia.

### BACKGROUND OF THE INVENTION

The vector platform of choice for in vivo gene therapy is based on primate-derived adeno-associated viruses (AAV). In the 1960s, gene-therapy products were derived from AAVs isolated from preparations of adenoviruses (Hoggan, M. D. et al. Proc Natl Acad Sci USA 55:1467-1474, 1966). Although these vectors were safe, many programs failed in the clinic because of poor transduction. At the turn of the century, researchers discovered a family of endogenous AAVs that, as vectors, achieved much higher transduction efficiencies while retaining favorable safety profiles (Gao, G., et al. J Virol 78:6381-6388, 2004).

Untoward responses of the host to AAV vectors have been minimal. In contrast to non-viral and adenoviral vectors, which elicit vibrant acute inflammatory responses (Raper, S. E., et al. Mol Genet Metab 80:148-158, 2003; Zhang, Y., et al. Mol Ther 3:697-707, 2001), AAV vectors are not pro-inflammatory. Destructive adaptive immune responses to vector-transduced cells—such as cytotoxic T cells—have been minimal following AAV vector administration. There is evidence in animals and humans that AAV can induce tolerance to capsid or transgene products under certain circumstances depending on the serotype, dose, route of administration, and immune-suppression regimen (Gernoux, G., et al. Hum Gene Ther 28:338-349, 2017; Mays, L. E. & Wilson, J. M. Mol Ther 19:16-27, 2011; Manno, C. S., et al. Nat Med 12:342-347, 2006; Mingozzi, F., et al. Blood 110:2334-2341, 2007). However, it has been apparent amid the current explosion of clinical applications of AAV gene therapy that toxicities can limit applications of this technology.

The most severe toxicities have occurred following intravenous administration of high doses of AAV to target the CNS and musculoskeletal system. Studies in nonhuman primates (NHPs) showed the acute development of thrombocytopenia and transaminitis, which, in some cases, evolved into a lethal syndrome of hemorrhage and shock (Hordeaux, J., et al. Mol Ther 26:664-668, 2018; Hinderer, C., et al. Hum Gene Ther. 29(3):285-298, 2018). Acute elevations in liver enzymes and/or reductions in platelets have also been observed in most high-dose AAV clinical trials (AveXis, I. ZOLGENSMA Prescribing Information, 2019; Solid Biosciences Provides SGT-001 Program Update, 2019; Pfizer, Pfizer Presents Initial Clinical Data on Phase 1b Gene Therapy Study for Duchenne Muscular Dystrophy (DMD), 2019; Flanigan, K. T. et al. Molecular Genetics and Metabolism 126:S54, 2019). Although infrequent, severe toxicities are characterized by anemia, renal failure, and complement activation (Solid Biosciences, 2019; Pfizer, 2019).

More recently, the problem of degenerating neurons in the dorsal root ganglia (DRG) of NHPs and pigs that received AAV vector either into the cerebral spinal fluid (CSF) or at high doses into the blood has been observed (Hinderer, C., et al. Hum Gene Ther. 29(3):285-298, 2018; Hordeaux, J., et al. Mol Ther Methods Clin Dev 10:68-78, 2018; Hordeaux, J., et al. Mol Ther Methods Clin Dev 10:79-88, 2018). This neuronal toxicity is associated with degeneration of both the peripheral axons in peripheral nerves and the central axons that ascend through the dorsal columns of the spinal cord.

The mucopolysaccharidoses are a group of inherited disorders caused by a deficiency in specific lysosomal enzymes involved in the degradation of glycosaminoglycans (GAG), also called mucopolysaccharides. The accumulation of partially-degraded GAG interferes with cell, tissue, and organ function. Over time, the GAG accumulates within cells, blood, and connective tissue, resulting in increasing cellular and organ damage. One of the most serious of the mucopolysaccharidosis (MPS) disorders, MPS I, is caused by a deficiency of the enzyme alpha-L-iduronidase (IDUA). Specifically, IDUA is reported to remove terminal iduronic acid residues from two GAGs called heparan sulfate and dermatan sulfate. IDUA is located in lysosomes, compartments within cells that digest and recycle different types of molecules. More than 100 mutations in the IDUA gene have been found to cause mucopolysaccharidosis type I (MPS I), with single nucleotide polymorphisms (SNPs) being the most common.

A need in the art exists for gene therapy compositions and methods to safely and effectively treat patients diagnosed with MPSI.

### SUMMARY OF THE INVENTION

In one aspect, provided is a recombinant AAV (rAAV) comprising an AAV capsid having packaged therein a vector genome, wherein the vector genome comprises a coding sequence for a functional human alpha-L-iduronidase (hIDUA) and regulatory sequences which direct expression of the hIDUA in a cell, wherein the coding sequence comprises nucleotides 82 to 1959 of SEQ ID NO: 22, or a sequence at least 95% identical thereto, nucleotides 82 to 1959 of SEQ ID NO: 23, or a sequence at least 95% identical thereto, nucleotides 82 to 1959 of SEQ ID NO: 24, or a sequence at least 95% identical thereto, nucleotides 82 to 1959 of SEQ ID NO: 25, or a sequence at least 95% identical thereto, or nucleotides 82 to 1959 of SEQ ID NO: 26, or a sequence at least 95% identical thereto. In certain embodiments, the rAAV comprises a coding sequence for a functional hIDUA that comprises at least amino acids 28 to 653 of SEQ ID NO: 21, or a sequence at least 95% identical thereto. In certain embodiments, the hIDUA comprises the native signal peptide. In yet a further embodiment, the hIDUA comprises the full-length (amino acids 1 to 653) of SEQ ID NO: 21, or a sequence at least 95% identical thereto. In certain embodiments, the, hIDUA coding sequence comprises nucleotides 1 to 1959 of SEQ ID NO: 22, or a sequence at least 95% identical thereto, nucleotides 1 to 1959 of SEQ ID NO: 23, or a sequence at least 95% identical thereto, nucleotides 1 to 1959 of SEQ ID NO: 24, or a sequence at least 95% identical thereto, nucleotides 1 to 1959 of SEQ ID NO: 25, or a sequence at least 95% identical thereto, or nucleotides 1 to 1959 of SEQ ID NO: 26, or a sequence at least 95% identical thereto. In certain embodiments, the hIDUA comprises a heterologous signal peptide. In certain embodiments, the vector genome comprises a tissue-specific promoter. In certain embodiments, the vector genome comprises at least one dorsal root ganglion (drg)-specific miRNA target sequence specific for at least one of miR-183, miR-182, or miR-96, the at least one target sequence being operably linked to the 3′ end of the hIDUA coding sequence. In certain embodiments, the miRNA target sequence is selected from SEQ ID NO: 1, 2, 3, and 4. In certain embodiments, the vector genome further comprises at two, at least three, or at least four drg-specific miRNA target sequences. In certain embodiments, the rAAV provided has an AAV9, AAVhu68, or AAVrh91 capsid.

In another aspect, provided is an expression cassette comprising a nucleic acid sequence encoding a functional human alpha-galactosidase A (hIDUA) and regulatory sequences that direct expression of the hIDUA in a cell containing the expression cassette, wherein coding sequence comprises nucleotides 82 to 1959 of SEQ ID NO: 22, or a sequence at least 95% identical thereto, nucleotides 82 to 1959 of SEQ ID NO: 23, or a sequence at least 95% identical thereto, nucleotides 82 to 1959 of SEQ ID NO: 24, or a sequence at least 95% identical thereto, nucleotides 82 to 1959 of SEQ ID NO: 25, or a sequence at least 95% identical thereto; or nucleotides 82 to 1959 of SEQ ID NO: 26, or a sequence at least 95% identical thereto. In certain embodiments, hIDUA comprises a coding sequence for a functional hIDUA having at least amino acids 28 to 653 of SEQ ID NO: 21, or a sequence at least 95% identical thereto. In certain embodiments, the hIDUA comprises the native signal peptide. In certain embodiments, the hIDUA comprises the full-length (amino acids 1 to 653) of SEQ ID NO: 21, or a sequence at least 95% identical thereto. In a further embodiments, the expression cassette comprises an hIDUA coding sequence comprising nucleotides 1 to 1959 of SEQ ID NO: 22, or a sequence at least 95% identical thereto, nucleotides 1 to 1959 of SEQ ID NO: 23, or a sequence at least 95% identical thereto, nucleotides 1 to 1959 of SEQ ID NO: 24, or a sequence at least 95% identical thereto, nucleotides 1 to 1959 of SEQ ID NO: 25, or a sequence at least 95% identical thereto, or nucleotides 1 to 1959 of SEQ ID NO: 26, or a sequence at least 95% identical thereto. In yet another embodiment, the hIDUA comprises a heterologous signal peptide. In certain embodiments, the expression cassette comprises a tissue-specific promoter. In certain embodiments, the expression cassette comprises at least one dorsal root ganglion (drg)-specific miRNA target sequence specific for at least one of miR-183, miR-182, or miR-96, the at least one target sequence being operably linked to the 3′ end of the hIDUA coding sequence. In certain embodiments, the miRNA target sequence is selected from SEQ ID NO: 1, 2, 3, and 4. In certain embodiments, the expression cassette further comprises at two, at least three, or at least four drg-specific miRNA target sequences. In certain embodiments, the expression cassette is carried by a non-viral vector or a viral vector. In certain embodiments, wherein non-viral vector is selected from naked DNA, naked RNA, an inorganic particle, a lipid particle, a polymer-based vector, or a chitosan-based formulation. In certain embodiments, the vector is a recombinant parvovirus, a recombinant lentivirus, a recombinant retrovirus, a recombinant adenovirus.

In one aspect, provided is a recombinant nucleic acid comprising a sequence encoding a functional hIDUA, wherein the coding sequence comprises nucleotides 82 to 1959 of SEQ ID NO: 22, 23, 24, 25, or 26, or a sequence at least 95% identical thereto. In certain embodiments, the nucleic acid comprises a sequence encoding a functional hIDUA, wherein the coding sequence comprises nucleotides 1 to 1959 of SEQ ID NO: 22, 23, 24, 25, or 26, or a sequence at least 95% identical thereto. In a further embodiment, the recombinant nucleic acid is a plasmid.

In another aspect, provided is a host cell containing a rAAV, an expression cassette, or a recombinant nucleic acid as provided herein.

In another aspect, provided is a pharmaceutical composition comprising a rAAV, an expression cassette, or a recombinant nucleic acid as provided herein, and a pharmaceutically-acceptable carrier.

Also provided in another aspect is a method of treating a subject diagnosed with mucopolysaccharidosis type I (MPS I), wherein the method comprises administering to the subject a pharmaceutical composition provided herein. In certain embodiments, the subject has been diagnosed with Hurler syndrome, Hurler-Scheie syndrome, and/or Scheie syndrome. Also provided are uses of a rAAV, an expression cassette, a recombinant nucleic acid, or a pharmaceutical composition as provided herein to treat a subject diagnosed with MPS I, Hurler syndrome, Hurler-Scheie syndrome, and/or Scheie syndrome.

### BRIEF DESCRIPTION OF THE DRAWINGS

FIG. 1A-FIG. 1C show DRG toxicity and secondary axonopathy after AAV ICM administration. (FIG. 1A) DRG contain the cell bodies of sensory pseudo-unipolar neurons, which relay sensory messages from the periphery to the CNS through peripheral axons located in peripheral nerves and central axons located in the ascending dorsal white matter tracts of the spinal cord. (FIG. 1B) Axonopathy and DRG neuronal degeneration. Axonopathy (upper left) manifests as clear vacuoles that are either empty or filled with macrophages and cellular debris (arrow). DRG lesions (upper right and lower left): arrow shows neuronal cell-body degeneration whereas circle indicates mononuclear cell infiltration. Lower right picture shows immunostaining for the transgene encoded by AAV (green fluorescent protein (GFP) in this case). (FIG. 1C) Examples of grade 1 to grade 5 DRG lesion and grade 1 to grade 4 dorsal spinal cord axonopathy, as well as a section within normal limits (WNL). Severity grades are defined as follows: 1 minimal (<10%), 2 mild (10-25%), 3 moderate (25-50%), 4 marked (50-95%), and 5 severe (>95%). Grade 5 was never observed in spinal cord. Arrows and circles delineate neuronal degeneration with mononuclear cell infiltrates in DRG (left column) and axonopathy (right column).

FIG. 2A-FIG. 2F show high-magnification images of DRG toxicity and secondary axonopathy in the dorsal white matter tracts of the spinal cord after AAV ICM administration. (FIG. 2A) Early lesion, neuronal cell bodies (circles) are surrounded with proliferating satellite cells along with microglial cells (neuronophagia) and infiltrating mononuclear cells. (FIG. 2C) As lesions progress, neuronal cell bodies exhibit evidence of degeneration (circle) characterized by small irregular- or angular-shaped cells with fading or absent nuclei and cytoplasmic hypereosinophilia. (FIG. 2E) End stage, neuronal cell body degeneration (circles) along with complete obliteration (star) by satellite cells, microglial cells and mononuclear cells. (FIG. 2B, FIG. 2D, and FIG. 2F) Axonal degeneration of dorsal white matter tracts of the spinal cord with dilated myelin sheaths with (vertical arrows) and without (horizontal arrows) myelomacrophages, swollen axons (asterisks), and axonal debris (arrowheads). (Hematoxylin and eosin; 40×, Scale bar=50 μm).

FIG. 3A and FIG. 3B show overexpression-related toxicity model and mitigation strategy using DRG-specific miRNA-induced silencing. (FIG. 3A) Pseudo-unipolar sensory neuron cell bodies are located within DRG, surrounded by satellite cells and fenestrated capillaries. The peripheral axon of pseudo-unipolar sensory neurons is located in peripheral nerves and the central axon is located in the dorsal tracts of the spinal cord. AAV vectors hijack and overload the transcription and protein-synthesis machinery, thus leading to cellular stress—such as endoplasmic reticulum (ER) stress for secreted proteins—and secondary failure to maintain distal axons. Satellite cells undergo reactive proliferation and secrete cytokines, thereby attracting inflammatory cells such as lymphocytes. Those reversible changes can culminate in cell death. Subsequently, glial cells and macrophages infiltrate and phagocytose the neuronal cell bodies. (FIG. 3B) AAV expression cassette design for DRG-specific silencing. Four short tandem repeats of a DRG-specific miRNA reverse-complimentary sequence (miR targets) are introduced between the stop codon and the poly-A. In DRG neurons, exact base pairing between DRG-specific miRNA (such as miRNA183) and its targets in the 3′ untranslated region of the mRNA recruits the RNA-induced silencing complex (RISC), which in turn leads to silencing through mRNA cleavage. In other cell types that do not express miRNA183, translation and protein synthesis occur without any impact from the 3′ UTR region.

FIG. 4A and FIG. 4B show measurement of miR-183 abundance by qRT-PCR. (FIG. 4A) Tissues were from NHP Rhesus monkeys either naïve (not treated with AAV) or treated with vectors that did not include miR targets. n=3 for frontal cortex (Cortex), heart, spleen, cerebellum, liver, medulla and spinal cord (SC). n=2 for quadriceps (Quads) and DRG-cervical segments. miR-183 expression data are presented as the fold change compared with the Cortex. SD was calculated from biological replicates. 1-way ANOVA followed by Tukey's multiple comparison test. *p<0.05, miR183 expression in DRG compared with other tissues. (FIG. 4B) miR-183 expression in human SC and DRG from a 25-year-old male Caucasian organ donor with no history of neuropathic pain. Data presented as fold change compared with the SC. SD was calculated from triplicates of qRT-PCR wells.

FIG. 5A-FIG. 5D show miR183 targets specifically silence transgene expression in vitro and in mice DRG neurons. (FIG. 5A) GFP western blot from 293 cells co-transfected with GFP-expressing plasmids harboring miR183 or miR145 targets, and control or miR183-expression plasmids. Experiments were performed in triplicate. Data shown as mean; error bars indicate standard deviation. (FIG. 5B) DRG GFP positive neurons by IHC quantified on sections from C57BL6/J mice injected IV with AAV9.GFP control vector or AAV9.GFP-miR vectors at the dose of 4×10¹² GC (n=3 to 4 mice per group). Three DRG-enriched miR: miR183, miR145, and miR182 were screened. Data points represent average percentage of GFP-expressing neurons over total DRG neurons per mouse. Data shown as mean; error bars indicate standard deviation. Wilcoxon test, * p<0.05, ** p<0.01, *** p<0.001. (FIG. 5C) Representative pictures of GFP immunostainings from DRG quantified in panel B. (FIG. 5D) Representative pictures of cerebellum, cortex, and liver from C57BL6/J mice injected IV with AAV-PHP.B.GFP control vector or AAV-PHP.B.GFP-miR (miR183, miR145, miR182).

FIG. 6A-FIG. 6C show GFP expression in brain and peripheral organs from mice. (FIG. 6A) GFP direct fluorescence in brain cortex (exposure time 3 s) from C57BL6/J mice injected IV with AAV-PHP.B.GFP control vector or AAV-PHP.B.GFP-miR vectors at a dose of 1×1012 GC, n=4 per group. Four DRG-enriched miR: miR183, miR182, miR96, and miR145 were initially screened. (FIG. 6B) GFP direct fluorescence in liver (exposure time 1 s), heart (exposure time 3 s), and muscle (exposure time 10 s) from C57BL6/J mice injected IV with AAV9.GFP control vector or AAV9.GFP-miR vectors at a dose of 4×1012 GC, n=3-4 per group. (FIG. 6C) Quantification of GFP direct fluorescence intensity from all mice (n=3-4 per group). 1-way ANOVA followed by Tukey's multiple comparison test. * p<0.05, ** p<0.01.

FIG. 7A-FIG. 7C show miR183 targets specifically silence GFP expression in DRG and decrease toxicity after AAVhu68.GFP ICM administration to NHP. (FIG. 7A) Representative pictures of GFP-immunostained sections of DRG, spinal cord motor neurons, cerebellum, cortex, heart, and liver from adult rhesus macaques injected ICM with 3.5×10¹³ GC of AAVhu68.GFP control vector (n=2) or AAVhu68.GFP-miR183 (n=4). (FIG. 7B) Quantification of GFP-positive cells in DRG (2-4 distinct lumbar DRG per animal, n=2-4 animals per group), spinal cord (lower motor neurons, 2-5 distinct sections per animal, n=2-4 animals per group), cerebellum, and cortex in NHP (five 20× magnification fields per region, n=2-4 animals per group). Data shown as mean; error bars indicate standard deviation Wilcoxon test; * p<0.05, ** p<0.01, *** p<0.001. (FIG. 7C) Histopathology two months post-injection shows severity grades of dorsal spinal cord axonopathy, peripheral nerve axonopathy (median, peroneal, and radial nerves), DRG neuronal degeneration, and mononuclear cell infiltration. 1 minimal (<10%), 2 mild (10-25%), 3 moderate (25-50%), 4 marked (50-95%) and 5 severe (>95%—not observed). Each bar represents one animal. 0 indicates absence of lesion.

FIG. 8A-FIG. 8D show T cell and antibody responses to hIDUA in NHP. (FIG. 8A-FIG. 8C) Interferon gamma ELISPOT responses in lymphocytes isolated from PBMC, spleen, liver, and deep cervical lymph nodes 90 days post-injection. Each animal has three values representing three overlapping peptide pools covering the hIDUA sequence). Red indicates a positive ELISPOT response defined as >55 spot-forming units per 106 lymphocytes and three times the medium negative control upon no stimulation. (FIG. 8D) anti-hIDUA antibody ELISA assay, serum dilution 1:1,000.

FIG. 9 shows concentration of cytokines/chemokines in the CSF. Samples were collected at time of vector administration (D0) and 24 hours (24 h), 21 days (D21) and 35 days (D35) post-vector administration. Heat maps showing the concentration from a Milliplex MAP kit containing the following analytes: sCD137, Eotaxin, sFasL, FGF-2, Fractalkine, Granzyme A, Granzyme B, IL-la, IL-2, IL-4, IL-6, IL-16, IL-17A, IL-17E/IL-25, IL-21, IL-22, IL-23, IL-28A, IL-31, IL-33, IP-10, MIP-3a, Perforin, and TNFβ.

FIG. 10 shows miR183 targets specifically silence hIDUA expression in DRG after AAVhu68.hIDUA ICM administration to NHP. Representative pictures of hIDUA expression by anti-hIDUA antibody immunofluorescence (DRG, first row; quantification data provided in FIG. 13A), anti-hIDUA IHC (lower motor neurons, cerebellum, cortex), and anti-IDUA ISH (DRG last row; quantification data provided in FIG. 13A). hIDUA ISH: exposure time is 200 ms for AAVhu68.hIDUA with and without steroids. Sensory neurons show massive transgene mRNA expression. Exposure time is 1 s for AAV.hIDUA-miR183. Sensory neurons have low ISH signal (mRNA) in the nucleus and cytoplasm. mRNA is visible in satellite cells that surround neurons at this higher exposure time.

FIG. 11A-FIG. 11C show miR183-mediated silencing is specific to DRG neurons and fully prevents DRG toxicity in NHP ICM-administered AAVhu68.hIDUA. (FIG. 11A) Quantification of hIDUA-positive cells in DRG (5 distinct DRG per animal, n=3 animals per group), spinal cord (lower motor neurons, 2-5 distinct sections per animal, n=3 animals per group), cerebellum, and cortex in NHP (five 20× magnification fields per region, n=3 animals per group). Data shown as mean; error bars indicate standard deviation Wilcoxon test, * p<0.05, ** p<0.01, *** p<0.001, **** p<0.0001. (FIG. 11B) Histopathology scoring three months post-injection: DRG severity grade from β-5 (plot showing scores from all the DRG—a minimum of 3 cervical, 3 thoracic, and 3 lumbar per animal); dorsal axonopathy grade from β-5 (plot showing scores from all the distinct sections—a minimum of 3 cervical, 3 thoracic, and 3 lumbar spinal cord section per animal); and median nerve score—the sum of axonopathy and fibrosis severity grades (β-10) established on 4 sections per animal (right, left proximal and distal median nerves). Severity grades defined as follows: 0 no lesion, 1 minimal (<10%), 2 mild (10-25%), 3 moderate (25-50%), 4 marked (50-95%) and 5 severe (>95%—not observed). Data shown as mean; error bars indicate standard deviation. Wilcoxon test, * p<0.05, ** p<0.01, *** p<0.001. (FIG. 11C) ISH using hIDUA transgene-specific probes, high magnification of DRG sensory neurons and satellite cells; 1 s exposure time with blue DAPI nuclear counterstain. Arrows: DRG sensory neurons; arrowheads: satellite cells.

FIG. 12 shows vector biodistribution in brain, spinal cord, and DRG in NHP. Vector genomes quantification by real-time polymerase chain reaction using Taqman reagents and primers/probes that targeted the rBG polyadenylation sequence of the vectors. Results expressed in genome copy per diploid genome. Error bars represent standard deviation (n=3 animals per group).

FIG. 13A-FIG. 13F show IHC for apoptotic marker activated caspase-3 of DRG with spleen as a positive control. (FIG. 13A and FIG. 13B) Degenerating neuronal cell bodies (circles) and surrounding cellular infiltrates (arrowheads) are positive for activated caspase-3 in animals injected with AAVhu68.eGFP and AAVhu68.hIDUA, respectively. (FIG. 13C) An animal injected with AAVhu68.eGFP.miR183 shows rare positive caspase-3 immunostaining in degenerating neuronal cell bodies (circles); Inset: The majority of DRG sections from animals injected with AAVhu68.eGFP.miR183 are negative for activated caspase-3. (FIG. 13D) Neurons from animals injected with AAVhu68.hIDUA.miR183 are also negative for activated caspase-3. (FIG. 13E) The neuronal cell bodies of a naive, non-AAV-injected control NHP with normal DRG are diffusely light brown and considered negative, consistent with background staining. (FIG. 13F) Spleen, as positive control, from an AAVhu68-injected NHP has a strongly positive, multifocal signal for activated caspase-3 in cellular debris of the germinal center and a multifocal positive signal within leukocytes of the red pulp (arrows). The surrounding white and red pulp is diffusely light brown, consistent with background staining. Activated caspase-3 IHC; 20×, Scale bar=100 μm.

FIG. 14A-FIG. 14E shows IHC for UPR-regulated ATF6 in DRG. (FIG. 14A) Degenerating neuronal cell bodies (circles) in an animal injected with AAVhu68.eGFP are lightly positive for ATF6; satellite cells surrounding the majority of neuronal cell bodies (vertical arrow), most prominently those clusters lacking neuronal cell bodies (horizontal arrows), are strongly ATF6-positive. (FIG. 14B) Degenerating neuronal cell bodies (circles) from an animal injected with AAVhu68.hIDUA is negative for ATF6 in degenerating neurons; satellite cells are strongly positive in the cytoplasm (horizontal arrows). (FIG. 14C) Satellite cells in clusters lacking neuronal cell bodies (horizontal arrows) in an animal injected with AAVhu68.eGFP.miR183 are positive for ATF6; the degenerating neuronal cell bodies (circle) are negative. The majority of DRG sections from the animal injected with AAVhu68.eGFP.miR183 are negative for ATF6 (inset). (FIG. 14D) Neuronal cell bodies and satellite cells from an animal injected with AAVhu68.hIDUA.miR183 are negative for ATF6. (FIG. 14E) The neuronal cell bodies of a naive, non-AAV-injected control NHP with normal DRG are also negative for ATF6. ATF6 IHC; 20×, Scale bar=100 μm.

FIG. 15A-FIG. 15E show IHC for extrinsic apoptotic marker activated caspase-8 in DRG. Degenerating neuronal cell bodies (circles) are caspase 8-negative in animals injected with AAVhu68.eGFP (FIG. 15A), AAVhu68.hIDUA (FIG. 15B), and AAVhu68.eGFP.miR183 (FIG. 15C). The surrounding cellular infiltrate is strongly positive (arrows). (FIG. 15D) Neurons from an animal injected with AAVhu68.hIDUA.miR183 are caspase 8-negative and caspase 8-positive interstitial cells are rare (arrows). (FIG. 15E) The neuronal cell bodies of a naive, non-AAV-injected control NHP with normal DRG are caspase 8-negative with caspase 8-positive interstitial cells are rare (arrows). Activated caspase-8 IHC; 40×, Scale bar=50 μm.

FIG. 16A-FIG. 16F show IHC for intrinsic apoptotic marker activated caspase-9 of DRG. (FIG. 16A) Degenerating neuronal cell bodies (circle) in an animal injected with AAVhu68.eGFP are caspase-9-positive with increased positivity in cellular infiltrate (horizontal arrows). (FIG. 16B) A degenerating neuronal cell body in an animal injected with AAVhu68.hIDUA is caspase 9-negative with few caspase-9-positive cells in cellular infiltrate (horizontal arrow). (FIG. 16C) Neurons from an animal injected with AAVhu68.eGFP.miR183 are negative with positive infiltrating cells (horizontal arrow). (FIG. 16D) Neurons from an animal injected with AAVhu68.hIDUA.miR183 are negative; degenerating neuronal cell bodies are not observed (FIG. 16E) Neuronal cell bodies of naïve, non-AAV-injected control NHP with normal DRG are negative with rare positive interstitial cells (horizontal arrow). (FIG. 16F) Spleen, positive control, from AAVhu68-injected NHP is positive in cellular debris of germinal centers and leukocytes of red pulp (vertical arrows). Activated caspase-9 IHC; 40×, Scale bar=50 μM.

FIG. 17A-FIG. 17D show a comparison of IDUA activity following administration of engineered sequences encoding hIDUA. Wildtype male mice were injected IV with 1×10¹¹GC of AAVhu68 for delivery of hIDUA sequences (hIDUACoV1-SEQ ID NO: 22; hIDUACoV2-SEQ ID NO: 23; hIDUACoV3-SEQ ID NO: 24; hIDUACoV4-SEQ ID NO: 25; hIDUACoV5-SEQ ID NO: 26) or a non-optimized, native coding sequence (hIDUAnat). IDUA activity was measured in serum at days 7 and 8 (FIG. 17A) and in brain (FIG. 17B), heart (FIG. 17C), and liver (FIG. 17D) on day 7.

FIG. 18A-FIG. 18F show results following administration of AAVhu68.hIDUAcoV1 with or without miR183 target sequences (4×repeats) to mice. (FIG. 18A) MPS I mice (IDUA KO) were injected ICV with 1×10¹¹GC and sacrificed on day 30 or day 90 post injection. In the first study that used the unmodified hIDUA (vector 1), a cohort of young mice (1-2 months of age at treatment) was compared to a cohort of old mice (6-8 months of age) with advanced disease at treatment. The second study using the miR183 target-modified vector used only young 1-3 months old mice. (FIG. 18B-FIG. 18D) IDUA activity in brain and spinal cord were compared. One rostral coronal portion of the brain and the thoraco-lumbar spinal cord were flash frozen. After tissue lysis and clarification, IDUA enzyme activity was measured using an artificial substrate 4-methylumbelliferone (4-MU) based fluorescent assay. Results were normalized per mg of protein. (FIG. 18E and FIG. 18F) Tissues were processed to evaluate storage reduction using LAMP1 immunofluorescence as a marker of therapeutic efficacy.

FIG. 19A and FIG. 19B show the results from a sponge effect study involving an analysis of miR183 cluster-regulated gene expression in NHPs (AAV-IDUA vs AAV-IDUA-4×miR183). FIG. 19A provides a miR183 cluster regulated gene mRNA quantification in dorsal root ganglia (DRG). FIG. 19B provides the results in the cortex. There is no increased expression of miR183 cluster-regulated genes (CACNA2D1 or CACNA2D2), comparing results from AAV-IDUA and AAV-IDUA-miR183 animals in either DRG (high miR183 abundance) or frontal cortex (low miR183 abundance).

FIG. 20 shows results of AAV9 transduction of various vectors carrying an eGFP transgene with our without four copies of the miR183 target sequences at low (5×10⁵) or high (2.5×10⁸) concentration. The low and high dose without miR183 was tested with or without adenovirus type 5 (Ad5) helper co-transfection at a multiplicity of infection (MOI) of 100 (for low dose AAV9-eGFP) or 10 (high dose AAV9-eGFP). All DRG neurons are transduced and no visible signs of toxicity were observed. No GFP expression was observed in DRG neurons, while some expression was observed in fibroblast like cells. The results confirm repression of GFP transcription with the 4×miR183 target expression cassettes.

FIG. 21 shows the results from a sponge effect study in rat DRG cells. The data show that miR183 levels in rat DRG cells are decreased when cells are transduced with the AAV9-eGFP-mir183. AAF9-eGFP-miR183-shows target engagement on the GFP-miR183 mRNA.

FIG. 22A-FIG. 22C show the effect of the miR183 sponge effect study in rat DRG cells as assessed in three known miR183-regulated transcripts. FIG. 22A shows the results of CACANA2D1 relative expression in rat DRG cells following delivery of a mock vector, AAV-GFP, or a AAV-GFP-miR183 vector. FIG. 22B shows the results of CACANA2D2 relative expression in rat DRG cells following delivery of a mock vector, AAV-GFP, or a AAV-GFP-miR183 vector. FIG. 22C shows the results of ATF3 expression in rat DRG cells following delivery of a mock vector, AAV-GFP, or a AAV-GFP-miR183 vector. No changes in the relative expression of the mRNA levels of these three miR183 regulated transcripts were observed.

FIG. 23 shows neuroanatomy and microscopic findings. Neuronal cell bodies of the DRG (A) project axons centrally into the ascending (sensory) dorsal white matter tracts of the spinal cord (C) and into the peripheral nervous system (D). (A1-D1) Neuroanatomical relationship of the microscopic lesions associated with DRG pathology. Neuronal cell body degeneration (circles, A1) in the DRG results in axonal degeneration (vertical arrows, B1) with or without periaxonal fibrosis (horizontal arrows, B1) extending both centrally and peripherally in the nerve root. Axonal degeneration in the DRG nerve root extends centrally into the ascending dorsal white matter tracts of the spinal cord (vertical arrows, C1) and into peripheral nerves (vertical arrows, D1) with or without periaxonal fibrosis (horizontal arrows, D1). (A2-D2) Normal DRG, DRG nerve root, dorsal white matter of spinal cord and peripheral nerve. (Hematoxylin and eosin; 20×, Scale bar=100 μm). (E-H) High magnification images of varying stages of DRG pathology. (E) Early in the degenerative process, the neuronal cell bodies appear relatively normal (circles) with only proliferating satellite cells along with microglial cells and infiltrating mononuclear cells (neuronophagia). (F) As the lesions progress, the neuronal cell bodies exhibit evidence of degeneration (vertical arrow) characterized by small, irregular- or angular-shaped cells with fading or loss of nuclei and cytoplasmic hypereosinophilia. (G) Neuronal cell body degeneration (circles) can result in complete obliteration (star) by satellite cells, microglial cells and mononuclear cells; this is considered end-stage degeneration. (H) Normal DRG. (Hematoxylin and eosin; 40×, Scale bar=50 μm)

FIG. 24A-FIG. 24D show effects of study characteristics on severity of DRG pathology. Average pathology scores in DRG (black) and dorsal spinal cord (SC) axons (grey) with different (FIG. 24A) routes of administration, (FIG. 24B) vector doses, (FIG. 24C) times post-injection for tissue collection, and (FIG. 24D) study conduct compliance with GLP guidelines. Mean results with standard error of mean; tables indicate number of animals (n) and number of histological sections scored (count) in each group. The comparison between groups was done using Wilcoxon rank-sum test within each DRG and spinal cord regions (i.e., cervical, thoracic, lumbar) and the combined p-value was calculated for the overall DRG or spinal cord inter-group comparison using Fisher's method with statistical significance assessed at the 0.05 level. * indicate significance for inter-group comparison and # indicate (FIG. 24A) significance for comparison with the vehicle control group, or (FIG. 23C) significance for comparison with the 180+ day time point. *, # p<0.05; **, ## p<0.01; ***, ### p<0.001; ****, #### p<0.0001. Color code for statistics symbols: black for DRG and grey for SC.

FIG. 25A and FIG. 25B show effects of animal characteristics on severity of DRG pathology. Average pathology scores in DRG (black) and dorsal spinal cord (SC) axons (grey) with different (FIG. 25A) age of the animals at injection, and (FIG. 25B) sex of the animals (rhesus macaques only). Mean results with standard error of mean; tables indicate number of animals (n) and number of histological sections scored (count) in each group. The comparison between groups was done using Wilcoxon rank-sum test within each DRG and spinal cord regions (i.e., cervical, thoracic, lumbar) and the combined p-value was calculated for the overall DRG or spinal cord inter-group comparison using Fisher's method with statistical significance assessed at the 0.05 level. * indicate significance for inter-group comparison and # indicate (FIG. 25A) significance for comparison with the infant age group. *, # p<0.05; **, ## p<0.01; ***, ### p<0.001; ****, #### p<0.0001. Color code for statistics symbols: black for DRG and grey for SC.

FIG. 26A-FIG. 26D show effects of vector characteristics on severity of DRG pathology. Average pathology scores in DRG (black) and dorsal spinal cord (SC) axons (grey) with different (FIG. 26A) capsids, (FIG. 26B) promoters, and (FIG. 26C) transgenes, and secreted vs. non-secreted transgenes (FIG. 26D). Transgenes were arranged from 1 to 20 based on the severity of SC pathology. Mean results with standard error of mean; tables indicate number of animals (n) and number of histological sections scored (count) in each group. (FIG. 26A, FIG. 26B, and FIG. 26D). The comparison between groups was done using Wilcoxon rank-sum test within each DRG and spinal cord regions (i.e., cervical, thoracic, lumbar) and the combined p-value was calculated for the overall DRG or spinal cord inter-group comparison using Fisher's method with statistical significance assessed at the 0.05 level. * indicate significance for inter-group comparison: *p<0.05; **p<0.01; ***p<0.001; ****p<0.0001. Color code for statistics symbols: black for DRG and grey for SC. C=cervical, T=thoracic, L=lumbar regions.

FIG. 27 shows regional pathology scores with distribution of severity grades. Mean percentage proportion of pathology scores with standard error of mean (red dots and bars) and distribution of severity grades by region (stacked columns). Tables indicate number of animals (n) and number of histological sections scored (count) in each group. The comparison between means was done using Wilcoxon rank-sum test between TRG and DRG and between DRG and SC respective regions (i.e., cervical, thoracic, and lumbar). Statistical significance was assessed at the 0.05 level. * indicate significance for trigeminal nerve ganglion (TRG) to DRG comparisons; # indicate significance for DRG to SC regional comparisons. ** p<0.01; #### p<0.0001.

FIG. 28A and FIG. 28B show peripheral nerve pathology. Mean percentage proportion of pathology scores with standard error of mean (red dots and bars), and distribution of severity grades by peripheral nerve (stacked columns). Tables indicate number of animals (n) and number of histological sections scored (count) in each group. No statistical analysis was performed as some peripheral nerves were not collected in a majority of studies.

FIG. 29A-FIG. 29D show effects of study characteristics on severity of DRG pathology split by spinal region. Average pathology scores in DRG (black) and dorsal spinal cord (SC) axons (grey) regions with different (FIG. 29A) routes of administration, (FIG. 29B) vector doses, (FIG. 29C) times post-injection for tissue collection, and (FIG. 29D) study conduct compliance with GLP guidelines. Mean results with standard error of mean; tables indicate number of animals (n) and number of histological sections scored (count) in each group. C=cervical, T=thoracic, L=lumbar regions.

FIG. 30A and FIG. 30B show effects of animal characteristics on severity of DRG pathology split by spinal region. Average pathology scores in DRG (black) and dorsal spinal cord (SC) axons (grey) regions with different (FIG. 30A) age of the animals at injection, and (FIG. 30B) sex of the animals (rhesus macaques only). Mean results with standard error of mean; tables indicate number of animals (n) and number of histological sections scored (count) in each group. C=cervical, T=thoracic, L=lumbar regions.

FIG. 31A-FIG. 31C show effects of vector characteristics on severity of DRG pathology split by spinal region. Average pathology scores in DRG (black) and dorsal spinal cord (SC) axons (grey) regions with different (FIG. 31A) capsids, (FIG. 31B) promoters, and (FIG. 31C) transgenes. Transgenes were arranged from 1 to 20 based on the severity of SC pathology. Mean results with standard error of mean; tables indicate number of animals (n) and number of histological sections scored (count) in each group. C=cervical, T=thoracic, L=lumbar regions.

### DETAILED DESCRIPTION OF THE INVENTION

Expression cassettes and replication deficient adeno-associated viruses (“AAVs”) for delivery of a human alpha-L-iduronidase (hIDUA) gene to human subjects are provided herein. The recombinant AAV (“rAAV”) vector used for delivering the hIDUA gene (“rAAV.hIDUA”) has tropism for the CNS (e.g., an rAAV bearing an AAVhu68 capsid), and the hIDUA transgene is controlled by specific expression control elements (e.g., CB7, chicken β-actin promoter with cytomegalovirus enhancer elements). In certain embodiments, pharmaceutical compositions suitable for intrathecal, intracisternal, and systemic administration are provided, which comprise a suspension of expression cassettes or rAAV.hIDUA vectors in a formulation buffer comprising a physiologically compatible aqueous buffer, surfactant, and/or optional excipients.

In certain aspects, the compositions and methods provided herein are useful in therapies for delivery of a functional hIDUA where the transgene expression is repressed in DRG neurons through the inclusion of miRNA target sequences in the vector genome or expression cassette. As used herein, the terms “repressed” and “repression” include partial reduction or complete extinction or silencing of transgene expression. Transgene expression may be assessed using an assay suitable for the selected transgene. The compositions and methods provided decrease toxicity of the DRG characterized by neuronal degeneration, secondary dorsal spinal cord axonal degeneration, and/or mononuclear cell infiltrate. In certain embodiments, the expression cassette or vector genome comprises one or more miRNA target sequences in the untranslated region (UTR) 3′ to a gene product coding sequence. Suitably, two or more miRNA target sequences are provided in tandem, optionally separated by a spacer sequence. In certain embodiments, three or more miRNA target sequences are provided in tandem, optionally separated by a spacer sequence. In certain embodiments, eight miRNA sequences are provided in tandem, optionally separated by spacer sequences.

As used herein, a “therapeutically effective amount” refers to the amount of a composition (e.g. a rAAV.hIDUA composition) that delivers and expresses in the target cells an amount of enzyme sufficient to ameliorate or treat one or more of the symptoms of MPSI, and/or Hurler, and/or Hurler-Scheie and/or Scheie syndromes. “Treatment” may include preventing the worsening of the symptoms of one of the MPSI syndromes and possibly reversal of one or more of the symptoms thereof. Methods of assessing therapeutic effectiveness (efficacy) are described in detail below. A “therapeutically effective amount” for human patients may be predicted based on an animal model. Examples of a suitable feline model and a suitable canine model have been previously described. See, C. Hinderer et al, Molecular Therapy (2014); 22 12, 2018-2027; A. Bradbury, et al, Human Gene Therapy Clinical Development. March 2015, 26(1): 27-37, which are incorporated herein by reference. With respect to the canine model, the model is typically an immune suppressed animal model, or a tolerized animal, as intravenous administration in dogs has been observed to elicit a strong, sustained antibody response to human IDUA, whereas in human patients, administration is well tolerated. In these models, reversal of certain symptoms may be observed and/or prevention of progression of certain symptoms may be observed. For example, correction of corneal clouding may be observed, and/or correction of lesions in the central nervous system (CNS) is observed, and/or reversal of perivascular and/or meningeal gag storage is observed. The goal of treatment is to functionally replace the patient's defective alpha-L-iduronidase via rAAV-based CNS-directed gene therapy as a viable approach to treat disease. As expressed from the rAAV vector described herein, expression levels of at least about 2% of normal levels as detected in the CSF, serum, neurons, or other tissue or fluid, may provide therapeutic effect. However, higher expression levels may be achieved. Such expression levels may be from 2% to about 100% of normal functional human IDUA levels. In certain embodiments, higher than normal expression levels may be detected in CSF, serum, or other tissue or fluid.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199 (3): p. 381-390, which is incorporated by reference herein.

“Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention. It should be understood that while various embodiments in the specification are presented using “comprising” language, under various circumstances, a related embodiment is also described using “consisting of” or “consisting essentially of” language.

It is to be noted that the term “a” or “an”, refers to one or more, for example, “a vector”, is understood to represent one or more vector(s). As such, the terms “a” (or “an”), “one or more,” and “at least one” is used interchangeably herein. As used herein, the term “about” means a variability of plus or minus 10% from the reference given, unless otherwise specified.

human alpha-L-iduronidase (hIDUA)

As used herein, the terms “human alpha-L-iduronidase” and “hIDUA” are used interchangeably to refer to a human alpha-L-iduronidase enzyme. It will be understood that the Greek letter “alpha” and the symbol “a” are used interchangeably throughout this specification. As used herein, hIDUA refers to native (wild-type) hIDUA proteins and also variant hIDUA proteins expressed from the nucleic acid sequences provided herein, or functional fragments thereof, which restore a desired function, ameliorate symptoms, improve symptoms associated with one or more of the of MPSI, Hurler, and/or Hurler-Scheie and/or Scheie syndromes when delivered in a composition or by a method as provided herein.

The “human alpha-L-iduronidase” or “hIDUA” may be, for example, a full-length protein (including a signal peptide and the mature protein), the mature protein, a variant protein as described herein, or a functional fragment thereof. As used herein, the term “functional hIDUA” refers to an enzyme having the amino acid sequence of the full-length native (wild-type) protein (as shown in SEQ ID NO: 21 and UniProtKB accession number: P35475-1), a variant thereof (including those described herein), a mutant thereof with a conservative amino acid replacement, a fragment thereof, a full-length or a fragment of any combination of the variant and the mutant with a conservative amino acid replacement, which provides at least about 10%, at least about 20%, at least about 30%, at least about 40%, at least about 50%, at least about 60%, at least about 70%, at least about 75%, at least about 80%, at least about 90%, or about the same, or greater than 100% of the biological activity level of a native (wild-type) hIDUA. In certain embodiments, a functional hIDUA comprises the substrate binding region (amino acids 305 and 306) of the native hIDUA. Several naturally occurring functional polymorphisms (variants) of hIDUA have been described and may be encompassed within the scope of this invention. Such variants have been described; see, e.g., in WO 2014/151341, which is incorporated herein by reference, as well as, e.g., UniProtKB/Swiss-Prot; uniprot.org/uniprot/P35475, which is also incorporated by reference.

```
human alpha-L-iduronidase-
(signal peptide-amino acids 1 to 27)
(SEQ ID NO: 21)
                10 20 30
MRPLRPRAAL LALLASLLAA PPVAPAEAPH
                40 50 60
LVHVDAARAL WPLRRFWRST GFCPPLPHSQ
                70 80 90
ADQYVLSWDQ QLNLAYVGAV PHRGIKQVRT
              100 110 120
HWLLELVTTR GSTGRGLSYN FTHLDGYLDL
              130 140 150
LRENQLLPGF ELMGSASGHF TDFEDKQQVF
              160 170 180
EWKDLVSSLA RRYIGRYGLA HVSKWNFETW
              190 200 210
NEPDHHDFDN VSMTMQGFLN YYDACSEGLR
              220 230 240
AASPALRLGG PGDSFHTPPR SPLSWGLLRH
              250 260 270
CHDGTNFFTG EAGVRLDYIS LHRKGARSSI
              280 290 300
SILEQEKVVA QQIRQLFPKF ADTPIYNDEA
              310 320 330
DPLVGWSLPQ PWRADVTYAA MVVKVIAQHQ
              340 350 60
NLLLANTTSA FPYALLSNDN AFLSYHPHPF
              370 380 390
AQRTLTARFQ VNNTRPPHVQ LLRKPVLTAM
              400 410 420
GLLALLDEEQ LWAEVSQAGT VLDSNHTVGV
              430 440 450
LASAHRPQGP ADAWRAAVLI YASDDTRAHP
              460 470 480
NRSVAVTLRL RGVPPGPGLV YVTRYLDNGL
              490 500 510
CSPDGEWRRL GRPVFPTAEQ FRRMRAAEDP
              520 530 540
VAAAPRPLPA GGRLTLRPAL RLPSLLLVHV
              550 560 570
CARPEKPPGQ VTRLRALPLT QGQLVLVWSD
              580 590 600
EHVGSKCLWT YEIQFSQDGK AYTPVSRKPS
              610 620 630
TFNLFVFSPD TGAVSGSYRV RALDYWARPG
              640 650
PFSDPVPYLE VPVPRGPPSP GNP
```

Native human IDUA coding sequence (SEQ ID NO: 20) (NCBI Reference Sequence: NM 000203.5); (signal peptide—nucleotides 1 to 81)

atgcgtcccctgcgcccccgcgccgcgctgctggcg
ctcctggcctcgctcctggccgcgcccccggtggc
cccggccgaggccccgcacctggtgcatgtggacg
cggcccgcgcgctgtggcccctgcggcgcttctgg
aggagcacaggcttctgccccccgctgccacacag
ccaggctgaccagtacgtcctcagctgggaccagc
agctcaacctegcctatgtgggcgccgtccctcac
cgcggcatcaagcaggtccggacccactggctgct
ggagcttgtcaccaccagggggtccactggacggg
gcctgagctacaacttcacccacctggacgggtac
ctggaccttctcagggagaaccagctcctcccagg
gtttgagctgatgggcagcgcctcgggccacttca
ctgactttgaggacaagcagcaggtgtttgagtgg
aaggacttggtctccagcctggccaggagatacat
cggtaggtacggactggcgcatgtttccaagtgga
acttcgagacgtggaatgagccagaccaccacgac
tttgacaacgtctccatgaccatgcaaggcttcct
gaactactacgatgcctgctcggagggtctgcgcg
ccgccagccccgccctgcggctgggaggccccggc
gactccttccacaccccaccgcgatccccgctgag
ctggggcctcctgcgccactgccacgacggtacca
acttcttcactggggaggcgggcgtgcggctggac
tacatctccctccacaggaagggtgcgcgcagcte
catctccatcctggagcaggagaaggtcgtcgcgc
agcagatccggcagctcttccccaagttcgcggac
acccccatttacaacgacgaggcggacccgctggt
gggctggtccctgccacagccgtggagggcggacg
tgacctacgcggccatggtggtgaaggtcatcgcg
cagcatcagaacctgctactggccaacaccacctc
cgccttcccctacgcgctcctgagcaacgacaatg
ccttcctgagctaccacccgcaccccttcgcgcag
cgcacgctcaccgcgcgcttccaggtcaacaacac
ccgcccgccgcacgtgcagctgttgcgcaagccgg
tgctcacggccatggggctgctggcgctgctggat
gaggagcagctctgggccgaagtgtcgcaggccgg
gaccgtcctggacagcaaccacacggtgggcgtcc
tggccagcgcccaccgcccccagggcccggccgac
gcctggcgcgccgcggtgctgatctacgcgagcga
cgacacccgcgcccaccccaaccgcagcgtcgcgg
tgaccctgcggctgcgcggggtgccccccggcccg
ggcctggtctacgtcacgcgctacctggacaacgg
gctctgcagccccgacggcgagtggcggcgcctgg
gccggcccgtcttccccacggcagagcagttccgg
cgcatgcgcgcggctgaggacccggtggccgcggc
gccccgccccttacccgccggcggccgcctgaccc
tgcgccccgcgctgcggctgccgtcgcttttgctg
gtgcacgtgtgtgcgcgccccgagaagccgcccgg
gcaggtcacgcggctccgcgccctgcccctgaccc
aagggcagctggttctggtctggtcggatgaacac
gtgggctccaagtgcctgtggacatacgagatcca
gttctctcaggacggtaaggcgtacaccccggtca
gcaggaagccatcgaccttcaacctctttgtgttc
agcccagacacaggtgctgtctctggctcctaccg
agttcgagccctggactactgggcccgaccaggcc
ccttctcggaccctgtgccgtacctggaggtccct
gtgccaagagggcccccatccccgggcaatccatg
a

With reference to the numbering of the full-length native hIDUA of SEQ ID NO: 20, there is a signal peptide at amino acid positions 1 to 27 and the mature protein includes amino acid 28 to 653. As used herein, a “signal peptide” refers to a short peptide (usually about 16 to 35 amino acids) present at the N-terminus of newly synthesized proteins. A signal peptide, and in some cases the nucleic acid sequences encoding such a peptide, may also be referred to as a signal sequence, a targeting signal, a localization signal, a localization sequence, a transit peptide, a leader sequence, or a leader peptide. In certain embodiments, the hIDUA is a mature protein (lacking a signal peptide sequence).

As described herein, an hIDUA may include a native signal peptide (i.e. amino acids 1 to 27 of SEQ ID NO: 21) or, alternatively, a heterologous signal peptide. In certain embodiments, a hIDUA includes a heterologous signal peptide. In certain embodiments, such a heterologous signal peptide is preferably of human origin and may include, e.g., an IL-2 signal peptide. Particular heterologous signal peptides workable in the certain embodiments include amino acids 1-20 from chymotrypsinogen B2, the signal peptide of human alpha-1-antitrypsin, amino acids 1-25 from iduronate-2-sulphatase, and amino acids 1-23 from protease CI inhibitor. See, e.g., WO2018046774. Other signal/leader peptides may be natively found in an immunoglobulin (e.g., IgG), a cytokine (e.g., IL-2, IL12, IL18, or the like), insulin, albumin, β-glucuronidase, oncostatin, alkaline protease or the fibronectin secretory signal peptides, amongst others. See, also, e.g., signalpeptide.de/index.php?m=listspdb_mammalia. Such a chimeric hIDUA may have the heterologous leader in the place of the native signal peptide. Optionally, an N-terminal truncation of the hIDUA enzyme may lack only a portion of the signal peptide (e.g., a deletion of about 2 to about 25 amino acids, or values therebetween), the entire signal peptide, or a fragment longer than the signal peptide (e.g., up to amino acids 70 based on the numbering of SEQ ID NO: 21. Optionally, such an enzyme may contain a C-terminal truncation of about 5, 10, 15, or 20 amino acids in length.

In certain embodiments, an hIDUA may be selected which has a sequence that is at least 95% identical, at least 97% identical, or at least 99% identical to the full-length (amino acids 1 to 653) of SEQ ID NO: 21. In certain embodiments, provided is a sequence which is at least 95%, at least 97%, or at least 99% identical to the mature protein (amino acids 28 to 653) of SEQ ID NO: 21. In certain embodiments, the sequence having at least 95% to at least 99% identity to the hIDUA of either the full-length (amino acids 1 to 653) or mature protein (amino acids 32 to 653) is characterized by having an improved biological effect and better safety profile than the reference (i.e. native) hIDUA when tested in an appropriate animal model. In certain embodiments, the hIDUA enzyme contains modifications in designated positions in the hIDUA amino acid sequence.

As used herein, the “conservative amino acid replacement” or “conservative amino acid substitutions” refers to a change, replacement or substitution of an amino acid to a different amino acid with similar biochemical properties (e.g. charge, hydrophobicity and size), which is known by practitioners of the art. Also see, e.g. FRENCH et al. What is a conservative substitution? Journal of Molecular Evolution, March 1983, Volume 19, Issue 2, pp 171-175 and YAMPOLSKY et al. The Exchangeability of Amino Acids in Proteins, Genetics. 2005 August; 170(4): 1459-1472, each of which is incorporated herein by reference in its entirety.

In one aspect, provide herein are nucleic acid sequences and, for example expressions cassettes and vectors comprising the same, which encode a functional hIDUA protein. In one embodiment, the nucleic acid sequence is the wild-type coding sequence reproduced in SEQ ID NO: 20. In further embodiments, the nucleic acid sequence is at least about 60%, at least about 65%, at least about 70%, at least about 75%, at least about 80% identical to the wild type hIDUA sequence of SEQ ID NO: 20, and encodes a function hIDUA.

As used herein, “a nucleic acid” refers to a polymeric form of nucleotides and includes RNA, mRNA, cDNA, genomic DNA, peptide nucleic acid (PNA) and synthetic forms and mixed polymers of the above. A nucleotide refers to a ribonucleotide, deoxynucleotide or a modified form of either type of nucleotide (e.g., a peptide nucleic acid oligomer). The term also includes single- and double-stranded forms of DNA. The skilled person will appreciate that functional variants of these nucleic acid molecules are described herein. Functional variants are nucleic acid sequences that can be directly translated, using the standard genetic code, to provide an amino acid sequence identical to that translated from a parental nucleic acid molecule.

In certain embodiments, the nucleic acid molecules encoding a functional hIDUA, and other constructs as described herein are useful in generating expression cassettes and vector genomes and may be engineered for expression in yeast cells, insect cells, or mammalian cells, such as human cells. Methods are known and have been described previously (e.g. WO 96/09378). A sequence is considered engineered if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in www.kazusa.jp/codon. Preferably more than one non-preferred codon, preferably most or all non-preferred codons, are replaced by codons that are more preferred. Preferably the most frequently used codons in an organism are used in an engineered sequence. Replacement by preferred codons generally leads to higher expression. It will also be understood by a skilled person that numerous different nucleic acid molecules can encode the same polypeptide as a result of the degeneracy of the genetic code. It is also understood that skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g. GeneArt, GenScript, Life Technologies, Eurofins).

In certain embodiments, the nucleic acids, expression cassettes, vector genomes described herein include an hIDUA coding sequence that is an engineered sequence. In certain embodiments, the engineered sequence is useful to improve production, transcription, expression, or safety in a subject. In certain embodiments, the engineered sequence is useful to increase efficacy of the resulting therapeutic compositions or treatment. In further embodiments, the engineered sequence is useful to increase the efficacy of the functional hIDUA protein being expressed, and may also permit a lower dose of a therapeutic reagent that delivers the functional hIDUA. In certain embodiments, the engineered hIUDA coding sequence is characterized by improved translation as compared to a wild type hIDUA coding sequence.

By “engineered” is meant that the nucleic acid sequences encoding a functional hIDUA enzyme described herein are assembled and placed into any suitable genetic element, e.g., naked DNA, phage, transposon, cosmid, episome, etc., which transfers the hIDUA sequences carried thereon to a host cell, e.g., for generating non-viral delivery systems (e.g., RNA-based systems, naked DNA, or the like), or for generating viral vectors in a packaging host cell, and/or for delivery to a host cell in a subject. In certain embodiments, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of a construct, the full-length of a gene coding sequence, or a fragment of at least about 500 to 1000 nucleotides. However, identity among smaller fragments, for example, of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 100 amino acids, about 300 amino acids, or a peptide fragment thereof or the corresponding nucleic acid sequence coding sequences. A suitable amino acid fragment may be at least about 8 amino acids in length, and may be up to about 50 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Identity may be determined by preparing an alignment of sequences and through the use of a variety of algorithms and/or computer programs known in the art or commercially available (e.g., BLAST, ExPASy; Clustal Omega; FASTA; using, e.g., Needleman-Wunsch algorithm, Smith-Waterman algorithm). Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Identity or similarity with respect to a sequence is defined herein as the percentage of amino acid residues in the candidate sequence that are identical (i.e., same residue) or similar (i.e., amino acid residue from the same group based on common side-chain properties, see below) with the peptide and polypeptide regions provided herein, after aligning the sequences and introducing gaps, if necessary, to achieve the maximum percent sequence identity. Percent (%) identity is a measure of the relationship between two polynucleotides or two polypeptides, as determined by comparing their nucleotide or amino acid sequences, respectively. In general, the two sequences to be compared are aligned to give a maximum correlation between the sequences. The alignment of the two sequences is examined and the number of positions giving an exact amino acid or nucleotide correspondence between the two sequences determined, divided by the total length of the alignment and multiplied by 100 to give a % identity figure. This % identity figure may be determined over the whole length of the sequences to be compared, which is particularly suitable for sequences of the same or very similar length and which are highly homologous, or over shorter defined lengths, which is more suitable for sequences of unequal length or which have a lower level of homology. There are a number of algorithms, and computer programs based thereon, which are available to be used the literature and/or publicly or commercially available for performing alignments and percent identity. The selection of the algorithm or program is not a limitation of the present invention.

Examples of suitable alignment programs including, e.g., the software CLUSTALW under Unix and then be imported into the Bioedit program (Hall, T. A. 1999, BioEdit: a user-friendly biological sequence alignment editor and analysis program for Windows 95/98/NT. Nucl. Acids. Symp. Ser. 41:95-98); the Wisconsin Sequence Analysis Package, version 9.1 (Devereux J. et al., Nucleic Acids Res., 12:387-395, 1984, available from Genetics Computer Group, Madison, Wis., USA). The programs BESTFIT and GAP, may be used to determine the % identity between two polynucleotides and the % identity between two polypeptide sequences.

Other programs for determining identity and/or similarity between sequences include, e.g., the BLAST family of programs available from the National Center for Biotechnology Information (NCB), Bethesda, Md., USA and accessible through the home page of the NCBI at www.ncbi.nlm.nih.gov), the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used; and FASTA (Pearson W. R. and Lipman D. J., Proc. Natl. Acad. Sci. USA, 85:2444-2448, 1988, available as part of the Wisconsin Sequence Analysis Package). SeqWeb Software (a web-based interface to the GCG Wisconsin Package: Gap program).

In certain embodiments, the hIDUA coding sequence is less than 80% identical to the native hIDUA sequence of SEQ ID NO: 20, and encodes the amino acid sequence of SEQ ID NO: 21. In a further embodiment, the hIDUA coding sequence comprises a sequence that is less than 80% identical to nucleotides (nt) 88 to 1959 of SEQ ID NO: 20, and encodes amino acids 28 to 635 of SEQ ID NO: 21.

In certain embodiments the hIDUA coding sequence shares less than about 99%, less than about 98%, less than about 97%, less than about 96%, less than about 95%, less than about 94%, less than about 93%, less than about 92%, less than about 91%, less than about 90%, less than about 89%, less than about 88%, less than about 87%, less than about 86%, less than about 85%, less than about 84%, less than about 83%, less than about 82%, less than about 81%, less than about 80%, less than about 79%, less than about 78%, less than about 77%, less than about 76%, less than about 75%, less than about 74%, less than about 73%, less than about 72%, less than about 71%, less than about 70%, less than about 69%, less than about 68%, less than about 67%, less than about 66%, less than about 65%, less than about 64%, less than about 63%, less than about 62%, less than about 61% or identity with the native hIDUA coding sequence (SEQ ID NO: 20). In other embodiments, the hIDUA coding sequence shares about 99%, about 98%, about 97%, about 96%, about 95%, about 94%, about 93%, about 92%, about 91%, about 90%, about 89%, about 88%, about 87%, about 86%, about 85%, about 84%, about 83%, about 82%, about 81%, about 80%, about 79%, about 78%, about 77%, about 76%, about 75%, about 74%, about 73%, about 72%, about 71%, about 70%, about 69%, about 68%, about 67%, about 66%, about 65%, about 64%, about 63%, about 62%, about 61% or less identity with the native hIDUA coding sequence (SEQ ID NO: 20). In certain embodiments, the hIDUA coding sequence is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to SEQ ID NO: 20 and encodes a functional human alpha-L-iduronidase. In further embodiments, the hIDUA coding sequence is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% identical to SEQ ID NO: 23, 24, 25, 26, or 27 and encodes a functional human alpha-L-iduronidase.

Identity may be with respect to a sequence that encodes a full-length hIDUA (e.g., nt 1 to nt 1959 of SEQ ID NO: 20) or with respect to a sequence that encodes a mature hIDUA (e.g., nt 82 to nt 1959 of SEQ ID NO: 20). In certain embodiments, the full-length hIDUA includes the leader peptide sequences of the human alpha-L-iduronidase (i.e., encoding 1 to about amino acid 27 of SEQ ID NO: 21), corresponding to about 1 to about 81 of SEQ ID NO: 20. In another embodiment, the hIDUA gene encodes a functional synthetic human alpha-L-iduronidase enzyme which is a synthetic peptide comprising a heterologous leader sequence fused to the secreted portion of a functional alpha-L-iduronidase enzyme, i.e., about amino acid 28 to about 653 of SEQ ID NO: 21 or one of the functional variants thereof which are identified herein. In yet another embodiment, the hIDUA gene encodes a functional synthetic human alpha-L-iduronidase enzyme of SEQ ID NO: 21, wherein the leader sequence is encoded by nucleotides 1 to 81 of SEQ ID NO: 20 encoding amino acids 1 to 27 of SEQ ID NO: 21, and amino acids 28 to 653 are encoded by a sequence that is at least 85%, 95%, or 99% identical to nucleotides 82 to 1959 of SEQ ID NO: 20 or a sequence that is at least 85%, 95%, or 99% identical to nucleotides 82-1959 of SEQ ID NO: 22. In certain embodiments, the hIDUA coding sequence includes nt 1 to 1959 of SEQ ID NO: 20, or a sequence at least 85%, 90%, 95%, or 99% identical thereto which encodes a full-length hIDUA. In certain embodiments, the hIDUA coding sequence includes nt 82 to nt 1959 of SEQ ID NO: 20, or a sequence at least 85%, 90%, 95%, or 99% identical thereto encoding a function hIDUA. In certain embodiments, the hIDUA coding sequence includes nt 1 to 1959 of SEQ ID NO: 23, 24, 25, or 26, or a sequence at least 85%, 90%, 95%, or 99% identical thereto which encodes a full-length hIDUA. In certain embodiments, the hIDUA coding sequence includes nt 82 to nt 1959 of SEQ ID NO: 23, 24, 25, or 26, or a sequence at least 85%, 90%, 95%, or 99% identical thereto encoding a mature hIDUA (e.g. amino acid 27 to 653 of SEQ ID NO: 21).

In further embodiments, the hIDUA coding sequence comprises SEQ ID NO: 22, 23, 24, 25, or 26.

| | |
|---|---|
| hIDUAcoVI | atgaggcctctcagacctagagctgctctg |
| SEQ ID NO: 22 | ctggcactgctggcttctctgcttgctgct |
| | cctcctgtggctcctgccgaagctcctcat |
| | ctggtgcacgtggatgccgccagagcactg |
| | tggcccctgagaagattttggcggagcacc |
| | ggcttttgccctccactgcctcattctcag |
| | gccgaccagtacgtgctgagctgggaccag |
| | caactgaacctggcctacgtgggagccgtg |
| | cctcacagaggcattaagcaagtgcggacc |
| | cactggctgctggaactggtcacaacaaga |
| | ggcagcacaggcagaggcctgagctacaac |
| | ttcacccacctggacggctacctggacctg |
| | ctgagagagaatcagctgctgcctggcttc |
| | gagctgatgggctctgcctctggccacttc |
| | accgacttcgaggacaagcagcaggttttc |
| | gagtggaaggacctggtgtccagcctggcc |
| | agacggtacatcggcagatacggactggcc |
| | cacgtgtccaagtggaacttcgagacctgg |
| | aacgagcccgaccaccacgacttcgacaac |
| | gtgtcaatgaccatgcagggctttctgaac |
| | tactacgacgcctgcagcgagggcctgaga |
| | gctgcttctcctgctctgagacttggcggc |
| | cctggcgactcttttcacacccctccaaga |
| | agccctctgtcctggggactgctgagacac |
| | tgtcacgacggcaccaatttcttcaccggc |
| | gaggctggcgtgcggctggattatatcagc |
| | ctgcacagaaagggcgccagaagcagcatc |
| | agcatcctggaacaagagaaggtggtggcc |
| | cagcagatcagacagctgttccccaagttc |
| | gccgacacacccatctacaacgacgaggcc |
| | gatcctctcgttggctggtcacttcctcag |
| | ccttggagagccgatgtgacctatgccgcc |
| | atggtggtcaaagtgatcgcccagcaccag |
| | aatctgctgctcgccaataccaccagcgcc |
| | tttccatacgctctgctgagcaacgacaac |
| | gccttcctgagctatcaccctcatcctttc |
| | gctcagcggaccctgaccgccagattccaa |
| | gtgaacaacacccggcctccacacgtgcag |
| | ctgctgagaaaaccagtgctgacagccatg |
| | ggcctgctcgccctgctggacgaagaacaa |
| | ctgtgggccgaagtgtcccaggccggaaca |
| | gtgctggatagcaatcacacagtgggcgtg |
| | ctggcctccgctcatagacctcaaggacca |
| | gccgatgcttggagggctgccgtgctgatc |
| | tacgccagcgacgatacaagggctcacccc |
| | aacagatccgtggccgtgacactgagactg |
| | agaggcgttccaccaggacctggcctggtg |
| | tacgtgaccagatacctggacaacggcctg |
| | tgcagccctgatggcgaatggcgtagacta |
| | ggcagacctgtgtttcctaccgccgagcag |
| | ttcagacggatgagagccgctgaagatccc |
| | gtggctgctgctccaagacctcttccagct |
| | ggtggcagactgactctgaggcctgcactc |
| | agactgcctagtctgctgctggtgcacgtc |
| | tgtgccagacctgagaagcctcctggccaa |
| | gtgacaagactgagggccctgccactgaca |
| | cagggacagctggttcttgtttggagcgac |
| | gagcacgtgggcagcaagtgtctgtggacc |
| | tacgagatccagttcagccaggacggcaag |
| | gcctacacacccgtgtctagaaagcctagc |
| | accttcaacctgttcgtgttcagccccgat |
| | acaggcgccgtgtctggcagctatagagtc |
| | agagccctggactactgggccagaccagga |
| | ccattttctgaccccgtgccttacctggaa |
| | gtgcccgttcctagaggccctccttctcct |
| | ggaaatccc |
| hIDUAcoV2 | atgaggcctctcagacctagagctgctctg |
| SEQ ID NO: 23 | ctggcactgctggcttctctgcttgctgct |
| | cctcctgtggctcctgccgaagctcctcat |
| | ctggtgcacgtggatgccgccagagcactg |
| | tggcccctgagaagattttggcggagcacc |
| | ggcttttgccctccactgcctcattctcaa |
| | gccgaccaatacgtgctgagctgggaccag |
| | caactgaacctggcctacgtgggagccgtg |
| | cctcacagaggcattaagcaagtgcggacc |
| | cactggctgctggaactggtcacaacaaga |
| | ggcagcacaggcagaggcctgagctacaac |
| | ttcacccacctggacggctacctggacctg |
| | ctgagagagaatcagctgctgcctggcttc |
| | gagctgatgggctctgcctctggccacttc |
| | accgacttcgaggacaagcagcaggttttc |
| | gagtggaaggacctggtgtccagcctggcc |
| | agacgctacatcggcagatacggactggcc |
| | cacgtgtccaagtggaacttcgagacctgg |
| | aacgagcccgaccaccacgacttcgacaac |
| | gtgtcaatgaccatgcagggctttctgaac |
| | tactacgacgcctgcagcgagggcctgaga |
| | gctgcttctcctgctctgagacttggcggc |
| | cctggcgactcttttcacacccctccaaga |
| | agccctctgtcctggggactgctgagacac |
| | tgtcacgacggcaccaatttcttcaccggc |
| | gaggctggcgtgcggctggattatatcagc |
| | ctgcacagaaagggcgccagaagcagcatc |
| | agcatcctggaacaagagaaggtggtggcc |
| | cagcagatcagacagctgttccccaagttc |
| | gccgacacacccatctacaacgacgaggcc |
| | gatcctctcgttggctggtcacttcctcag |
| | ccttggagagccgatgtgacctatgccgcc |
| | atggtggtcaaagtgatcgcccagcaccag |
| | aatctgctgctcgccaataccaccagcgcc |
| | tttccatacgctctgctgagcaacgacaac |
| | gccttcctgagctatcaccctcatcctttc |
| | gctcagcggaccctgaccgccagattccaa |
| | gtgaacaacacccggcctccacacgtgcag |
| | ctgctgagaaaaccagtgctgacagccatg |
| | ggcctgctcgccctgctggacgaagaacaa |
| | ctgtgggccgaagtgtcccaggccggaaca |
| | gtgctggatagcaatcacacagtgggcgtg |
| | ctggcctccgctcatcgacctcaaggacca |
| | gccgatgcttggagggctgccgtgctgatc |
| | tacgccagcgacgatacaagggctcacccc |
| | aacagatccgtggccgtgacactgagactg |
| | agaggcgttccaccaggacctggcctggtg |
| | tacgtgaccagatacctggacaacggcctg |
| | tgcagccctgatggcgaatggcgtagacta |
| | ggcagacctgtgtttcctaccgccgagcag |
| | ttcagacggatgagagccgctgaagatccc |
| | gtggctgctgctccaagacctcttccagct |
| | ggtggcagactgactctgaggcctgcactc |
| | agactgcctagtctgctgctggtgcacgtc |
| | tgtgccagacctgagaagcctcctggccaa |
| | gtgacaagactgagggccctgccactgaca |
| | cagggacagctggttcttgtttggagcgac |
| | gagcacgtgggcagcaagtgtctgtggacc |
| | tacgagatccagttcagccaggacggcaag |
| | gcctacacacccgtgtctagaaagcctagc |
| | accttcaacctgttcgtgttcagccccgat |
| | acaggcgccgtgtctggcagctatagagtc |
| | agagccctggactactgggccagaccagga |
| | ccattttctgaccccgtgccttacctggaa |
| | gtgcccgttcctagaggccctccttctcct |
| | ggaaatccc |
| hIDUAcoV3 | atgcgacccttgcgaccaagagccgccctg |
| SEQ ID NO: 24 | cttgcgttgttggcttcccttttggctgca |
| | ccacccgtcgccccggcagaggcgcctcac |
| | ctcgtgcatgtagacgcagcccgcgccctc |
| | tggccattgcggcgattctggaggtctacc |
| | ggtttctgcccacccctgcctcattcacaa |
| | gccgaccaatacgttttgtcctgggatcaa |
| | cagctcaaccttgcgtatgtaggcgctgtt |
| | ccacaccggggaattaagcaggtccggact |
| | cactggcttcttgagttggttacgacgagg |
| | ggttcaacagggagagggttgtcctacaac |
| | tttacacatctggatggttatttagacctg |
| | ctccgagaaaaccaacttctccctggattc |
| | gagctcatgggctctgcctctggtcacttt |
| | acggatttcgaggataaacagcaagtcttc |
| | gagtggaaggatcttgtgagcagccttgcc |
| | agaagatatataggaagatacgggttggca |
| | cacgtatctaaatggaactttgagacttgg |
| | aacgaacccgaccatcacgactttgacaac |
| | gtatctatgacgatgcaaggcttcctcaac |
| | tattatgacgcgtgcagtgaaggtctgagg |
| | gctgcgtctccggcgctcagattgggaggt |
| | cctggagacagctttcatacgcctccgcga |
| | tcccctcttagctgggggttgctcagacat |
| | tgccacgacggtacaaacttcttcaccggc |
| | gaggcaggtgttaggctcgactacatctcc |
| | ctgcaccgaaagggcgcgcgatcttctatc |
| | agtatattggaacaagagaaagttgtggct |
| | caacaaattcgccaactttttcccaaattc |
| | gctgataccccgatttacaacgacgaagcc |
| | gacccattggtaggttggagtcttccccag |
| | ccttggcgagcggatgtcacctacgcagct |
| | atggtagttaaagtgattgcgcaacaccaa |
| | aatctccttcttgccaacacgacttctgcg |
| | tttccatacgcgttgttgtctaacgacaac |
| | gcctttttgtcctatcacccgcacccgttt |
| | gcgcagcgaactctgaccgcaagatttcag |
| | gttaacaatactcggccaccacacgtacag |
| | cttttgcgcaaacctgtattgactgccatg |
| | ggattgttggcactgttggatgaagaacaa |
| | ctgtgggctgaagtgagccaggcgggtaca |
| | gttctggacagtaatcacactgtaggcgtg |
| | ctcgccagtgctcaccgaccacaggggccg |
| | gcagacgcctggagagctgctgtactcatc |
| | tacgcatcagacgataccagggcgcatccc |
| | aatagatccgtcgccgtaactcttcggctt |
| | cggggcgtcccgccagggccgggccttgtt |
| | tatgttactcgatacttagacaacggactt |
| | tgtagtcctgatggtgaatggcgtagatta |
| | gggcggcctgtctttcctactgcggagcag |
| | ttcagacgaatgagggctgctgaggaccca |
| | gttgcagctgcaccccgcccgcttccggcc |
| | ggtggcagacttacgctcaggcccgcgctt |
| | aggttgccgtccttgttgcttgtccacgtt |
| | tgcgctcgcccagaaaagccgccgggacag |
| | gttacacgacttcgggctctgcccctgacg |
| | cagggacaactggtgcttgtttggagtgat |
| | gaacacgtaggaagcaagtgcttgtggact |
| | tacgagatacaattcagccaagacggcaag |
| | gcttatacccctgtttcacgcaaaccttct |
| | acttttaatttgtttgtcttttctccggat |
| | acgggggcggtctctgggtcatatcgcgtg |
| | agagcactcgattactgggctagaccaggg |
| | ccatttagcgatcccgttccttatctggag |
| | gtgcccgtcccgaggggtccaccaagcccc |
| | ggaaatccg |
| hIDUAcoV4 | atgaggcccctgaggcccagggccgccctg |
| SEQ ID NO: 25 | ctggccctgctggcctccctgctggccgct |
| | cctcccgtggccccagccgaggctcctcac |
| | ctggtgcacgtggacgccgccagggccctg |
| | tggcccctgaggaggttctggaggtccacc |
| | ggcttctgtcctcctctgccccactcccaa |
| | gccgaccaatacgtgctgtcctgggaccag |
| | cagctgaacctggcctatgtgggagctgtg |
| | ccccacaggggcatcaagcaagtgaggacc |
| | cactggctgctggagctggtgaccaccagg |
| | ggctccaccggcaggggcctgtcctacaac |
| | ttcacccacctggacggctacctggacctg |
| | ctgagggagaaccagctgctgcccggcttc |
| | gagctgatgggctccgcctccggccacttc |
| | accgacttcgaggacaagcagcaggtgttc |
| | gagtggaaggacctggtgtcctccctggcc |
| | aggagatacatcggcagatacggcctggcc |
| | cacgtgtccaagtggaacttcgagacctgg |
| | aacgagcccgaccaccacgacttcgacaac |
| | gtgtccatgaccatgcagggcttcctgaac |
| | tactacgacgcctgctccgagggcctgagg |
| | gccgcctctcctgccctgaggctgggagga |
| | cctggcgactccttccacacacctccaagg |
| | tctcccctgtcctggggcctgctgaggcac |
| | tgccacgacggcaccaacttcttcaccgga |
| | gaggctggagtgaggctggactacatctcc |
| | ctgcaccggaagggcgccaggtcctccatc |
| | tccatcctggagcaggagaaggtggtggcc |
| | cagcagatcaggcagctgttccccaagttc |
| | gccgacacacctatctacaacgacgaggcc |
| | gatcctctggtgggctggtccctgccccag |
| | ccctggagggctgatgtgacctatgctgcc |
| | atggtggtgaaggtgatcgcccagcaccag |
| | aacctgctgctggccaacaccacctccgcc |
| | ttcccctacgccctgctgtccaacgacaac |
| | gccttcctgtcctaccatcctcaccccttc |
| | gcccaaaggaccctgaccgccaggttccaa |
| | gtgaacaacaccaggcctcctcacgtgcag |
| | ctgctgaggaagcccgtgctgaccgccatg |
| | ggcctgctggccctgctggacgaggagcag |
| | ctgtgggccgaggtgtcccaggccggcacc |
| | gtgctggactccaaccacaccgtgggcgtg |
| | ctggcctccgcccaccggccccagggcccc |
| | gccgacgcctggagggccgccgtgctgatc |
| | tacgcctccgacgacaccagggcccacccc |
| | aacaggtccgtggccgtgaccctgaggctg |
| | aggggcgtgcctcccggccccggcctggtg |
| | tacgtgaccaggtatctggacaacggcctg |
| | tgctctcctgacggcgagtggaggaggctg |
| | ggcaggcccgtgttccccaccgccgagcag |
| | ttcaggaggatgagggccgccgaggacccc |
| | gtggccgccgctcctcgacccctgcctgct |
| | ggaggcaggctgaccctgaggcccgccctg |
| | aggctgccctccctgctgctggtgcacgtg |
| | tgcgccaggcccgagaagcctcctggccag |
| | gtgaccaggctgagggccctgcccctgacc |
| | cagggccagctggtgctggtgtggtccgac |
| | gagcacgtgggctccaagtgcctgtggacc |
| | tacgagatccagttctcccaggacggcaag |
| | gcctacacacctgtgtccaggaagccctcc |
| | accttcaacctgttcgtgttctctcctgac |
| | accggcgccgtgtccggctcctaccgagtg |
| | agggccctggactactgggccaggcccggc |
| | cccttctccgaccccgtgccctacctggag |
| | gtgcccgtgcccaggggacctccttctcct |
| | ggcaacccc |
| hIDUAcoV5 | atgagacctttaagaccacgtgctgctctg |
| SEQ ID NO: 26 | ctggctttactcgcttctttactggccgct |
| | cctcccgtggcccccgctgaagctcctcac |
| | ttagtgcacgtggatgccgccagagctctg |
| | tggcctctgaggagattttggaggtccact |
| | ggtttctgccctcctttaccccatagccaa |
| | gctgatcagtacgtgctgagctgggatcag |
| | caactgaatttagcctacgtcggcgctgtg |
| | cctcacagaggcattaagcaagtgaggacc |
| | cactggctgctggaactggtcaccactcgt |
| | ggcagcactggtagaggactcagctacaat |
| | ttcacacatttagacggctatctggattta |
| | ctgagagagaatcagttattacccggtttc |
| | gagctcatgggaagcgcctccggccatttc |
| | accgacttcgaggacaagcagcaagttttc |
| | gaatggaaagatttagtgtcctccctcgct |
| | cgtaggtacatcggaagatacggtttagcc |
| | cacgtcagcaagtggaacttcgagacttgg |
| | aatgagcccgatcaccacgattttgacaat |
| | gtcagcatgaccatgcaaggttttttaaac |
| | tactacgatgcttgtagcgaaggcctcaga |
| | gctgccagccccgctctgagactcggcgga |
| | cccggtgactccttccacacacctcctaga |
| | agccctttaagctggggtttactgagacac |
| | tgtcacgacggcaccaacttctttaccggc |
| | gaggccggcgttcgtctcgactatatctct |
| | ttacatcgtaagggcgctcgttcctccatt |
| | tccatcctcgaacaagaaaaggtggtggct |
| | cagcagattaggcaactcttccccaagttc |
| | gccgacacccctatctataatgacgaggct |
| | gatcctctggtcggctggtctttaccccaa |
| | ccttggagagctgatgtcacctacgctgcc |
| | atggtggtgaaggtgatcgcccagcaccag |
| | aatttattattagctaacaccacatccgcc |
| | ttcccttacgctctgctgtccaacgataac |
| | gccttcctcagctatcaccctcaccccttt |
| | gcccagagaactttaaccgctagattccaa |
| | gttaataacaccagacccccccacgtccag |
| | ttattacgtaagcccgttctgacagccatg |
| | ggtttactcgctttactggacgaagagcag |
| | ctgtgggctgaagtgagccaagctggcacc |
| | gtgctggatagcaaccacacagtgggcgtg |
| | ctcgccagcgctcataggcctcaaggaccc |
| | gctgatgcttggagggctgccgtgctgatc |
| | tacgccagcgacgacacaagggctcacccc |
| | aataggtccgtggctgttacactgagactc |
| | agaggcgtcccccccggtcccggtttagtg |
| | tatgtgaccagatatttagacaacggactg |
| | tgctcccccgacggagagtggagaagactg |
| | ggtcgtcccgtgtttcctaccgccgagcag |
| | tttaggaggatgagagctgccgaggatccc |
| | gttgccgccgccccacgtcctttacccgcc |
| | ggcggaaggctgacattaagacccgcttta |
| | agactgccctctttactgctcgtgcatgtg |
| | tgtgccagacccgaaaagcctcccggacaa |
| | gttaccagactgagagccctccctctgacc |
| | caaggtcagctggtgctggtgtggtccgac |
| | gaacacgtgggcagcaagtgtttatggacc |
| | tatgagatccagttctcccaagatggcaaa |
| | gcttacacccccgtgtctcgtaaacctagc |
| | accttcaatttattcgtgtttagccccgac |
| | accggagccgtgtccggcagctatcgtgtg |
| | agagctttagactactgggctaggcccggc |
| | ccttttagcgatcccgtgccttatttagaa |
| | gtgcccgttcccagaggaccccccagcccc |
| | ggaaatcct |

As used herein, “a desired function” refers to an hIDUA enzyme activity at least about 20%, about 25%, about 30%, about 35%, about 40%, about 45%, about 50%, about 55%, about 60%, about 65%, about 70%, about 75%, about 80%, about 85%, about 90%, about 95%, or about 100% of a healthy control.

As used herein, the phrases “ameliorate a symptom” and “improve a symptom”, and grammatical variants thereof, refer to reversal of a MPSI, Hurler, and/or Hurler-Scheie and/or Scheie syndome-related symptom, slowdown or prevention of progression of a MPSI, Hurler, and/or Hurler-Scheie and/or Scheie syndome-related symptom. In certain embodiments, the amelioration or improvement refers to the total number of symptoms in a patient after administration of the described composition(s) or use of the described method, which is reduced by about 5%, about 10%, about 20%, about 30%, about 40%, about 50%, about 60%, about 70%, about 80%, about 90%, about 95% compared to that before the administration or use. In another embodiment, the amelioration or improvement refers to the severity or progression of a symptom after administration of the described composition(s) or use of the described method, which is reduced by about 5%, about 10%, about 20%, about 30%, about 40%, about 50%, about 60%, about 70%, about 80%, about 90%, about 95% compared to that before the administration or use.

It should be understood that the compositions in the functional hIDUA or an hIDUA coding sequence described herein are intended to be applied to other compositions, regiments, aspects, embodiments, and methods described across the Specification.

### Expression Cassettes

In certain embodiments, provided herein are expression cassettes having a nucleic acid sequence encoding a functional hIDUA and a regulatory sequence which directs the expression thereof. As used herein, an “expression cassette” refers to a nucleic acid molecule which comprises a sequence encoding a hIDUA gene, promoter, and may include other regulatory sequences therefor, which cassette may be delivered via a genetic element (e.g., a plasmid) to a packaging host cell and packaged into the capsid of a viral vector (e.g., a viral particle). Typically, such an expression cassette for generating a viral vector contains the hIDUA coding sequence described herein flanked by packaging signals of the viral genome and other expression control sequences such as those described herein. In certain embodiments, an expression cassette is provided that includes a nucleic acid sequence encoding a functional gene product (e.g., hIDUA) operably linked to regulatory sequences which direct its expression in a target cell and miRNA target sequences in the 3′ and/or 5′ UTR. As described herein, the miRNA target sequences are designed to be specifically recognized by miRNA present in cells in which transgene expression is undesirable and/or reduced levels of transgene expression are desired. In certain embodiments, the miRNA target sequences specifically reduce expression of the transgene in dorsal root ganglion. In certain embodiments, the miRNA target sequences are located in the 3′ UTR, 5′ UTR, and/or in both 3′ and 5′ UTR.

As used herein, the term “expression” or “gene expression” refers to the process by which information from a gene is used in the synthesis of a functional gene product. The gene product may be a protein, a peptide, or a nucleic acid polymer (such as a RNA, a DNA or a PNA).

As used herein, the term “regulatory sequence”, or “expression control sequence” refers to nucleic acid sequences, such as initiator sequences, enhancer sequences, and promoter sequences, which induce, repress, or otherwise control the transcription of protein encoding nucleic acid sequences to which they are operably linked.

As used herein, the term “operably linked” refers to both expression control sequences that are contiguous with the nucleic acid sequence encoding a gene product and/or expression control sequences that act in trans or at a distance to control the transcription and expression thereof.

A “5′ UTR” is upstream of the initiation codon for a gene product coding sequence. The 5′ UTR is generally shorter than the 3′ UTR. Generally, the 5′ UTR is about 3 nucleotides to about 200 nucleotides in length, but may optionally be longer.

A “3′ UTR” is downstream of the coding sequence for a gene product and is generally longer than the 5′ UTR. In certain embodiments, the 3′ UTR is about 200 nucleotides to about 800 nucleotides in length, but may optionally be longer or shorter.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g. a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, vector genome, expression cassette, or vector, indicates that the protein or the nucleic acid is present with another sequence or subsequence which with which the protein or nucleic acid in question is not found in the same relationship to each other in nature.

In one embodiment, the expression cassette provided is designed for expression and secretion in the central nervous system (CNS), including the cerebral spinal fluid and brain. In a particularly desired embodiment, the expression cassette is useful for expression in both the CNS and in the liver, thereby allowing treatment of both the systemic and CNS-related effects of MPSI, Hurler, Hurler-Scheie and Scheie syndromes. For example, the inventors have observed that certain constitutive promoters (e.g., CMV) do not drive expression at desired levels when delivered intrathecally, thereby providing suboptimal hIDUA expression levels. However, the chicken beta-actin promoter drives expression well both upon intrathecal delivery and systemic delivery. Thus, this is a particularly desirable promoter. Other promoters may be selected, but expression cassettes containing same may not have all of the advantages of those with a chicken beta-actin promoter. A variety of chicken beta-actin promoters have been described alone, or in combination with various enhancer elements (e.g., CB7 is a chicken beta-actin promoter with cytomegalovirus enhancer elements, a CAG promoter, which includes the promoter, the first exon and first intron of chicken beta actin, and the splice acceptor of the rabbit beta-globin gene), a CBh promoter [S J Gray et al, Hu Gene Ther, 2011 September; 22(9): 1143-1153]. In other embodiments, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kugler S et al, Human synapsin 1 gene promoter confers highly neuron-specific long-term transgene expression from an adenoviral vector in the adult rat brain depending on the transduced area. Gene Ther. 2003 February; 10(4):337-47), a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol. 2016 January; 58(1):30-6. doi: 10.1007/s12033-015-9899-5).

Examples of promoters that are tissue-specific are well known for liver and other tissues (albumin, Miyatake et al., (1997) J. Virol., 71:5124-32; hepatitis B virus core promoter, Sandig et al., (1996) Gene Ther., 3:1002-9; alpha-fetoprotein (AFP), Arbuthnot et al., (1996) Hum. Gene Ther., 7:1503-14), bone osteocalcin (Stein et al., (1997) Mol. Biol. Rep., 24:185-96); bone sialoprotein (Chen et al., (1996) J. Bone Miner. Res., 11:654-64), lymphocytes (CD2, Hansal et al., (1998) J. Immunol., 161:1063-8; immunoglobulin heavy chain; T cell receptor chain), neuronal such as neuron-specific enolase (NSE) promoter (Andersen et al., (1993) Cell. Mol. Neurobiol., 13:503-15), neurofilament light-chain gene (Piccioli et al., (1991) Proc. Natl. Acad. Sci. USA, 88:5611-5), and the neuron-specific vgf gene (Piccioli et al., (1995) Neuron, 15:373-84), among others. Alternatively, a regulatable promoter may be selected. See, e.g., WO 2011/126808B2, incorporated by reference herein.

In one embodiment, the expression cassette ocomprises one or more expression enhancers. In one embodiment, the expression cassette contains two or more expression enhancers. These enhancers may be the same or may be different. For example, an enhancer may include an Alpha mic/bik enhancer or a CMV enhancer. This enhancer may be present in two copies which are located adjacent to one another. Alternatively, the dual copies of the enhancer may be separated by one or more sequences. In still another embodiment, the expression cassette further contains an intron, e.g., a chicken beta-actin intron, a human β-globulin intron, and/or a commercially available Promega® intron. Other suitable introns include those known in the art, e.g., such as are described in WO 2011/126808.

Further, an expression cassette provided includes a suitable polyadenylation signal. In one embodiment, the polyA sequence is a rabbit globulin poly A. See, e.g., WO 2014/151341. Alternatively, another polyA, e.g., a human growth hormone (hGH) polyadenylation sequence, an SV50 polyA, or a synthetic polyA. Still other conventional regulatory elements may be additional or optionally included in an expression cassette or vector genome.

In one embodiment, the regulatory sequence further comprises an enhancer. In one embodiment, the regulatory sequence comprises one enhancer. In another embodiment, the regulatory sequence contains two or more expression enhancers. These enhancers may be the same or may be different. For example, an enhancer may include an Alpha mic/bik enhancer or a CMV enhancer. This enhancer may be present in two copies which are located adjacent to one another. Alternatively, the dual copies of the enhancer may be separated by one or more sequences.

In one embodiment, the regulatory sequence further comprises an intron. In a further embodiment, the intron is a chicken beta-actin intron. Other suitable introns include those known in the art may by a human β-globulin intron, and/or a commercially available Promega® intron, and those described in WO 2011/126808.

In one embodiment, the regulatory sequence further comprises a Polyadenylation signal (polyA). In a further embodiment, the polyA is a rabbit globin poly A. See, e.g., WO 2014/151341. Alternatively, another polyA, e.g., a human growth hormone (hGH) polyadenylation sequence, an SV40 polyA, or a synthetic polyA may be included in an expression cassette.

In certain embodiments, the expression cassette is designed for expression in a human subject while reducing or eliminating DRG-expression of the transgene product.

In one embodiment, the expression cassette is designed for expression in the central nervous system (CNS), including the cerebral spinal fluid and brain. In certain embodiments, the expression cassette is designed for expression or enhanced expression of the transgene in one or more cell type present in the CNS (excluding the dorsal root ganglia), including nerve cells (such as, pyramidal, purkinje, granule, spindle, and interneuron cells) and glia cells (such as astrocytes, oligodendrocytes, microglia, and ependymal cells). In certain embodiments, enhanced expression of the transgene is achieved in one or more cell type with little to no expression of the transgene in another cell type of the CNS. In certain embodiments, the expression cassette is useful for expression in cells other than those of the CNS.

As used herein, an “miRNA” refers to a microRNA which is a small non-coding RNA molecule which regulates mRNA and stops it from being translated to protein. The miRNA contains a “seed sequence” which is a region of nucleotides which specifically binds to mRNA by complementary base pairing, leading to destruction or silencing of the mRNA. In certain embodiments, the seed sequence is located on the mature miRNA (5′ to 3′) and is generally located at position 2 to 7 or 2 to 8 (from the 5′ end of the sense (+) strand) of the miRNA, although it may be longer than in length. In certain embodiments, the length of the seed sequence is no less than about 30% of the length of the miRNA sequence, which may be at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about 20 to about 26 nucleotides, about 22 nucleotides, about 24 nucleotides, or about 26 nucleotides.

As used herein, an “miRNA target sequence” is a sequence located on the DNA positive strand (5′ to 3′) and is at least partially complementary to a miRNA sequence, including the miRNA seed sequence. The miRNA target sequence is exogenous to the untranslated region of the encoded transgene product and is designed to be specifically targeted by miRNA in cells in which repression of transgene expression is desired. The term “miR183 cluster target sequence” refers to a target sequence that responds to one or members of the miR183 cluster (alternatively termed family), including miRs-183, -96 and -182 (as described by Dambal, S. et al. Nucleic Acids Res 43:7173-7188, 2015, which is incorporated herein by reference).Without wishing to be bound by theory, the messenger RNA (mRNA) for the transgene (encoding the gene product) is present in a cell type to which the expression cassette containing the miRNA is delivered, such that specific binding of the miRNA to the 3′ UTR miRNA target sequences results in mRNA silencing and cleavage, thereby reducing or eliminating transgene expression only in the cells that express the miRNA.

Typically, the miRNA target sequence is at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about 20 to about 26 nucleotides, about 22 nucleotides, about 24 nucleotides, or about 26 nucleotides, and which contains at least one consecutive region (e.g., 7 or 8 nucleotides) which is complementary to the miRNA seed sequence. In certain embodiments, the target sequence comprises a sequence with exact complementarity (100%) or partial complementarity to the miRNA seed sequence with some mismatches. In certain embodiments, the target sequence comprises at least 7 to 8 nucleotides which are 100% complementary to the miRNA seed sequence. In certain embodiments, the target sequence consists of a sequence which is 100% complementary to the miRNA seed sequence. In certain embodiments, the target sequence contains multiple copies (e.g., two or three copies) of the sequence which is 100% complementary to the seed sequence. In certain embodiments, the region of 100% complementarity comprises at least 30% of the length of the target sequence. In certain embodiments, the remainder of the target sequence has at least about 80% to about 99% complementarity to the miRNA. In certain embodiments, in an expression cassette containing a DNA positive strand, the miRNA target sequence is the reverse complement of the miRNA. In certain embodiments, provided herein are engineered expression cassettes o comprising at least one copy of an miR target sequence directed to one or more members of the miR-183 family or cluster operably linked to a transgene to repress expression of the transgene in DRG and/or reduce or eliminate DRG toxicity and/or axonopathy. In certain embodiments, the engineered expression cassette comprises multiple miRNA target sequences, such that the number of miRNA target sequences is sufficient to reduce or minimize transgene expression in DRG to reduce and/or eliminate DRG toxicity and/or axonopathy. The expression cassette may be delivered via any suitable carrier system, viral vector or non-viral vector, via any route, but is particularly useful for intrathecal administration.

Unexpectedly, compositions comprising the miR-183 target sequences described herein for repressing expression in the DRG have been observed to provide enhanced transgene expression in one or more different cell types (other than the DRG) within the central nervous system, including, but not limited to, neurons (including, e.g., pyramidal, purkinje, granule, spindle, and interneuron cells) or glial cells (including, e.g., astrocytes, oligodendrocytes, microglia, and ependymal cells). While this observation was initially made following an intrathecal delivery route, this expression-enhancing effect is not limited to CNS-delivery routes. Enhanced expression has also been observed following intravenous delivery and may also be achieved using other routes, e.g., intravenous (e.g., particularly high dose delivery), intramuscular (particularly high dose delivery), or other systemic delivery routes. In certain embodiments, compositions comprising the miR-183 target sequences described herein provide enhanced transgene expression in heart tissue. For example, in the studies described below, a statistically significant reduction of transgene expression is observed in dorsal route ganglia with a mir183-target containing vector compared with the control vector. Unexpectedly, expression was enhanced in the lumbar motor neurons and cerebellum. In certain embodiments, a reduction of pathology across DRG and/or eight other regions may be achieved, dorsal spinal axonopathy at cervical, thoracic, and lumbar spine, and axonopathy of median, peroneal, and radial nerves.

In certain embodiments, one may wish to select miR-182 target sequences and/or miR-96 target sequences for expression cassettes comprising transgenes which are not targeted to the CNS, so as to avoid enhancing CNS expression of the transgene (while repressing DRG expression). For example, expression cassettes comprising transgenes for delivery to skeletal muscle or the liver may wish to avoid any enhancement of CNS expression, but prevent DRG-toxicity and/or axonopathy which can be associated with the high doses which may be required.

In certain embodiments, the expression cassette contains at least one miRNA target sequence that is a miR-183 target sequence. In certain embodiments, the expression cassette contains an miR-183 target sequence that includes AGTGAATTCTACCAGTGCCATA (SEQ ID NO:1), where the sequence complementary to the miR-183 seed sequence is underlined. In certain embodiments, the expression cassette contains more than one copy (e.g. two or three copies) of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 1 and, thus, when aligned to SEQ ID NO: 1, there are one or more mismatches. In certain embodiments, a miR-183 target sequence comprises a sequence having at least 1, 2, 3, 4, 5, 6, 7, 8, 9, or 10 mismatches when aligned to SEQ ID NO: 1, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette includes a miR-183 target sequence that comprises a truncated SEQ ID NO: 1, i.e., a sequence that lacks at least 1, 2, 3, 4, 5, 6, 7, 8, 9, or 10 nucleotides at either or both the 5′ or 3′ ends of SEQ ID NO: 1. In certain embodiments, the expression cassette comprises a transgene and one miR-183 target sequence. In yet other embodiments, the expression cassette comprises at least two, at least three, at least four, at least five, at least six, or at least seven, or at least eight miR-183 target sequences. In certain embodiments, the expression cassette comprises eight miR-183 target sequences.

In certain embodiments, the expression cassette includes a combination of miRNA target sequences. In certain embodiments, the combination of target sequences includes different target sequences with at least partial complementarity for the same miRNA (such as miR-183). In certain embodiments, the expression cassette includes a combination of miRNA target sequences selected from miR-183, miR-182, and/or miR-96 target sequences as provided herein. In certain embodiments, the expression cassette comprises a transgene and two, three, or four miR-96 target sequences. In certain embodiments, an expression cassette comprises a transgene and two, three, four, five, six, seven, or eight miR-182 target sequences. In certain embodiments, the expression cassette comprises eight miR-182 target sequences. In certain embodiments, an expression cassette comprises at least one, at least two, at least three, or at least four miR-183 target sequences, optionally in combination with at least one, at least two, at least three, or at least four miR-182 target sequences, and/or optionally in combination with at least one, at least two, at least three, or at least four miR-96 target sequences. Compositions comprising a transgene and an miR-182 have been observed to minimize or eliminate dorsal root ganglia toxicity and/or prevent axonopathy. However, while effective for this purpose, the expression cassettes containing miR-182 target sequence have not been observed to enhance CNS expression as was unexpectedly found in the composited which had the miR-183 target sequence. Thus, these compositions may be desirable for genes to be targeted outside the CNS.

In certain embodiments, provided herein is an expression cassette comprising one or more miR-183 family target sequences and lacks a transgene (i.e. the miR-183 family target sequence(s) is not operably linked to a sequence encoding a heterologous gene product).

In certain embodiments, the expression cassette contains at least one miRNA target sequence that is a miR-182 target sequence. In certain embodiments, the expression cassette contains an miR-182 target sequence that includes AGTGTGAGTTCTACCATTGCCAAA (SEQ ID NO: 3). In certain embodiments, the expression cassette contains more than one copy (e.g. two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 3 and, thus, when aligned to SEQ ID NO: 3, there are one or more mismatches. In certain embodiments, a miR-183 target sequence comprises a sequence having at least 1, 2, 3, 4, 5, 6, 7, 8, 9, or 10 mismatches when aligned to SEQ ID NO: 3, where the mismatches may be non-contiguous. In certain embodiments, a miR-182 target sequence includes a region of 100% complementarity which also comprises at least 30% of the length of the miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette includes a miR-182 target sequence that comprises a truncated SEQ ID NO: 3, i.e., a sequence that lacks at least 1, 2, 3, 4, 5, 6, 7, 8, 9, or 10 nucleotides at either or both the 5′ or 3′ ends of SEQ ID NO: 3. In certain embodiments, the expression cassette comprises a transgene and one miR-182 target sequence. In yet other embodiments, the expression cassette comprises at least two, three or four miR-182 target sequences.

In certain embodiments, an expression cassette has two or more consecutive miRNA target sequences are continuous and not separated by a spacer. In certain embodiments, wherein two or more of the miRNA target sequences are separated by a spacer. In certain embodiments, the spacer is a non-coding sequence of about 1 to about 12 nucleotides, or about 2 to about 10 nucleotides in length, or about 3 to about 10 nucleotides, about 4 to about 6 nucleotide in length, or 3, 4, 5, 6, 7, 8, 9, 10 or 11 nucleotide in length.. Optionally, a single expression cassette may contain three or more miRNA target sequences, optionally having different spacer sequences therebetween. In certain embodiments, one or more spacer is independently selected from (i) GGAT (SEQ ID NO:5); (ii) CACGTG (SEQ ID NO: 6); or (iii) GCATGC (SEQ ID NO: 7). In certain embodiments, a spacer is located 3′ to the first miRNA target sequence and/or 5′ to the last miRNA target sequence. In certain embodiments, the spacers between the miRNA target sequences are the same.

In certain embodiments, an expression cassette comprises a transgene and one miR-183 target sequence and one or more different miRNA target sequences. In certain embodiments, expression cassettes contains miR-96 target sequence: mRNA and on DNA positive strand (5′ to 3′): AGCAAAAATGTGCTAGTGCCAAA (SEQ ID NO: 2); miR-182 target sequence: mRNA and on DNA positive strand (5′ to 3′): and/or AGTGTGAGTTCTACCATTGCCAAA (SEQ ID NO: 3).

Although miR-145 has been associated with brain in the literature, the studies to date have shown that miR-145 target sequences have no effect in reducing transgene expression in dorsal root ganglia. miR-145 target sequence: mRNA and on DNA positive strand (5′ to 3′): AGGGATTCCTGGGAAAACTGGAC (SEQ ID NO: 4).

As provided herein, expression cassettes contain transgenes operably linked, or under the control, of regulatory sequences which direct expression of the transgene product in the target cell. In certain embodiments, the expression cassette contains a transgene that is operably linked to one or more miRNA target sequences provided herein. In certain embodiments, the expression cassette or is designed to contain multiple miRNA target sequences. The miRNA target sequences are incorporated into the UTR of the transgene (i.e., 3′ or downstream of the gene open reading frame).

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence.

As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC.

In certain embodiments, the tandem repeats contain two, three, four, five, six, seven, eight, or more of the same miRNA target sequence. In certain embodiments, the tandem repeats have up to eight miRNA target sequences which may be the same for different. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different.

In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ may contain tandem repeats and the 5′ UTR may contain at least one miRNA target sequence.

In certain embodiments, the expression cassette contains two, three, four or more tandem repeats which start within about 0 to 20 nucleotides of the stop codon for the transgene. In other embodiments, the expression cassette contains the miRNA tandem repeats at least 100 to about 4000 nucleotides from the stop codon for the transgene.

As used herein, a “vector genome” refers to the nucleic acid sequence packaged inside a viral vector. In one example, a “vector genome” contains, at a minimum, from 5′ to 3′, a vector-specific sequence, a nucleic acid sequence encoding a functional gene product operably linked to regulatory control sequences which direct it expression in a target cell and miRNA target sequences in the untranslated region(s) and a vector-specific sequence. For example, an AAV vector genome contain inverted terminal repeat sequences and an expression cassette which comprises, e.g., a nucleic acid sequence encoding a functional gene product operably linked to regulatory control sequences which direct it expression in a target cell and miRNA target sequences in the untranslated region(s). As described herein, the miRNA target sequences are designed to be specifically recognized by miRNA sequences in cells in which transgene expression is undesirable (e.g., dorsal root ganglia) and/or reduced levels of transgene expression are desired.

In certain embodiments, provided is an rAAV with a vector genome containing a hIDUA sequence as provided herein. In further embodiments, the vectors genome comprises SEQ ID NO: 14 or SEQ ID NO: 16. In each, the vector genome includes 5′ and 3′ ITRs. Further, each contains a promoter, enhancer, hIDUA gene, and a polyA.

It should be understood that the expression cassettes described herein are intended to be applied to other compositions, regiments, aspects, embodiments and methods described across the Specification.

### Vectors

In one aspect, provided herein is a vector comprising a nucleic acid sequence encoding a functional hIUDA. In certain embodiments, the vector comprises an expression cassette as described herein for delivery of a hIDUA coding sequence.

A “vector” as used herein is a biological or chemical moiety comprising a nucleic acid sequence which can be introduced into an appropriate target cell for replication or expression of said nucleic acid sequence. Examples of a vector include but not limited to a recombinant virus, a plasmid, lipoplexes, a polymersome, polyplexes, a dendrimer, a cell penetrating peptide (CPP) conjugate, a magnetic particle, or a nanoparticle. In certain embodiments, a vector is a nucleic acid molecule into which an engineered nucleic acid encoding a functional hIDUA may be inserted, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

In certain embodiments, the vector is a non-viral plasmid that comprises an expression cassette described herein (for example, “naked DNA”, “naked plasmid DNA”, RNA, and mRNA, which may be coupled with various compositions and nano particles, including, for examples, micelles, liposomes, cationic lipid—nucleic acid compositions, poly-glycan compositions and other polymers, lipid and/or cholesterol-based-nucleic acid conjugates) and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiments, the vector described herein is a “replication-defective virus” or a “viral vector” which refers to a synthetic or artificial viral particle in which an expression cassette containing a nucleic acid sequence encoding hIDUA is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the nucleic acid sequence encoding hIDUA flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant viral vector is any suitable viral vector. The examples provide illustrative recombinant adeno-associated viruses (rAAV). Other suitable viral vectors may include, e.g., an adenovirus, a poxvirus, a bocavirus, a hybrid AAV/bocavirus, a herpes simplex virus, or a lentivirus. In preferred embodiments, these recombinant viruses are replication incompetent.

Expression cassettes can be delivered via any suitable non-viral vector delivery system or by a suitable viral vector. Suitable non-viral vector delivery systems are known in the art (see, e.g., Ramamoorth and Narvekar. J Clin Diagn Res. 2015 January; 9(1):GE01-GE06, which is incorporated herein by reference) and can be readily selected by one of skill in the art and may include, e.g., naked DNA, naked RNA, dendrimers, PLGA, polymethacrylate, an inorganic particle, a lipid particle, a polymer-based vector, or a chitosan-based formulation.

In certain embodiments, a host cell containing a nucleic acid encoding an hIDUA sequence is provided. In certain embodiments, the host cell contains a plasmid having an hIDUA-coding sequence as described herein.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

In certain embodiments, a host cell contains an expression cassette for production of hIDUA such that the protein is produced in sufficient quantities in vitro for isolation or purification. In certain embodiments, the host cell contains an expression cassette encoding hIDUA (including, for example, a functional fragment thereof). As provided herein, hIDUA polypeptide may be included in a pharmaceutical composition administered to a subject as a therapeutic (i.e., enzyme replacement therapy).

As used herein, the term “target cell” refers to any cell in which expression of the functional hIDUA is desired. In certain embodiments, the term “target cell” is intended to reference the cells of the subject being treated for MPSI, Hurler, Hurler-Scheie and/or Scheie syndrome. Examples of target cells may include, but are not limited to, liver cells, kidney cells, smooth muscle cells, and neurons. In certain embodiments, the vector is delivered to a target cell ex vivo. In certain embodiments, the vector is delivered to the target cell in vivo.

It should be understood that the compositions in the vector described herein are intended to be applied to other compositions, regiments, aspects, embodiments, and methods described across the Specification.

### Recombinant Adeno-Associated Viral (AAV) Vectors

In one aspect, provided herein is a recombinant AAV (rAAV) comprising an AAV capsid and a vector genome packaged therein.

In one embodiment, the regulatory sequence is as described above. In one embodiment, the vector genome comprises an AAV 5′ inverted terminal repeat (ITR), an expression cassette as described herein, and an AAV 3′ ITR. In one embodiment, the vector genome refers to the nucleic acid sequence packaged inside a rAAV capsid forming an rAAV vector. Such a nucleic acid sequence contains AAV inverted terminal repeat sequences (ITRs) flanking an expression cassette. In one example, a “vector genome” contains, at a minimum, from 5′ to 3′, an AAV 5′ ITR, a nucleic acid sequence encoding a functional gene product operably linked to regulatory control sequences which direct it expression in a target cell and miRNA target sequences in the untranslated region(s) and an AAV 3′ ITR. In certain embodiments, the ITRs are from AAV2 and the capsid is from a different AAV. Alternatively, other ITRs may be used. As described herein, the miRNA target sequences are designed to be specifically recognized by miRNA sequences in cells in which transgene expression is undesirable and/or reduced levels of transgene expression are desired.

The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (AITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the NAGLU coding sequences and any regulatory sequences, and an AAV 3′ ITR. However, other configurations of these elements may be suitable. A shortened version of the 5′ ITR, termed AITR, has been described in which the D-sequence and terminal resolution site (trs) are deleted. In other embodiments, the full-length AAV 5′ and 3′ ITRs are used.

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV DNase-resistant particle having an AAV protein capsid into which is packaged expression cassette flanked by AAV inverted terminal repeat sequences (ITRs) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. See, e.g., US Patent Application Publication No. 2007/0036760 A1; US Patent Application Publication No. 2009/0197338 A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689 and WO 2003/042397 (rh.10). These documents also describe other AAV which may be selected for generating AAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models. Unless otherwise specified, the AAV capsid, ITRs, and other selected AAV components described herein, may be readily selected from among any AAV, including, without limitation, the AAVs commonly identified as AAV1, AAV2, AAV3, AAV4, AAV5, AAV6, AAV6.2, AAV7, AAV8, AAV9, AAV8 bp, AAVrh10, AAVhu37, AAV7M8 and AAVAnc80, AAVrh90 (PCT/US20/30273, filed Apr. 28, 2020), AAVrh91 (PCT/US20/30266, filed Apr. 28, 2020), and AAVrh92, rh93, and rh91.93 (PCT/US20/30281, filed Apr. 28, 2020), and variants of any of the known or mentioned AAVs or AAVs yet to be discovered or variants or mixtures thereof. See, e.g., WO 2005/033321, which is incorporated herein by reference. In one embodiment, the AAV capsid is an AAV9 capsid or variant thereof. In certain embodiments, the capsid protein is designated by a number or a combination of numbers and letters following the term “AAV” in the name of the rAAV vector.

As used herein, relating to AAV, the term “variant” means any AAV sequence which is derived from a known AAV sequence, including those with a conservative amino acid replacement, and those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity with an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, Va.). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

As used herein, the terms “rAAV” and “artificial AAV” used interchangeably, mean, without limitation, an AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

As used herein, “AAV9 capsid” refers to the AAV9 having the amino acid sequence of (a) GenBank accession: AAS99264, is incorporated by reference herein and the AAV vp1 capsid protein is reproduced in SEQ ID NO: 19, and/or (b) the amino acid sequence encoded by the nucleotide sequence of GenBank Accession: AY530579.1: (nt 1 . . . 2211) (reproduced in SEQ ID NO: 18). Some variation from this encoded sequence is encompassed by the present invention, which may include sequences having about 99% identity to the referenced amino acid sequence in GenBank accession: AAS99264 and U.S. Pat. No. 7,906,111 (also WO 2005/033321) (i.e., less than about 1% variation from the referenced sequence). Such AAV may include, e.g., natural isolates (e.g., hu68, hu31 or hu32), or variants of AAV9 having amino acid substitutions, deletions or additions, e.g., including but not limited to amino acid substitutions selected from alternate residues “recruited” from the corresponding position in any other AAV capsid aligned with the AAV9 capsid; e.g., such as described in U.S. Pat. Nos. 9,102,949, 8,927,514, US 2015/349911; WO 2016/049230A11; U.S. Pat. Nos. 9,623,120; 9,585,971. However, in other embodiments, other variants of AAV9, or AAV9 capsids having at least about 95% identity to the above-referenced sequences may be selected. See, e.g., US Published Patent Application No. 2015/0079038. Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

AAVhu68 varies from another Clade F virus AAV9 by two encoded amino acids at positions 67 and 157 of vp1, SEQ ID NO: 9. In contrast, the other Clade F AAV (AAV9, hu31, hu31) have an Ala at position 67 and an Ala at position 157. Provided are novel AAVhu68 capsids and/or engineered AAV capsids having valine (Val or V) at position 157 based on the numbering of SEQ ID NO: 9 and optionally, a glutamic acid (Glu or E) at position 67. See, also, WO 2018/160582, which is incorporate by reference herein in its entirety (which includes the sequence listing).

As used herein, the term “lade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another Glade, or is outside these clades. See, e.g., G Gao, et al, J Virol, 2004 June; 78 (10: 6381-6388, which identifies Clades A, B, C, D, E and F, and provides nucleic acid sequences of novel AAV, GenBank Accession Numbers AY530553 to AY530629. See, also, WO 2005/033321.

In certain embodiments, an AAVhu68 capsid is further characterized by one or more of the following. AAVhu68 capsid proteins comprise: AAVhu68 vp1 proteins produced by expression from a nucleic acid sequence which encodes the predicted amino acid sequence of 1 to 736 of SEQ ID NO: 9, vp1 proteins produced from SEQ ID NO: 9, or vp1 proteins produced from a nucleic acid sequence at least 70% identical to SEQ ID NO: 8 which encodes the predicted amino acid sequence of 1 to 736 of SEQ ID NO: 9; AAVhu68 vp2 proteins produced by expression from a nucleic acid sequence which encodes the predicted amino acid sequence of at least about amino acids 138 to 736 of SEQ ID NO: 9, vp2 proteins produced from a sequence comprising at least nucleotides 412 to 2211 of SEQ ID NO: 8, or vp2 proteins produced from a nucleic acid sequence at least 70% identical to at least nucleotides 412 to 2211 of SEQ ID NO: 8 which encodes the predicted amino acid sequence of at least about amino acids 138 to 736 of SEQ ID NO: 9, and/or AAVhu68 vp3 proteins produced by expression from a nucleic acid sequence which encodes the predicted amino acid sequence of at least about amino acids 203 to 736 of SEQ ID NO: 9, vp3 proteins produced from a sequence comprising at least nucleotides 607 to 2211 of SEQ ID NO: 8, or vp3 proteins produced from a nucleic acid sequence at least 70% identical to at least nucleotides 607 to 2211 of SEQ ID NO: 8 which encodes the predicted amino acid sequence of at least about amino acids 203 to 736 of SEQ ID NO: 9.

The AAVhu68 vp1, vp2 and vp3 proteins are typically expressed as alternative splice variants encoded by the same nucleic acid sequence which encodes the full-length vp1 amino acid sequence of SEQ ID NO: 9 (amino acid 1 to 736). Optionally the vp1-encoding sequence is used alone to express the vp1, vp2 and vp3 proteins. Alternatively, this sequence may be co-expressed with one or more of a nucleic acid sequence which encodes the AAVhu68 vp3 amino acid sequence of SEQ ID NO: 9 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 8), or a sequence at least 70% to at least 99% (e.g., at least 85%, at least 90%, at least 95%, at least 97%, at least 98% or at least 99%) identical to SEQ ID NO: 8 which encodes aa 203 to 736 of SEQ ID NO: 9. Additionally, or alternatively, the vp1-encoding and/or the vp2-encoding sequence may be co-expressed with the nucleic acid sequence which encodes the AAVhu68 vp2 amino acid sequence of SEQ ID NO: 9 (about aa 138 to 736) without the vp1-unique region (about aa 1 to about 137), or a strand complementary thereto, the corresponding mRNA or tRNA (nt 412 to 22121 of SEQ ID NO: 8), or a sequence at least 70% to at least 99% (e.g., at least 85%, at least 90%, at least 95%, at least 97%, at least 98% or at least 99%) identical to SEQ ID NO: 8 which encodes about aa 138 to 736 of SEQ ID NO: 9.

As described herein, a rAAVhu68 has a rAAVhu68 capsid produced in a production system expressing capsids from an AAVhu68 nucleic acid which encodes the vp1 amino acid sequence of SEQ ID NO: 9, and optionally additional nucleic acid sequences, e.g., encoding a vp 3 protein free of the vp1 and/or vp2-unique regions. The rAAVhu68 resulting from production using a single nucleic acid sequence vp1 produces the heterogenous populations of vp1 proteins, vp2 proteins and vp3 proteins. More particularly, the AAVhu68 capsid contains subpopulations within the vp1 proteins, within the vp2 proteins and within the vp3 proteins which have modifications from the predicted amino acid residues in SEQ ID NO: 9. These subpopulations include, at a minimum, deamidated asparagine (N or Asn) residues. For example, asparagines in asparagine-glycine pairs are highly deamidated.

In one embodiment, the AAVhu68 vp1 nucleic acid sequence has the sequence of SEQ ID NO: 8, or a strand complementary thereto, e.g., the corresponding mRNA or tRNA. In certain embodiments, the vp2 and/or vp3 proteins may be expressed additionally or alternatively from different nucleic acid sequences than the vp1, e.g., to alter the ratio of the vp proteins in a selected expression system. In certain embodiments, also provided is a nucleic acid sequence which encodes the AAVhu68 vp3 amino acid sequence of SEQ ID NO: 9 (about aa 203 to 736) without the vp1-unique region (about aa 1 to about aa 137) and/or vp2-unique regions (about aa 1 to about aa 202), or a strand complementary thereto, the corresponding mRNA or tRNA (about nt 607 to about nt 2211 of SEQ ID NO: 8). In certain embodiments, also provided is a nucleic acid sequence which encodes the AAVhu68 vp2 amino acid sequence of SEQ ID NO: 9 (about aa 138 to 736) without the vp1-unique region (about aa 1 to about 137), or a strand complementary thereto, the corresponding mRNA or tRNA (nt 412 to 2211 of SEQ ID NO:8).

However, other nucleic acid sequences which encode the amino acid sequence of SEQ ID NO: 9 may be selected for use in producing rAAVhu68 capsids. In certain embodiments, the nucleic acid sequence has the nucleic acid sequence of SEQ ID NO:8 or a sequence at least 70% to 99% identical, at least 75%, at least 80%, at least 85%, at least 90%, at least 95%, at least 97%, at least 99%, identical to SEQ ID NO: 8 which encodes SEQ ID NO: 9. In certain embodiments, the nucleic acid sequence has the nucleic acid sequence of SEQ ID NO: 8 or a sequence at least 70% to 99.%, at least 75%, at least 80%, at least 85%, at least 90%, at least 95%, at least 97%, at least 99%, identical to about nt 412 to about nt 2211 of SEQ ID NO: 8 which encodes the vp2 capsid protein (about aa 138 to 736) of SEQ ID NO: 9. In certain embodiments, the nucleic acid sequence has the nucleic acid sequence of about nt 607 to about nt 2211 of SEQ ID NO:8 or a sequence at least 70% to 99.%, at least 75%, at least 80%, at least 85%, at least 90%, at least 95%, at least 97%, at least 99%, identical to nt SEQ ID NO: 8 which encodes the vp3 capsid protein (about aa 203 to 736) of SEQ ID NO: 9.

In certain embodiments, the AAVhu68 capsid is produced using a nucleic acid sequence of SEQ ID NO: 8 or a sequence at least 70%, at least 75%, at least 80%, at least 85%, at least 90%, at least 95%, at least 97%, at least 99%, which encodes the vp1 amino acid sequence of SEQ ID NO: 9 with a modification (e.g., deamidated amino acid) as described herein. In certain embodiments, the vp1 amino acid sequence is reproduced in SEQ ID NO: 9.

As used herein when used to refer to vp capsid proteins, the term “heterogenous” or any grammatical variation thereof, refers to a population consisting of elements that are not the same, for example, having vp1, vp2 or vp3 monomers (proteins) with different modified amino acid sequences. SEQ ID NO: 9 provides the encoded amino acid sequence of the AAVhu68 vp1 protein. The term “heterogenous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified.

For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position (e.g., at least 80% of the asparagines at amino acid 57 based on the numbering of SEQ ID NO: 9 [AAVhu68] may be deamidated based on the total vp1 proteins may be deamidated based on the total vp1, vp2 and vp3 proteins). Such percentages may be determined using 2D-gel, mass spectrometry techniques, or other suitable techniques.

In the AAVhu68 capsid protein, 4 residues (N57, N329, N452, N512) routinely display levels of deamidation >70% and it most cases >90% across various lots. Additional asparagine residues (N94, N253, N270, N304, N409, N477, and Q599) also display deamidation levels up to ˜20% across various lots. The deamidation levels were initially identified using a trypsin digest and verified with a chymotrypsin digestion.

The AAVhu68 capsid contains subpopulations within the vp1 proteins, within the vp2 proteins and within the vp3 proteins which have modifications from the predicted amino acid residues in SEQ ID NO: 9. These subpopulations include, at a minimum, certain deamidated asparagine (N or Asn) residues. For example, certain subpopulations comprise at least one, two, three or four highly deamidated asparagines (N) positions in asparagine-glycine pairs in SEQ ID NO: 9 and optionally further comprising other deamidated amino acids, wherein the deamidation results in an amino acid change and other optional modifications.

In certain embodiments, a rAAV having an AAVrh91 capsid is provided. A nucleic acid sequence encoding the AAVrh91 capsid is provided in SEQ ID NO: 27 and the encoded amino acid sequence is provided in SEQ ID NO: 28. Provided herein is an rAAV comprising at least one of the vp1, vp2 and the vp3 of AAVrh91 (SEQ ID NO: 28). Also provided herein are rAAV comprising an AAV capsid encoded by at least one of the vp1, vp2 and the vp3 of AAVrh91 (SEQ ID NO: 27). In yet another embodiment, a nucleic acid sequence encoding the AAVrh91 amino acid sequence is provided in SEQ ID NO: 29 and the encoded amino acid sequence is provided in SEQ ID NO: 28. Also provided herein are rAAV comprising an AAV capsid encoded by at least one of the vp1, vp2 and the vp3 of AAVrh9leng (SEQ ID NO: 29). In certain embodiments, the vp1, vp2 and/or vp3 is the full-length capsid protein of AAVrh91 (SEQ ID NO: 28). In other embodiments, the vp1, vp2 and/or vp3 has an N-terminal and/or a C-terminal truncation (e.g. truncation(s) of about 1 to about 10 amino acids).

In certain embodiments, a rAAV is provided which comprises: (A) an AAVrh91 capsid comprising one or more of: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 28, vp1 proteins produced from SEQ ID NO: 27, or vp1 proteins produced from a nucleic acid sequence at least 70% identical to SEQ ID NO: 27 which encodes the predicted amino acid sequence of 1 to 736 of SEQ ID NO: 28, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 28, vp2 proteins produced from a sequence comprising at least nucleotides 412 to 2208 of SEQ ID NO: 27, or vp2 proteins produced from a nucleic acid sequence at least 70% identical to at least nucleotides 412 to 2208 of SEQ ID NO: 27 which encodes the predicted amino acid sequence of at least about amino acids 138 to 736 of SEQ ID NO: 28, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 28, vp3 proteins produced from a sequence comprising at least nucleotides 607 to 2208 of SEQ ID NO: 27, or vp3 proteins produced from a nucleic acid sequence at least 70% identical to at least nucleotides 607 to 2208 of SEQ ID NO: 27 which encodes the predicted amino acid sequence of at least about amino acids 203 to 736 of SEQ ID NO: 28; and/or (2) a heterogeneous population of vp1 proteins which are the product of a nucleic acid sequence encoding the amino acid sequence of SEQ ID NO: 28, a heterogeneous population of vp2 proteins which are the product of a nucleic acid sequence encoding the amino acid sequence of at least about amino acids 138 to 736 of SEQ ID NO: 28, and a heterogeneous population of vp3 proteins which are the product of a nucleic acid sequence encoding at least amino acids 203 to 736 of SEQ ID NO: 28 wherein: the vp1, vp2 and vp3 proteins contain subpopulations with amino acid modifications comprising at least two highly deamidated asparagines (N) in asparagine-glycine pairs in SEQ ID NO: 28 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In yet another embodiment, a recombinant adeno-associated virus rAAV is provided which comprises: (A) an AAVrh91 capsid comprising one or more of: (1) AAVrh91 capsid proteins comprising: a heterogeneous population of AAVrh91 vp1 proteins selected from: vp1 proteins produced by expression from a nucleic acid sequence which encodes the predicted amino acid sequence of 1 to 736 of SEQ ID NO: 28, vp1 proteins produced from SEQ ID NO:29, or vp1 proteins produced from a nucleic acid sequence at least 70% identical to SEQ ID NO: 28 which encodes the predicted amino acid sequence of 1 to 736 of SEQ ID NO: 28, a heterogeneous population of AAVrh91 vp2 proteins selected from: vp2 proteins produced by expression from a nucleic acid sequence which encodes the predicted amino acid sequence of at least about amino acids 138 to 736 of SEQ ID NO: 28, vp2 proteins produced from a sequence comprising at least nucleotides 412 to 2208 of SEQ ID NO: 29, or vp2 proteins produced from a nucleic acid sequence at least 70% identical to at least nucleotides 412 to 2208 of SEQ ID NO: 29 which encodes the predicted amino acid sequence of at least about amino acids 138 to 736 of SEQ ID NO: 28, a heterogeneous population of AAVrh91 vp3 proteins selected from: vp3 proteins produced by expression from a nucleic acid sequence which encodes the predicted amino acid sequence of at least about amino acids 203 to 736 of SEQ ID NO: 28, vp3 proteins produced from a sequence comprising at least nucleotides 607 to 2208 of SEQ ID NO: 29, or vp3 proteins produced from a nucleic acid sequence at least 70% identical to at least nucleotides 607 to 2208 of SEQ ID NO: 28 which encodes the predicted amino acid sequence of at least about amino acids 203 to 736 of SEQ ID NO: 28; and/or (2) a heterogeneous population of vp1 proteins which are the product of a nucleic acid sequence encoding the amino acid sequence of SEQ ID NO: 28, a heterogeneous population of vp2 proteins which are the product of a nucleic acid sequence encoding the amino acid sequence of at least about amino acids 138 to 736 of SEQ ID NO: 28, and a heterogeneous population of vp3 proteins which are the product of a nucleic acid sequence encoding at least amino acids 203 to 736 of SEQ ID NO: 28, wherein: the vp1, vp2 and vp3 proteins contain subpopulations with amino acid modifications comprising at least two highly deamidated asparagines (N) in asparagine-glycine pairs in SEQ ID NO: 28 and optionally further comprising subpopulations comprising other deamidated amino acids, wherein the deamidation results in an amino acid change; and (B) a vector genome in the AAVrh91 capsid, the vector genome comprising a nucleic acid molecule comprising AAV inverted terminal repeat sequences and a non-AAV nucleic acid sequence encoding a product operably linked to sequences which direct expression of the product in a host cell.

In certain embodiments, the rAAV provided has AAVrh91 vp1, vp2 and vp3 subpopulations with amino acid modifications comprising at least two highly deamidated asparagines (N) in asparagine-glycine pairs in SEQ ID NO: 28 and optionally further comprising subpopulations comprising other deamidated amino acids, wherein the deamidation results in an amino acid change. High levels of deamidation at N-G pairs N57, N383 and/or N512 are observed, relative to the number of SEQ ID NO: 28. In certain embodiments, AAVrh91 may have other residues deamidated, e.g., typically at less than 10% and/or may have other modifications, including phosphorylation (e.g., where present, in the range of about 2 to about 30%, or about 2 to about 20%, or about 2 to about 10%) (e.g., at S149), or oxidation (e.g, at one or more of —W22, —M211, W247, M403, M435, M471, W478, W503, —M537, —M541, —M559, —M599, M635, and/or, W695). Optionally the W may oxidize to kynurenine.

TABLE
AAVrh91 Deamidation

| AAVrh91 | |
|---|---|
| Deamidation based | |
| on VP1 numbering | % Deamidation |
| N57 + Deamidation | 65-90, 70-95, 80-95, 75-100, 80-100, or 90-100 |
| N94 + Deamidation | 2-15 or 2-5 |
| N303 + Deamidation | 2-15 or 5-10 |
| N383 + Deamidation | 65-90, 70-95, 80-95, 75-100, 80-100, or 90-100 |
| N497 + Deamidation | 2-15 or 5-10 |
| N512 + Deamidation | 65-90, 70-95, 80-95, 75-100, 80-100, or 90-100 |
| ~N691 + Deamidation | 2-15, 2-10, or 5-10 |

In certain embodiments, an AAVrh91 capsid is modified in one or more of the positions identified in the preceding table, in the ranges provided, as determined using mass spectrometry with a trypsin enzyme. In certain embodiments, one or more of the positions, or the glycine following the N is modified as described herein. Residue numbers are based on the AAVrh91 sequence provided herein. See, SEQ ID NO: 28.

In certain embodiments, an AAVrh91 capsid comprises: a heterogeneous population of vp1 proteins which are the product of a nucleic acid sequence encoding the amino acid sequence of SEQ ID NO: 28, a heterogeneous population of vp2 proteins which are the product of a nucleic acid sequence encoding the amino acid sequence of at least about amino acids 138 to 736 of SEQ ID NO: 28, and a heterogeneous population of vp3 proteins which are the product of a nucleic acid sequence encoding at least amino acids 203 to 736 of SEQ ID NO: 28.

In certain embodiments, the nucleic acid sequence encoding the AAVrh91 vp1 capsid protein is provided in SEQ ID NO: 27. In other embodiments, a nucleic acid sequence of 70% to 99.9% identity to SEQ ID NO: 27 may be selected to express the AAVrh91 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 27. However, other nucleic acid sequences which encode the amino acid sequence of SEQ ID NO: 28 may be selected for use in producing rAAV capsids. In certain embodiments, the nucleic acid sequence has the nucleic acid sequence of SEQ ID NO: 27 or a sequence at least 70% to 99.9% identical, at least 75%, at least 80%, at least 85%, at least 90%, at least 95%, at least 97%, or at least 99% identical to SEQ ID NO: 27 which encodes SEQ ID NO: 28. In certain embodiments, the nucleic acid sequence has the nucleic acid sequence of SEQ ID NO: 27 or a sequence at least 70% to 99.9%, at least 75%, at least 80%, at least 85%, at least 90%, at least 95%, at least 97%, or at least 99% identical to about nt 412 to about nt 2208 of SEQ ID NO: 27 which encodes the vp2 capsid protein (about aa 138 to 736) of SEQ ID NO: 28. In certain embodiments, the nucleic acid sequence has the nucleic acid sequence of about nt 607 to about nt 2208 of SEQ ID NO: 27 or a sequence at least 70% to 99.9%, at least 75%, at least 80%, at least 85%, at least 90%, at least 95%, at least 97%, or at least 99% identical to nt 607 to about nt 2208 SEQ ID NO: 27 which encodes the vp3 capsid protein (about aa 203 to 736) of SEQ ID NO: 28.

In certain embodiments, the nucleic acid sequence encoding the AAVrh91 vp1 capsid protein is provided in SEQ ID NO: 29. In other embodiments, a nucleic acid sequence of 70% to 99.9% identity to SEQ ID NO: 29 may be selected to express the AAVrh91 capsid proteins. In certain other embodiments, the nucleic acid sequence is at least about 75% identical, at least 80% identical, at least 85%, at least 90%, at least 95%, at least 97% identical, or at least 99% to 99.9% identical to SEQ ID NO: 29. However, other nucleic acid sequences which encode the amino acid sequence of SEQ ID NO: 28 may be selected for use in producing rAAV capsids. In certain embodiments, the nucleic acid sequence has the nucleic acid sequence of SEQ ID NO: 29 or a sequence at least 70% to 99.9% identical, at least 75%, at least 80%, at least 85%, at least 90%, at least 95%, at least 97%, or at least 99% identical to SEQ ID NO: 29 which encodes SEQ ID NO: 28. In certain embodiments, the nucleic acid sequence has the nucleic acid sequence of SEQ ID NO: 29 or a sequence at least 70% to 99.9%, at least 75%, at least 80%, at least 85%, at least 90%, at least 95%, at least 97%, or at least 99% identical to about nt 412 to about nt 2208 of SEQ ID NO: 29 which encodes the vp2 capsid protein (about aa 138 to 736) of SEQ ID NO: 28. In certain embodiments, the nucleic acid sequence has the nucleic acid sequence of about nt 607 to about nt 2208 of SEQ ID NO: 29 or a sequence at least 70% to 99.9%, at least 75%, at least 80%, at least 85%, at least 90%, at least 95%, at least 97%, or at least 99% identical to nt 607 to about nt 2208 SEQ ID NO: 29 which encodes the vp3 capsid protein (about aa 203 to 736) of SEQ ID NO: 28.

The invention also encompasses nucleic acid sequences encoding the AAVrh91 capsid sequence (SEQ ID NO: 28) or a mutant AAVrh91, in which one or more residues has been altered in order to decrease deamidation, or other modifications which are identified herein. Such nucleic acid sequences can be used in production of mutant AAVrh91 capsids.

In certain embodiments, the rAAV as described herein is a self-complementary AAV. The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In certain embodiments, the rAAV described herein is nuclease-resistant. Such nuclease may be a single nuclease, or mixtures of nucleases, and may be endonucleases or exonucleases. A nuclease-resistant rAAV indicates that the AAV capsid has fully assembled and protects these packaged genomic sequences from degradation (digestion) during nuclease incubation steps designed to remove contaminating nucleic acids which may be present from the production process. In many instances, the rAAV described herein is DNase resistant.

It should be understood that the compositions in the rAAV described herein are intended to be applied to other compositions, regiments, aspects, embodiments, and methods described across the Specification.

Production of rAAV.hIDUA Viral Particles

The invention provides for the manufacture of the rAAV.hIDUA pharmaceutical compositions and formulations described herein. Methods for manufacturing the gene therapy vectors described herein include methods well known in the art such as generation of plasmid DNA used for production of the gene therapy vectors, generation of the vectors, and purification of the vectors.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed by anion exchange resin chromatography are used to purify the vector drug product and to remove empty capsids. These methods are described in more detail in WO 2017/160360 entitled “Scalable Purification Method for AAV9”, which is incorporated by reference herein. In brief, the method for separating rAAV9 particles having packaged genomic sequences from genome-deficient AAV9 intermediates involves subjecting a suspension comprising recombinant AAV9 viral particles and AAV 9 capsid intermediates to fast performance liquid chromatography, wherein the AAV9 viral particles and AAV9 intermediates are bound to a strong anion exchange resin equilibrated at a pH of 10.2, and subjected to a salt gradient while monitoring eluate for ultraviolet absorbance at about 260 and about 280. Although less optimal for rAAV9, the pH may be in the range of about 10.0 to 10.4. In this method, the AAV9 full capsids are collected from a fraction which is eluted when the ratio of A260/A280 reaches an inflection point. In one example, for the Affinity Chromatography step, the diafiltered product may be applied to a Capture Select′ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr. 15; 20 (R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95(6):1081-92; SAMI S. THAKUR, Production of Recombinant Adeno-associated viral vectors in yeast. Thesis presented to the Graduate School of the University of Florida, 2012; Kondratov 0 et al. Direct Head-to-Head Evaluation of Recombinant Adeno-associated Viral Vectors Manufactured in Human versus Insect Cells, Mol Ther. 2017 Aug. 10. pii: S1525-0016 (17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28(1):15-22. doi: 10.1089/hgtb.2016.164.; Li L et. al.. Production and characterization of novel recombinant adeno-associated virus replicative-form genomes: a eukaryotic source of DNA for gene transfer. PLoS One. 2013 Aug. 1; 8 (8):e69879. doi: 10.1371/journal.pone.0069879. Print 2013; Galibert L et al, Latest developments in the large-scale production of adeno-associated virus vectors in insect cells toward the treatment of neuromuscular diseases. J Invertebr Pathol. 2011 July; 107 Suppl:580-93. doi: 10.1016/j.jip.2011.05.008; and Kotin R M, Large-scale recombinant adeno-associated virus production. Hum Mol Genet. 2011 Apr. 15; 20 (R1):R2-6. doi: 10.1093/hmg/ddr141. Epub 2011 Apr. 29.

Conventional methods for characterization or quantification of rAAV are available to one of skill in the art. To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Viral. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e. SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad-spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Methods for determining the ratio among vp1, vp2 and vp3 of capsid protein are also available. See, e.g., Vamseedhar Rayaprolu et al, Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

It should be understood that the method for production of rAAV described herein are intended to be applied to other compositions, regiments, aspects, embodiments, and methods described across the Specification

### Pharmaceutical Compositions and Formulations

In certain embodiments, provided herein is a pharmaceutical composition comprising a vector, such as a rAAV, as described herein in a formulation buffer. In certain embodiments, the pharmaceutical composition is suitable for co-administering with a functional hIDUA protein (ERT) (e.g. Aldurazyme® (laronidase); Sanofi Genzyme). In one embodiment, provided is a pharmaceutical composition comprising a rAAV as described herein in a formulation buffer. In certain embodiments, the rAAV is formulated at about 1×109 genome copies (GC)/mL to about 1×1014 GC/mL. In a further embodiment, the rAAV is formulated at about 3×109 GC/mL to about 3×1013 GC/mL. In yet a further embodiment, the rAAV is formulated at about 1×109 GC/mL to about 1×1013 GC/mL. In one embodiment, the rAAV is formulated at least about 1×1011 GC/mL.

In certain embodiments, the pharmaceutical composition comprises an expression cassette having an hIDUA coding sequence in a non-viral vector system. This may include, e.g, naked DNA, naked RNA, an inorganic particle, a lipid or lipid-like particle, a chitosan-based formulation and others known in the art. Such a non-viral vector system may include, e.g., a plasmid or non-viral genetic element, or a protein-based vector.

In certain embodiments, the pharmaceutical composition comprises a non-replicating viral vector. Suitable viral vectors may include any suitable delivery vector, such as, e.g., a recombinant adenovirus, a recombinant lentivirus, a recombinant bocavirus, a recombinant adeno-associated virus (AAV), or another recombinant parvovirus. In certain embodiments, the viral vector is a recombinant AAV for delivery of a hIDUA to a patient in need thereof.

In one embodiment, the pharmaceutical composition comprises a vector that includes an expression cassette comprising an hIDUA coding sequence, and a formulation buffer suitable for delivery via intracerebroventricular (ICV), intrathecal (IT), intracisternal or intravenous (IV) injection. In one embodiment, the expression cassette comprising the hIDUA coding sequence is in packaged a recombinant AAV. In one embodiment, the pharmaceutical composition comprises a functional hIDUA polypeptide, or a functional fragment thereof, for delivery to a subject as an enzyme replacement therapy (ERT). Such pharmaceutical compositions are usually administered intravenously, however intradermal, intramuscular, or oral administration is also possible in some circumstances. The compositions can be administered for prophylactic treatment of individuals suffering from, or at risk of, MPSI, Hurler, Hurler-Scheie and/or Scheie syndromes. For therapeutic applications, the pharmaceutical compositions are administered to a patient suffering from established disease in an amount sufficient to reduce the concentration of accumulated metabolite and/or prevent or arrest further accumulation of metabolite. For individuals at risk of lysosomal enzyme deficiency disease, the pharmaceutical compositions are administered prophylactically in an amount sufficient to either prevent or inhibit accumulation of metabolite. The pharmaceutical compositions comprising an hIDUA protein described herein are administered in a therapeutically effective amount. In general, a therapeutically effective amount can vary depending on the severity of the medical condition in the subject, as well as the subject's age, general condition, and gender. Dosages can be determined by the physician and can be adjusted as necessary to suit the effect of the observed treatment. In one aspect, provided herein is a pharmaceutical composition for ERT formulated to contain a unit dosage of a hIDUA protein, or functional fragment thereof.

In certain embodiments, the formulation further comprises a surfactant, preservative, excipients, and/or buffer dissolved in the aqueous suspending liquid. In one embodiment, the buffer is PBS. In another embodiment, the buffer is an artificial cerebrospinal fluid (aCSF), e.g., Eliott's formulation buffer; or Harvard apparatus perfusion fluid (an artificial CSF with final Ion Concentrations (in mM): Na 150; K 3.0; Ca 1.4; Mg 0.8; P 1.0; Cl 155). Various suitable solutions are known including those which include one or more of: buffering saline, a surfactant, and a physiologically compatible salt or mixture of salts adjusted to an ionic strength equivalent to about 100 mM sodium chloride (NaCl) to about 250 mM sodium chloride, or a physiologically compatible salt adjusted to an equivalent ionic concentration.

Suitably, the formulation is adjusted to a physiologically acceptable pH, e.g., in the range of pH 6 to 8, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of 6.8 to about 7.2 may be desired. However, other pHs within the broadest ranges and these subranges may be selected for other route of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol.

In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In one example, the formulation may contain, e.g., buffered saline solution comprising one or more of sodium chloride, sodium bicarbonate, dextrose, magnesium sulfate (e.g., magnesium sulfate 7H2O), potassium chloride, calcium chloride (e.g., calcium chloride 2H2O), dibasic sodium phosphate, and mixtures thereof, in water. Suitably, for intrathecal delivery, the osmolarity is within a range compatible with cerebrospinal fluid (e.g., about 275 to about 290); see, e.g., emedicine.medscape.com/article/2093316-overview. Optionally, for intrathecal delivery, a commercially available diluent may be used as a suspending agent, or in combination with another suspending agent and other optional excipients. See, e.g., Elliotts solution [Lukare Medical].

In certain embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA

In one embodiment, a frozen composition which contains an rAAV in a buffer solution as described herein, in frozen form, is provided. Optionally, one or more surfactants (e.g., Pluronic F68), stabilizers or preservatives is present in this composition. Suitably, for use, a composition is thawed and titrated to the desired dose with a suitable diluent, e.g., sterile saline or a buffered saline.

In certain embodiments, a kit is provided which includes a concentrated vector suspended in a formulation (optionally frozen), optional dilution buffer, and devices and other components required for intrathecal administration are provided. In another embodiment, the kit may additional or alternatively include components for intravenous delivery. In one embodiment, the kit provides sufficient buffer to allow for injection. Such buffer may allow for about a 1:1 to a 1:5 dilution of the concentrated vector, or more. In other embodiments, higher or lower amounts of buffer or sterile water are included to allow for dose titration and other adjustments by the treating clinician. In still other embodiments, one or more components of the device are included in the kit.

In certain embodiments, provided herein is a pharmaceutical composition comprising a vector, such as a rAAV, as described herein and a pharmaceutically acceptable carrier. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Delivery vehicles such as liposomes, nanocapsules, microparticles, microspheres, lipid particles, vesicles, and the like, may be used for the introduction of the compositions of the present invention into suitable host cells.

In particular, the rAAV vector may be formulated for delivery either encapsulated in a lipid particle, a liposome, a vesicle, a nanosphere, or a nanoparticle or the like. In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

In one aspect, provided herein is a pharmaceutical composition comprising a viral vector (e.g. rAVV) as described herein in a formulation buffer. In certain embodiments, the compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰ 3×10¹⁰ 4×10¹⁰, 5×10¹⁰, 6×10¹⁰, 7×10¹⁰, 8×10¹⁰ , or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10° GC per dose including all integers or fractional amounts within the range. In another embodiment, the compositions are formulated to contain at least 1×10¹⁴, 2×10¹⁴, 3×10¹⁴ 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In one embodiment, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL. In one embodiment, the pharmaceutical composition comprising a rAAV as described herein is administrable at a dose of about 1×10⁹ GC per gram of brain mass to about 1×10¹⁴ GC per gram of brain mass.

In certain embodiments, the composition may be formulated in a suitable aqueous suspension media (e.g., a buffered saline) for delivery by any suitable route. The compositions provided herein are useful for systemic delivery of high doses of viral vector. For rAAV, a high dose may be at least 1×10¹³ GC or at least 1×10¹⁴ GC.

However, for improved safety, the miRNA sequences provided herein may be included in expression cassettes and/or vector genomes which are delivered at other lower doses. The aqueous suspension or pharmaceutical compositions described herein are designed for delivery to subjects in need thereof by any suitable route or a combination of different routes. In one embodiment, the pharmaceutical composition is formulated for delivery via intracerebroventricular (ICV), intrathecal (IT), or intracisternal injection. In one embodiment, the compositions described herein are designed for delivery to subjects in need thereof by intravenous (IV) injection. Alternatively, other routes of administration may be selected (e.g., oral, inhalation, intranasal, intratracheal, intraarterial, intraocular, intramuscular, and other parenteral routes). In certain embodiments, the composition is delivered by two different routes at essentially the same time.

As used herein, the terms “intrathecal delivery” or “intrathecal administration” refer to a route of administration for drugs via an injection into the spinal canal, more specifically into the subarachnoid space so that it reaches the cerebrospinal fluid (CSF).

Intrathecal delivery may include lumbar puncture, intraventricular, suboccipital/intracisternal, and/or C1-2 puncture. For example, material may be introduced for diffusion throughout the subarachnoid space by means of lumbar puncture. In another example, injection may be into the cisterna magna. Intracisternal delivery may increase vector diffusion and/or reduce toxicity and inflammation caused by the administration. See, e.g., Christian Hinderer et al., Widespread gene transfer in the central nervous system of cynomolgus macaques following delivery of AAV9 into the cisterna magna, Mol Ther Methods Clin Dev. 2014; 1: 14051. Published online 2014 Dec. 10. doi: 10.1038/mtm.2014.51.

As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube.

It should be understood that the compositions in the pharmaceutical compositions and formulations described herein are intended to be applied to other compositions, regimens, aspects, embodiments and methods described across the Specification.

Methods of Treatment

Provided herein are methods for MPSI, Hurler, Hurler-Scheie and/or Scheie syndrome comprising delivering a therapeutically effective amount of a hIDUA as described. In particular, provided herein are methods for preventing, treating, and/or ameliorating neurocognitive decline in a patient diagnosed with MPSI, Hurler, Hurler-Scheie and/or Scheie syndrome, comprising delivering a therapeutically effective amount of a rAAV.hIDUA described herein to a patient in need thereof. A therapeutically effective amount of the rAAV.hIDUA vector described herein may correct one or more of the symptoms identified in any one of the following paragraphs.

Methods for sequencing a protein, peptide, or polypeptide (e.g., as an immunoglobulin) are known to those of skill in the art. Once the sequence of a protein is known, there are web-based and commercially available computer programs, as well as service based companies which back translate the amino acids sequences to nucleic acid coding sequences. See, e.g., backtranseq by EMBOSS, available at www.ebi.ac.uk/Tools/st/; Gene Infinity, available at geneinfinity.org/sms/sms_backtranslation.html); ExPasy, available at expasy.org/tools/. In one embodiment, the RNA and/or cDNA coding sequences are designed for optimal expression in human cells.

In certain embodiments, the compositions provided herein are useful for delivery of a desired transgene product to patient, while for repressing transgene expression in dorsal root ganglion neurons. In certain embodiments, the compositions provided herein are useful for a method for modulating neuronal degeneration and/or decrease secondary dorsal spinal cord axonal degeneration following intrathecal or systemic gene therapy administration. Thus, while the compositions provided herein are particularly useful for delivery of gene therapy to the CNS, they may also be useful for other routes of delivery, including e.g. systemic IV delivery, where high doses of the gene therapy may result in DRG transduction and toxicity. The method involves delivering a composition comprising an expression cassette or vector genome comprising the transgene and miRNA target(s) to a patient.

In certain embodiments, the method comprises delivering an expression cassette or vector genome that includes a miR-183 target sequence to repress transgene expression levels in the DRG. In certain embodiments, the method comprises delivering an expression cassette or vector genome useful for repressing transgene expression in the DRG, wherein the expression cassette or vector genome includes at least two miR183 target sequences, at least three miR183 target sequences, at least four miR183 target sequences, at least five miR183 target sequences, at least six miR183 target sequences, at least seven miR183 target sequences, or at least eight miR183 target sequences. In certain embodiments, the method comprises delivering an expression cassette or vector genome useful for repressing transgene expression in the DRG, wherein the expression cassette or vector genome comprises eight miR183 target sequences. In certain embodiments, the method enhances expression in one or more cells present in the CNS selected from one or more of pyramidal neurons, purkinje neurons, granule cells, spindle neurons, interneuron cells, astrocytes, oligodendrocytes, microglia, and/or ependymal cells.

In certain embodiments, provided is a method useful for delivering and/or enhancing expression of transgene in lower motor neurons the retina, inner ear, and olfactory receptors comprising delivering an expression cassette or vector genome that includes a transgene operably linked to one or more miR-183 target sequences and/or more miR-183 target sequences. In certain embodiments, the cells or tissues may be one or more of liver, or heart.

In yet another embodiment, provided is a method comprising delivering an expression cassette or vector genome to cells present in the CNS wherein the expression cassette or vector genome comprises one or more miR-183 target sequences and lacks a transgene (i.e. a sequence encoding a heterologous gene product). In such embodiments, delivery of miR-183 to cells of the CNS is achieved. In certain embodiments, delivery of an expression cassette or vector genome comprising miR-183 sequences results in repression of DRG expression and enhanced gene expression in certain other cells present in the CNS.

In certain embodiments, the compositions provided herein are useful in methods for enhancing expression of a transgene in a cell outside the CNS. In certain embodiments, methods for enhancing expression in a cell outside the CNS comprise delivering an expression cassette or vector genome that includes a miR-182 target sequence to a patient.

In one embodiment, the suspension has a pH of about 6.8 to about 7.32.

Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In one embodiment, the composition comprising an rAAV as described herein is administrable at a dose of about 1×10⁹ GC per gram of brain mass to about 1×10¹⁴ GC per gram of brain mass. In certain embodiments, the rAAV is co-administered systemically at a dose of about 1×10⁹ GC per kg body weight to about 1×10¹³ GC per kg body weight

In certain embodiments, the subject is administered a therapeutically effective amount of a composition comprising a nucleic acid sequence encoding an hIDUA gene product and miRNA target sequences, which delivers and expresses hIDUA in target cells and which specifically detargets DRG expression.

In certain embodiments, an AAV.alpha-L-iduronidase (AAV.IDUA) gene therapy vector comprises a vector genome comprising at least one, at least two, at least three, or at least four miR target sequences of the miRNA183 cluster (including miR-183, miR-182, and miR183 target sequences, or combinations thereof) operably linked to the coding sequence for the IDUA gene (see, e.g., nt 1943-3901 of SEQ ID NO: 14). In certain embodiments, the vector genome comprises multiple copies of the same miR target sequence each separated by a spacer which may be the same or which may differ from each other. In another embodiment, the vector genome comprises three to six copies of a miR183 cluster target sequence, optionally wherein one or more of the target sequences is at least about 80% to about 99% complementarity to a miR-183 cluster member. In another embodiment, the vector comprises one, two, three, or four copies of a miR183 target sequence. Such a vector genome may optionally contain additional target sequences that correspond to members of the miR183 cluster. In certain embodiments, the vector genome contains a single miR target sequence for a miR183 cluster member. In certain embodiments, the vector genome contains two miR target sequences for miR183 cluster members and optionally at least one spacer. In certain embodiments, the vector contains three miR target sequences for miR183 cluster members and optionally at least two spacers. In certain embodiments, the vector genome contains two or more miR target sequences for the miR183 cluster which differ in sequence from one another. In certain embodiments, the vector genomes described herein are carried by a non-AAV vector.

In one embodiment, the expression cassette is in a vector genome delivered in an amount of about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10¹¹ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹³ GC/g, about 9.5×10¹³ GC/g, or about 1.0×10¹⁴ GC/g brain mass.

In certain embodiments, the miR target sequence-containing compositions provided herein minimize the dose, duration, and/or amount of immunosuppressive co-therapy required by the patient. Currently, immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-γ, an opioid, or TNF-α (tumor necrosis factor-alpha) binding agent. In certain embodiments, the immunosuppressive therapy may be started 0, 1, 2, 7, or more days prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. In certain embodiments, the miR target sequence-containing compositions provided herein eliminate the need for immunosuppressive therapy prior to, during, or following delivery of a gene therapy (e.g., rAAV) vector.

In one embodiment, a composition comprising the expression cassette as described herein is administrated once to the subject in need. In certain embodiments, the expression cassette is delivered via an rAAV.

Patients who are candidates for treatment are pediatric and adult patients with MPSI and/or the symptoms associated with Hurler, Hurler-Scheie and Scheie. MPSI disorders are a spectrum of disease from early severe (Hurler) to later onset (Scheie) forms. Hurler syndrome is typically characterized by no (0%) IDUA enzyme activity and diagnosed early and is characterized by developmental delay, hepatospenomegaly, skeletal involvement, corneal clouding, joint involvement, deafness, cardiac involvement, and death during the first decade of life. Hurler-Scheie patients have been observed to have some IDUA enzyme activity (greater than 0% but typically less than 2%) and by having variable intellectual effects, respiratory disease, obstructive airway disease, cardiovascular disease, joint stiffness/contractures, skeletal abnormalities, decreased visual acuity, and death in teens or twenties. Patients with Scheie syndrome typically have at least 2% of “normal” IDUA enzyme activity, and are diagnosed later; such patients typically have normal intelligence, but have hepatosplenomegaly, joint involvement, nerve entrapment, deafness, cardiac involvement, and a normal life span. See, also, Newborn Screening for Mucopolysaccharidosis Type 1 (MPS I): A Systematic Review of Evidence Report of Final Findings, Final Version 1.1, Prepared for: MATERNAL AND CHILD HEALTH BUREAU.

The compositions provided herein avoid complications of long-term enzyme replacement therapy (ERT) related to immune response to the recombinant enzyme, which can range from mild to full-blown anaphylaxis as well as complications of life-long peripheral access such as local and systemic infections. In contrast to ERT, the composition of the invention does not require life-long, repeated weekly injections.

Without wishing to be bound by theory, the therapeutic method described herein is believed to be useful for correcting at least the central nervous system phenotype associated with MPSI disorders by providing efficient, long-term gene transfer afforded by vectors with high transduction efficiency which provide continuous, elevated circulating IDUA levels, which provides therapeutic leverage outside the CNS compartment. In addition, provided herein are methods for providing active tolerance and preventing antibody formation against the enzyme by a variety of routes, including by direct systemic delivery of the enzyme in protein form or in the form of rAAV.hIDUA prior to AAV-mediated delivery into CNS.

In some embodiments, patients diagnosed with Hurler syndrome are treated in accordance with the methods described herein. In some embodiments, patients diagnosed with Hurler-Scheie syndrome are treated in accordance with the methods described herein. In some embodiments, patients diagnosed with Scheie syndrome are treated in accordance with the methods described herein. In some embodiments, pediatric subjects with MPS I who have neurocognitive deficit are treated in accordance with the methods described herein.

In certain embodiments, newborn babies (3 months old or younger) are treated in accordance with the methods described herein. In certain embodiments, babies that are 3 months old to 9 months old are treated in accordance with the methods described herein. In certain embodiments, children that are 9 months old to 36 months old are treated in accordance with the methods described herein. In certain embodiments, children that are 3 years old to 12 years old are treated in accordance with the methods described herein. In certain embodiments, children that are 12 years old to 18 years old are treated in accordance with the methods described herein. In certain embodiments, adults that are 18 years old or older are treated in accordance with the methods described herein. In one embodiment, a patient may have Hurler syndrome and is a male or female of at least about 3 months to less than 12 months of age. In another embodiment, a patient may be male or female Hurler-Scheie patient and be at least about 6 years to up to 18 years of age. In other embodiments, the subjects may be older or younger, and may be male or female.

Suitably, patients selected for treatment may include those having one or more of the following characteristics: a documented diagnosis of MPS I confirmed by the lacking or diminished IDUA enzyme activity as measured in plasma, fibroblasts, or leukocytes; documented evidence of early-stage neurocognitive deficit due to MPS I, defined as either of the following, if not explainable by any other neurological or psychiatric factors:—A score of 1 standard deviation below mean on IQ testing or in 1 domain of neuropsychological function (language, memory, attention or non-verbal ability), OR-Documented historical evidence of a decline of greater than 1 standard deviation on sequential testing. Alternatively, increased GAGs in urine or genetic tests may be used. Prior to treatment, subjects, e.g., infants, preferably undergo genotyping to identify MPS I patients, i.e., patients that have mutations in the gene encoding hIDUA. Prior to treatment, the MPS I patient can be assessed for neutralizing antibodies (Nab) to the AAV serotype used to deliver the hIDUA gene. In certain embodiments, MPS I patients with neutralizing antibody titers to AAV that are less than or equal to 5 are treated in accordance with any one or more of the methods described herein.

Prior to treatment, the MPSI patient can be assessed for neutralizing antibodies (Nab) to the capsid of the AAV vector used to deliver the hIDUA gene. Such Nabs can interfere with transduction efficiency and reduce therapeutic efficacy. MPS I patients that have a baseline serum Nab titer 1:5 are good candidates for treatment with the rAAV.hIDUA gene therapy protocol. Treatment of MPS I patients with titers of serum Nab >1:5 may require a combination therapy, such as transient co-treatment with an immunosuppressant before and/or during treatment with rAAV.hIDUA vector delivery.

Optionally, immunosuppressive co-therapy may be used as a precautionary measure without prior assessment of neutralizing antibodies to the AAV vector capsid and/or other components of the formulation. Prior immunosuppression therapy may be desirable to prevent potential adverse immune reaction to the hIDUA transgene product, especially in patients who have virtually no levels of IDUA activity, where the transgene product may be seen as “foreign.” Results of non-clinical studies in mice, dogs and NHPs described infra are consistent with the development of an immune response to hIDUA and neuroinflammation. While a similar reaction may not occur in human subjects, as a precaution immunosuppression therapy is recommended for all recipients of rAAV.hIDUA.

Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected.

Nevertheless, in one embodiment, patients having one or more of the following characteristics may be excluded from treatment at the discretion of their caring physician:
Review of baseline MRI testing shows a contraindication for an IC injection.
History of prior head/neck surgery, which resulted in a contraindication to IC injection.
Has any contraindication to CT (or contrast) or to general anesthesia.
Has any contraindication to MRI (or gadolinium).
Has estimated glomerular filtration rate (eGFR)<30 mL/min/1.73 m2.
Has any neurocognitive deficit not attributable to MPS I or diagnosis of a neuropsychiatric condition.
Has any history of a hypersensitivity reaction to sirolimus, MMF, or prednisolone.
Has any condition that would not be appropriate for immunosuppressive therapy (e.g., absolute neutrophil count <1.3×10³/μL, platelet count <100×10³/μL, and hemoglobin <12 g/dL [male] or <10 g/dL [female]).
Has any contraindication to lumbar puncture.
Has undergone HSCT.
Has received laronidase via IT administration within 6 months prior to treatment.
Has received IT laronidase at any time and experienced a significant adverse event considered related to IT administration that would put the patient at undue risk.
Any history of lymphoma or history of another cancer, other than squamous cell or basal cell carcinoma of the skin, that has not been in full remission for at least 3 months before treatment.
Alanine aminotransferase (ALT) or aspartate aminotransferase (AST)>3×upper limit of normal (ULN) or total bilirubin >1.5×ULN, unless the patient has a previously known history of Gilbert's syndrome and a fractionated bilirubin that shows conjugated bilirubin <35% of total bilirubin.
History of human immunodeficiency virus (HIV)-positive test, history of active or recurrent hepatitis B or hepatitis C, or positive screening tests for hepatitis B, hepatitis C, or HIV.
Is pregnant, <6 weeks post-partum, breastfeeding, or planning to become pregnant (self or partner)
History of alcohol or substance abuse within 1 year before treatment.
Has a serious or unstable medical or psychological condition that, would compromise the patient's safety.
Uncontrolled seizures.

In other embodiments, a caring physician may determine that the presence of one or more of these physical characteristics (medical history) should not preclude treatment as provided herein.

It should be understood that the compositions in the methods described herein are intended to be applied to other compositions, regiments, aspects, embodiments and methods described across the Specification.

### Dosages & Mode of Administration

Pharmaceutical compositions suitable for administration to patients comprise a suspension of rAAV.hIDUA vectors in a formulation buffer comprising a physiologically compatible aqueous buffer, a surfactant and optional excipients. In certain embodiments, a pharmaceutical composition described herein is administered intrathecally. In other embodiments, a pharmaceutical composition described herein is administered intracisternally. In other embodiments, a pharmaceutical composition described herein is administered intravenously. In certain embodiments, the pharmaceutical composition is delivered via a peripheral vein by infusion over 20 minutes (±5 minutes). However, this time may be adjusted as needed or desired. However, still other routes of administration may be selected. Alternatively, or additionally, routes of administration may be combined, if desired.

While a single administration of the rAAV is anticipated to be effective, administration may be repeated (e.g., quarterly, bi-annually, annually, or as otherwise needed, particularly in treatment of newborns. Optionally, an initial dose of a therapeutically effective amount may be delivered over split infusion/injection sessions, taking into consideration the age and ability of the subject to tolerate infusions/injections. However, repeated weekly injections of a full therapeutic dose are not required, providing an advantage to the patient in terms of both comfort and therapeutic outcome.

In some embodiments, the rAAV suspension has an rAAV Genome Copy (GC) titer that is at least 1×10⁹ GC/mL. In certain embodiments, the rAAV Empty/Full particle ratio in the rAAV suspension is between 0.01 and 0.05 (95%-99% free of empty capsids). In some embodiments, an MPS I patient in need thereof is administered a dose of at least about 4×10⁸ GC/g brain mass to about 4×10¹¹ GC/g brain mass of the rAAV suspension.

The following therapeutically effective flat doses of rAAV.hIDUA can be administered to MPS I patients of the indicated age group:
Newborns: about 3.8×10¹² to about 1.9×10¹⁴ GC;
3—9 months: about 6×10¹² to about 3×10¹⁴ GC;
9-36 months: about 10¹³ to about 5×10¹⁴ GC;
3-12 years: about 1.2×10¹³ to about 6×10¹⁴ GC;
12+ years: about 1.4×10¹³ to about 7.0×10¹⁴ GC;
18+ years (adult): about 1.4×10¹³ to about 7.0×10¹⁴ GC.

In some embodiments, the dose administered to a 12+ year old MPS I patient (including 18+ year old) is 1.4×10¹³ genome copies (GC) (1.1×10¹⁰ GC/g brain mass). In some embodiments, the dose administered to a 12+ year old MPS I patient (including 18+ year old) is 7×10¹³ GC (5.6×10¹⁰ GC/g brain mass). In still a further embodiment, the dose administered to an MPSI patient is at least about 4×10⁸ GC/g brain mass to about 4×10¹¹ GC/g brain mass. In certain embodiments, the dose administered to MPS I newborns ranges from about 1.4×10¹¹ to about 1.4×10¹⁴ GC; the dose administered to infants 3-9 months ranges from about 2.4×10¹¹ to about 2.4×10¹⁴ GC; the dose administered to MPS I children 9-36 months ranges: about 4×10¹¹ to about 4×10¹⁴ GC; the dose administered to MPS I children 3-12 years: ranges from about 4.8×10¹¹ to about 4.8×10¹⁴ GC; the dose administered to children and adults 12+ years ranges from about 5.6×10¹¹ to about 5.6×10¹⁴ GC.

Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In one embodiment for intrathecal delivery, the patients are adult subjects and the dose comprises about 1×10⁸ GC to 5×10¹⁴ GC. In another embodiment, the dose comprises about 3.8×10¹² to about 1.9×10¹⁴ GC. In a further embodiment, the patients are infant subjects of at least about 3 months to up to 12 months of age having Hurler syndrome and the dose comprises at least the equivalent of 4×10⁸ GC rAAV.hIDUA/g brain mass to 3×10¹² GC rAAV.hIDUA/g brain mass. In another example, the patients are children of at least about 6 years to up to 18 years of age having Hurler-Scheie syndrome and the dose comprises the equivalent of at least 4×10⁸ GC rAAV.hIDUA/g brain mass to 3×10¹² GC rAAV.hIDUA/g brain mass.

### Monitoring Efficacy

Efficacy of the therapy can be measured by assessing (a) the prevention of neurocognitive decline in patients with MPSI; and (b) reductions in biomarkers of disease, e.g., GAG levels and/or enzyme activity in the CSF, serum and/or urine, and/or liver and spleen volumes. Neurocognition can be determined by measuring intelligence quotient (IQ), e.g., as measured by Bayley's Infantile Development Scale for Hurler subjects or as measured by the Wechsler Abbreviated Scale of Intelligence (WASI) for Hurler-Scheie subjects. Other appropriate measures of neurocognitive development and function may be utilized, e.g., assessing developmental quotient (DQ) using Bayley Scales of Infant Development (BSID-III), assessing memory using the Hopkins Verbal Learning Test, and/or using Tests of Variables of Attention (TOVA). Other neuropsychological function, such as vineland adaptive behavior scales, visual processing, fine motor, communication, socialization, daily living skills, and emotional and behavioral health are monitored. Magnetic Resonance Imaging (MRI) of brain to acquire volumetric, diffusion tensor imaging (DTI), and resting state data, median nerve cross-sectional area by ultrasonography, improvement in spinal cord compression, safety, liver size and spleen size are also administered.

Optionally, other measures of efficacy may include evaluation of biomarkers (e.g., polyamines as described herein) and clinical outcomes. Urine is evaluated for total GAG content, concentration of GAG relative to creatinine, as well as MPS I specific pGAGs. Serum and/or plasma is evaluated for IDUA activity, anti-IDUA antibodies, pGAG, and concentration of the heparin cofactor II-thrombin complex and markers of inflammation. CSF is evaluated for IDUA activity, anti-IDUA antibodies, hexosaminidase (hex) activity, and pGAG (such as heparan sulfate and dermatan sulfate). The presence of neutralizing antibodies to vector and binding antibodies to anti-IDUA antibodies may be assessed in CSF and serum. T-cell response to vector capsid or the hIDUA transgene product may be assessed by ELISPOT assay. Pharmacokinetics of IDUA expression in CSF, serum, and urine as well as vector concentration may also be monitored.

Combinations of gene therapy delivery of the rAAV.hIDUA to the CNS accompanied by systemic delivery of hIDUA are encompassed by the methods of the invention. Systemic delivery can be accomplished using ERT (e.g., using Aldurazyme®), or additional gene therapy using an rAAV.hIDUA with tropism for the liver (e.g., an rAAV.hIDUA bearing an AAV68 capsid).

Additional measures of clinical efficacy associated with systemic delivery may include, e.g., Orthopedic Measures, such as bone mineral density, bone mineral content, bone geometry and strength, Bone Density measured by dual energy x-ray absorptiometry (DXA); Height (Z-scores for standing height/lying-length-for-age); Markers of Bone Metabolism: Measurements of Serum osteocalcin (OCN) and bone-specific alkaline phosphatase (BSAP), carboxyterminal telopeptide of type I collagen (ICTP) and carboxyterminal telopeptide al chain of type I collagen (CTX); Flexibility and Muscle Strength: Biodex and Physical Therapy evaluations, including 6 minute walk study (The Biodex III isokinetic strength testing system is used to assess strength at the knee and elbow for each participant); Active Joint Range of Motion (ROM); Child Health Assessment Questionnaire/Health Assessment Questionnaire (CHAQ/HAQ) Disability Index Score; Electromyographic (EMG) and/or Oxygen Utilization to Monitor an individual's cardiorespiratory fitness: peak oxygen uptake (VO2 peak) during exercise testing; Apnea/Hypopnea Index (AHI); Forced Vital Capacity (FVC); Left Ventricular Mass (LVM).

In certain embodiments, a method of diagnosing and/or treating MPSI in a patient, or monitoring treatment, is provided. The method involves obtaining a cerebrospinal fluid or plasma sample from a human patient suspected of having MPSI; detecting spermine concentration levels in the sample; diagnosing the patient with a mucopolysaccharidosis selected from MPS I in the patient having spermine concentrations in excess of 1 ng/mL; and delivering an effective amount of human alpha-L-iduronidase (hIDUA) to the diagnosed patient as provided herein, e.g., using a device as described herein.

In another aspect, the method involves monitoring and adjusting MPSI therapy. Such method involves obtaining a cerebrospinal fluid or plasma sample from a human patient undergoing therapy for MPSI; detecting spermine concentration levels in the sample by performing a mass spectral analysis; adjusting dosing levels of the MPSI therapeutic. For example, “normal” human spermine concentrations are about 1 ng/mL or less in cerebrospinal fluid. However, patients having untreated MPSI may have spermine concentration levels of greater than 2 ng/mL and up to about 100 ng/mL. If a patient has levels approaching normal levels, dosing of any companion ERT may be lowered. Conversely, if a patient has higher than desired spermine levels, higher doses, or an additional therapy, e.g., ERT may be provided to the patient.

Spermine concentration may be determined using a suitable assay. For example the assay described in J Sanchez-Lopez, et al, “Underivatives polyamine analysis is plant samples by ion pair liquid chromatography coupled with electrospray tandem mass spectrometry,” Plant Physiology and Biochemistry, 47 (2009): 592-598, avail online 28 Feb. 2009; M R Hakkinen et al, “Analysis of underivatized polyamines by reversed phase liquid chromatography with electrospray tandem mass spectrometry”, J Pharm Biomec Analysis, 44 (2007): 625-634, quantitative isotope dilution liquid chromatography (LC)/mass spectrometry (MS) assay. Other suitable assays may be used.

In some embodiments, efficacy of a therapeutic described herein is determined by assessing neurocognition at week 52 post-dose in pediatric subjects with MPS I who have an early-stage neurocognitive deficit. In some embodiments, efficacy of a therapeutic described herein is determined by assessing the relationship of CSF glycosaminoglycans (GAG) to neurocognition in an MPS I patient. In some embodiments, efficacy of a therapeutic described herein is determined by evaluating the effect of the therapeutic on physical changes to the CNS in an MPS I patient as measured by magnetic resonance imaging (MRI), e.g., volumetric analysis of gray and white matter and CSF ventricles. In some embodiments, efficacy of a therapeutic described herein is determined by evaluating the pharmacodynamic effect of the therapeutic on biomarkers, (e.g., GAG, HS) in cerebrospinal fluid (CSF), serum, and urine of an MPS I patient. In some embodiments, efficacy of a therapeutic described herein is determined by evaluating the impact of the therapeutic on quality of life (QOL) of an MPS I patient. In some embodiments, efficacy of a therapeutic described herein is determined by evaluating the impact of the therapeutic on motor function of an MPS I patient. In some embodiments, efficacy of a therapeutic described herein is determined by evaluating the effect of the therapeutic on growth and on developmental milestones of an MPS I patient.

As expressed from the rAAV vector described herein, expression levels of at least about 2% as detected in the CSF, serum, or other tissue, may provide therapeutic effect. However, higher expression levels may be achieved. Such expression levels may be from 2% to about 100% of normal functional human IDUA levels. In certain embodiments, higher than normal expression levels may be detected in CSF, serum, or other tissue.

In certain embodiments, the methods of treating, preventing, and/or ameliorating MPS I and/or symptoms thereof described herein result in a significant increase in intelligence quotient (IQ) in treated patients, as assessed using Bayley's Infantile Development Scale for Hurler subjects. In certain embodiments, the methods of treating, preventing, and/or ameliorating MPS I and/or symptoms thereof described herein result in a significant increase in neurocognitive IQ in treated patients, as measured by Wechsler Abbreviated Scale of Intelligence (WASI) for Hurler-Scheie subjects. In certain embodiments, the methods of treating, preventing, and/or ameliorating MPS I and/or symptoms thereof described herein result in a significant increase in neurocognitive DQ in treated patients, as assessed using Bayley Scales of Infant Development.

In certain embodiments, the methods of treating, preventing, and/or ameliorating MPS I and/or symptoms thereof described herein result in a significant increase in functional human IDUA levels. In certain embodiments, the methods of treating, preventing, and/or ameliorating MPS I and/or symptoms thereof described herein result in a significant decrease in GAG levels, as measured in a sample of a patient's serum, urine and/or cerebrospinal fluid (CSF).

### Combination Therapies

Combinations of gene therapy delivery of the rAAV.hIDUA to the CNS accompanied by systemic delivery of hIDUA are encompassed by the methods of the invention. Systemic delivery can be accomplished using ERT (e.g., using Aldurazyme®), or additional gene therapy using an rAAV.hIDUA.

In certain embodiments, an intrathecal administration of rAAV.hIDUA is be co-administered with a second AAV.hIDUA injection, e.g., directed to the liver. In such an instance, the vectors may be same. For example, the vectors may have the same capsid and/or the same vector genomic sequences. Alternatively, the vector may be different. For example, each of the vector stocks may designed with different regulatory sequences (e.g., each with a different tissue-specific promoter), e.g., a liver-specific promoter and a CNS-specific promoter. Additionally, or alternatively, each of the vector stocks may have different capsids. For example, a vector stock to be directed to the liver may have a capsid selected from AAV8, AAVhu68, AAV9, AAVrh91, AAVrh64R1, AAVrh64R2, AAVrh8, AAVrh10, AAV3B, or AAVdj, among others. In such a regimen, the doses of each vector stock may be adjusted so that the total vector delivered intrathecally is within the range of about 1×10⁸ GC to ×1×10¹⁴ GC; in other embodiments, the combined vector delivered by both routes is in the range of 1×10¹¹ to 1×10¹⁶. Alternatively, each vector may be delivered in an amount of about 10⁸ GC to about 10¹² GC/vector. Such doses may be delivered substantially simultaneously, or at different times, e.g., from about 1 day to about 12 weeks apart, or about 3 days to about 30 days, or other suitable times.

In some embodiments, the patient is co-administered an rAAV.hIDUA via liver-directed and intrathecal injections. In some embodiments a method for treatment comprises: (a) dosing a patient having MPS I and/or the symptoms associated with Hurler, Hurler-Scheie and Scheie syndromes with a sufficient amount of hIDUA enzyme or liver directed rAAV-hIDUA to induce transgene-specific tolerance; and (b) administering an rAAV.hIDUA to the patient's CNS, which rAAV.hIDUA directs expression of therapeutic levels of hIDUA in the patient.

In a further embodiment, a method of treating a human patient having MPSI and/or the symptoms associated with Hurler, Hurler-Scheie and Scheie syndromes is provided which involves tolerizing a patient having MPSI and/or the symptoms associated with Hurler, Hurler-Scheie and Scheie syndromes with a sufficient amount of hIDUA enzyme or liver-directed rAAV-hIDUA to induce transgene-specific tolerance, followed by CNS-directed rAAV-mediated delivery of hIDUA to the patient. In certain embodiments, the patient is administered an rAAV.hIDUA via liver-directed injections e.g., when the patient is less than 4 weeks old (neonatal stage) or an infant in order to tolerize the patient to hIDUA, and the patient is subsequently administered rAAV.hIDUA via intrathecal injections when the patient is an infant, child, and/or adult to express therapeutic concentrations of hIDUA in the CNS.

In one example, the MPSI patient is tolerized by delivering hIDUA to the patient within about two weeks of birth, e.g., within about 0 to about 14 days, or about 1 day to 12 days, or about day 3 to about day 10, or about day 5 to about day 8, i.e., the patient is a newborn infant. In other embodiments, older infants may be selected. The tolerizing dose of hIDUA may be delivered via rAAV. However, in another embodiment, the dose is delivered by direct delivery of the enzyme (enzyme replacement therapy). Methods of producing recombinant hIDUA in Chinese hamster ovary (CHO) cells and soluble rhIDUA in tobacco cells [L H Fu, et al, Plant Science (Impact Factor: 3.61). December 2009; 177(6):668-675] or plant seeds [X He et al, Plant Biotechnol J. 2013 December; 11(9): 1034-1043] have been described in the literature.

Additionally, a recombinant hIDUA is commercially produced as Aldurazyme® (laronidase); a fusion protein of an anti-human insulin receptor monoclonal antibody and alpha-L-iduronidase [AGT-181; ArmaGen, Inc] may be useful. Although currently less preferred, the enzyme may be delivered via “naked” DNA, RNA, or another suitable vector. In one embodiment, the enzyme is delivered to the patient intravenously and/or intrathecally. In another embodiment, another route of administration is used (e.g., intramuscular, subcutaneous, etc). In one embodiment, the MPSI patient selected for tolerizing is incapable of expressing any detectable amounts of hIDUA prior to initiation of the tolerizing dose. When recombinant human IDUA enzyme is delivered, intrathecal rhIDUA injections may consist of about 0.58 mg/kg body weight or about 0.25 mg to about 2 mg total rhIDUA per injection (e.g., intravenous or intrathecal). For example, 3 cc of enzyme (e.g., approximately 1.74 mg Aldurazyme® (laronidase)) diluted with 6 cc of Elliotts B® solution for a total injection of 9 cc. Alternatively, a higher or lower dose is selected. Similarly, when expressed from a vector, lower expressed protein levels may be delivered. In one embodiment, the amount of hIDUA delivered for tolerizing is lower than a therapeutically effective amount. However, other doses may be selected.

Typically, following administration of the tolerizing dose, the therapeutic dose is delivered to the subject, e.g., within about three days to about 6 months post-tolerizing dose, more preferably, about 7 days to about 1 month post-tolerizing dose. However, other time points within these ranges may be selected, as may longer or shorter waiting periods.

As an alternative, immunosuppressive therapy may be given in addition to the vector—before, during and/or subsequent to vector administration. Immunosuppressive therapy can include prednisolone, mycophenolate mofetil (MMF) and tacrolimus or sirolimus as described supra. A tacrolimus-free regimen described infra may be preferred.”

### Kits

In certain embodiments, a kit is provided which includes a concentrated expression cassette (e.g., in a viral or non-viral vector) suspended in a formulation (optionally frozen), optional dilution buffer, and devices and components required for intrathecal, intracerebroventricular or intracisternal administration. In another embodiment, the kit may additional or alternatively include components for intravenous delivery. In one embodiment, the kit provides sufficient buffer to allow for injection. Such buffer may allow for about a 1:1 to a 1:5 dilution of the concentrated vector, or more. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

It should be understood that the compositions in the kits described herein are intended to be applied to other compositions, regiments, aspects, embodiments and methods described across the Specification.

### Devices

In one aspect, the compositions provided herein may be administered intrathecally via the method and/or the device described, e.g., in WO 2017/136500, which is incorporated herein by reference in its entirety. In summary, the method comprises the steps of advancing a spinal needle into the cisterna magna of a patient, connecting a length of flexible tubing to a proximal hub of the spinal needle and an output port of a valve to a proximal end of the flexible tubing, and after said advancing and connecting steps and after permitting the tubing to be self-primed with the patient's cerebrospinal fluid, connecting a first vessel containing an amount of isotonic solution to a flush inlet port of the valve and thereafter connecting a second vessel containing an amount of a pharmaceutical composition to a vector inlet port of the valve. After connecting the first and second vessels to the valve, a path for fluid flow is opened between the vector inlet port and the outlet port of the valve and the pharmaceutical composition is injected into the patient through the spinal needle, and after injecting the pharmaceutical composition, a path for fluid flow is opened through the flush inlet port and the outlet port of the valve and the isotonic solution is injected into the spinal needle to flush the pharmaceutical composition into the patient. This method and this device may each optionally be used for intrathecal delivery of the compositions provided herein. Alternatively, other methods and devices may be used for such intrathecal delivery.

It should be understood that the compositions in the device described herein are intended to be applied to other compositions, regiments, aspects, embodiments and methods described across the Specification.

### EXAMPLES

The invention is now described with reference to the following examples. These examples are provided for the purpose of illustration only and the invention should in no way be construed as being limited to these examples but rather should be construed to encompass any and all variations that become evident as a result of the teaching provided herein.

### Example 1: Materials and Methods

### Animals

All animal procedures were approved by the Institutional Animal Care and Use Committee of the University of Pennsylvania. Rhesus macaques (Macaca mulatta) were procured from Covance Research Products, Inc. and Primgen/Prelabs Primates. Animals were housed in an Association for Assessment and Accreditation of Laboratory Animal Care (AAALAC) International-accredited Nonhuman Primate Research Program facility at the University of Pennsylvania in stainless steel squeeze-back cages. Animals received varied enrichments such as food treats, visual and auditory stimuli, manipulatives, and social interactions. C56BL/6J mice (stock #000664) were purchased from the Jackson Laboratory. Animals were housed in an AAALAC International-accredited mouse barrier vivarium at the Gene Therapy Program, University of Pennsylvania, in standard caging of 2 to 5 animals per cage with enrichment (Nestlets nesting material). Cages, water bottles, and bedding substrates were autoclaved in the barrier facility and cages were changed once per week. An automatically controlled 12-hour light/dark cycle was maintained. Each dark period began at 1900 hours (±30 minutes). Irradiated laboratory rodent food was provided ad libitum.

Animals were visually monitored daily by the animal care and/or veterinary staff for any conditions requiring possible intervention. This included monitoring the general appearance of the animal for signs of toxicity, distress, and/or changes in behavior. On select study time points, animals were also monitored for additional parameters including, but not limited to, vital signs and had blood collected for clinical pathology. All animals enrolled in the described study had a neurological assessment up to once per month. The neurological examination involved cage-side evaluation of mentation, posture, proprioception, and gait, as well as a restrained evaluation of cranial nerves, motor strength, and reflexes. Animals were observed daily by care staff for any signs of pain or discomfort such as changes in behavior or significant changes in appetite. Any clinical abnormalities were reported to study veterinarians and the study director, none of which were suspected to be related to test article administration.

### Vectors

The AAV9.PHP.B trans-plasmid (pAAV2/PHP.B) was generated with a QuikChange Lightning Site-Directed Mutagenesis Kit (Agilent Technologies, Cat #210515) using pAAV2/9 as the template according to the manufacturer's instructions. AAV vectors were produced and titrated as described previously (37). Briefly, HEK293 cells were triple-transfected and the culture supernatant was harvested, concentrated, and purified with an iodixanol gradient. The purified vectors were titrated with droplet digital PCR using primers targeting the rabbit beta-globin polyA sequence as previously described (38). Engineered sequences encoding human alpha-L-iduronidase (hIDUA) were cloned under the CB7 promoter. MicroRNA sequences were obtained on the public database mirbase.org (Hsa-mir-183 MI0000273; Hsa-mir-182 MI0000272; Hsa-mir-96 MI0000098; Hsa-mir-145 MI0000461). Four tandem repeats of the target for the DRG-enriched miR were cloned in the 3′ untranslated region (UTR) of green fluorescent protein (GFP) or hIDUA cis-plasmids.

### In Vivo Studies and Histology

### Mice

Mice received 1×10¹² genome copies (GCs; 5×10¹³ GC/kg) of AAV-PHP.B, or 4×10¹² GCs (2×10¹⁴ GC/kg) of AAV9 vectors encoding enhanced GFP with or without DRG-miR targets in 0.1 mL PBS (vehicle) via the lateral tail vein and were euthanized by inhalation of CO₂ 21 days post-injection. Tissues were promptly collected, starting with brain, and immersion-fixed in 10% neutral buffered formalin for about 24 h, washed briefly in phosphate-buffered saline (PBS), and equilibrated sequentially in 15% and 30% sucrose in PBS at 4° C. Tissues were then frozen in optimum cutting temperature embedding medium and cryosectioned for direct GFP visualization (brain was sectioned at 30 μm, and other tissues at 8 μm thickness). Images were acquired with a Nikon Eclipse Ti-E fluorescence microscope. GFP expression in DRGs was analyzed by immunohistochemistry (IHC). Spinal columns with DRGs were fixed in formalin for 24 h, decalcified in 10% ethylenediaminetetraacetic acid (pH 7.5) until soft, and paraffin-embedded following standard protocols. Sections were deparaffinized through an ethanol and xylene series, boiled for 6 min in 10 mM citrate buffer (pH 6.0) to perform antigen retrieval, blocked sequentially with 2% H₂O₂ (15 min), avidin/biotin blocking reagents (15 min each; Vector Laboratories), and blocking buffer (1% donkey serum in PBS+0.2% Triton for 10 min) followed by incubation with primary (1 h at 37° C.) and biotinylated secondary antibodies (diluted 1:500, 45 min; Jackson ImmunoResearch diluted in blocking buffer. A rabbit antibody against GFP was used as the primary antibody (NB600-308, Novus Biologicals; diluted 1:500). A Vectastain Elite ABC kit (Vector Laboratories) with DAB as substrate enabled visualization of bound antibodies as brown precipitate.

### Non-Human Primates (NHP)

NHP received 3.5×10¹³ GCs of AAVhu68.GFP vectors or 1×10¹³ GCs of AAVhu68.hIDUA vectors in a total volume of 1 mL of sterile artificial CSF (vehicle) injected into the cisterna magna, under fluoroscopic guidance as previously described (40). Period blood collection and cerebrospinal fluid (CSF) taps were performed for safety readouts. Serum chemistry, hematology, coagulation, and CSF analyses were performed by the contract facility Antech Diagnostics. Animals were euthanized with intravenous pentobarbital overdose and necropsied; the tissues were then harvested for comprehensive histopathologic examination. Collected tissues were immediately fixed in formalin and paraffin embedded. For histopathology, tissue sections were stained with hematoxylin and eosin following standard protocols. IHC for GFP expression was carried out as described for the mouse studies but using a different antibody against GFP (goat antibody NB100-1770, Novus Biologicals; diluted 1:500, incubated overnight at 4° C.). Immunostaining for hIDUA was performed using a sheep antibody against hIDUA (AF4119, R&D Systems; diluted 1:200) following the above protocol for IHC. In addition, sections were stained for hIDUA by immunofluorescence (IF) using the same primary antibody. For IF, sections were deparaffinized and treated for antigen retrieval as described above, and then blocked with 1% donkey serum in PBS+0.2% Triton for 25 min followed by sequential incubation with primary (2 h at room temperature, diluted 1:50) and FITC-labeled secondary (45 min; Jackson ImmunoResearch; diluted 1:100) antibodies diluted in blocking buffer. Sections were mounted in Fluoromount G with DAPI as a nuclear counterstain.

In situ hybridization (ISH) was performed using probes specific for the RNA transcribed from the vector genome that do not bind to endogenous monkey IDUA RNA. Z-shaped probe pairs were synthesized by Life Technologies and ISH was performed on paraffin sections using the Life Technologies ViewRNA ISH Tissue Assay kit according to the manufacturer's protocol. The deposition of Fast Red precipitates indicating positive signals was imaged by fluorescence microscopy using a rhodamine filter set. Tissue sections with IDUA IHC were scanned for quantification purposes using an Aperio Versa slide scanner (Leica Biosystems).

### Histopathology and Morphometry

### Pathology Scoring

A board-certified Veterinary Pathologist who was blinded to the vector group established severity grades defined with 0 as absence of lesion, 1 as minimal (<10%), 2 mild (10-25%), 3 moderate (25-50%), 4 marked (50-95%), and 5 severe (>95%). Dorsal axonopathy scores were established in each animal from at least 3 cervical, 3 thoracic, and 3 lumbar sections; the DRG severity grades were established from at least 3 cervical, 3 thoracic, and 3 lumbar segments; and the median nerve score was the sum of axonopathy and fibrosis severity grades with a maximal possible score of 10 and was established on the distal and proximal portions of left and right nerves. For quantification of transgene expression, a board-certified Veterinary Pathologist counted cells immunostained with anti-GFP or anti-hIDUA antibodies by comparing to the signal from control slides obtained from untreated animals. The total number of positive cells per ×20 magnification field was counted manually using the ImageJ or Aperio Image Scope cell counter tool on a minimum of five fields per structure and per animal.

### ISH Quantification

The cytoplasmic ISH signal of DRG neurons that showed a nuclear signal (contained vector genomes) within a given section was quantified. Stained slides were scanned and screenshots were taken to cover the whole area of the DRGs to be analyzed. Using the Fiji version of ImageJ, images showing only the ISH channel were thresholded at identical setting and synchronized (using the Window Synchronization tool) with a corresponding image showing the ISH and DAPI channels. The percentage of area occupied by the ISH signal in the cytoplasmic area shown in the thresholded image was then determined with the ‘Measure’ tool.

### Vector Biodistribution

NHP tissue DNA was extracted with a QIAamp DNA Mini Kit (Qiagen Cat #51306) and vector genomes were quantified by qRT-PCR using Taqman reagents (Applied Biosystems, Life Technologies) and primers/probes targeting the rBG polyadenylation sequence of the vectors.

Immunology Peripheral blood T-cell responses against hIDUA were measured by interferon gamma enzyme-linked immunosorbent spot assays according to previously published methods and used peptide libraries specific for the hIDUA transgene. Positive response criteria were >55 spot forming units per 10⁶ lymphocytes and three times the medium negative control upon no stimulation. In addition, T-cell responses were assayed in lymphocytes extracted from spleen, liver, and deep cervical lymph nodes after necropsy on study day 90. Antibodies to hIDUA were measured in serum (1:1,000 sample dilution).

Cytokine/Chemokine analysis: CSF samples were collected and stored at −80° C. until the time of analysis. CSF samples were analyzed using a Milliplex MAP kit containing the following analytes: sCD137, Eotaxin, sFasL, FGF-2, Fractalkine, Granzyme A, Granzyme B, IL-la, IL-2, IL-4, IL-6, IL-16, IL-17A, IL-17E/IL-25, IL-21, IL-22, IL-23, IL-28A, IL-31, IL-33, IP-10, MIP-3a, Perforin, TNFβ. CSF samples were evaluated in duplicate and analyzed in a FLEXMAP 3D instrument using Luminex xPONENT 4.2; Bio-Plex Manager Software 6.1. Only samples with a % CV of less than 20% were included in the analysis.

### In Vitro Studies

miR183 Expression

MIR183 human microRNA expression plasmid was modified from Origene MI0000273 vector by deleting the KpnI-PstI fragment encoding GFP and partial internal ribosome entry sites. We confirmed the lack of GFP expression from the modified vector by transient transfection and anti-GFP immunoblotting. Polyethylenimine-mediated transient transfection was performed in HEK293 cells with GFP cis-plasmids harboring microRNA binding sites located in the 3′-UTR of the GFP expression cassette. At 72 hours post-transfection, cells were lysed in 50 mM Tris-HCl, pH 8.0, 150 mM NaCl, and 0.5% Triton X-100 with protease inhibitors. A total of 13 μg of cell lysates was used for anti-GFP immunoblotting followed by electrochemiluminescence-based signal detection and quantification. Experiments were performed in triplicate for statistical analysis.

miR183 quantification (RT-PCR)

Human DRG and spinal cord tissues were sourced from Anabios, Inc. Lumbar DRG and spinal cord were originally obtained from a 25-year-old, male Caucasian (a consented organ donor with no history of neuropathic pain) and stored immediately in RNALater (Ambion). NHP Rhesus monkey tissues from three animals were obtained from previous studies and stored in a −80° C. freezer. A miRNeasy Mini Kit was used for total RNA isolation (Qiagen) and the extracted RNAs were reverse transcribed with a TaqMan MicroRNA Reverse Transcription Kit (Applied Biosystems), according to the protocol instructions. qRT-PCR was performed to determine the abundance of miR183 in different tissues, using the TaqMan MicroRNA Assay kit with primers specific to hsa-miR-183-5p (Assay ID 002269) and RNU6B (Assay ID 00193) (Applied Biosystems Inc.) according to the manufacturer's instructions. Each qRT-PCR assay was conducted in triplicate using cDNA derived from 100 ng total RNA and analyzed by the comparative threshold cycle (Ct) method. The average expression miR183 was normalized with RNU6B as an endogenous control gene, using the 2^{-ΔΔCt} method.

### Statistical Analysis

Statistical differences between the control group (no miR target vector) and the test group (miR targets vectors) were assessed using the non-parametric two-sided Wilcoxon rank-sum test, alpha level of 0.05 (R version 4.0.0) with the exception of cytokine analysis performed using the non-parametric Kruskal-Wallis test for each cytokine and time point to test for difference between groups (alpha=0.05; R version 4.0.0). For GFP expression in mice, statistical differences between groups were assessed using a parametric one-way ANOVA followed by Tukey's multiple comparison test, alpha level of 0.05. Data set passed the Shapiro-Wilk normality test (GraphPad Prism version 7.05).

### Example 2: MicroRNA-Mediated Inhibition of Transgene Expression Reduces Dorsal Root Ganglion Toxicity by AAV Vectors

Delivering adeno-associated virus (AAV) vectors into the central nervous system (CNS) of non-human primates (NHPs) via the blood or cerebral spinal fluid is associated with dorsal root ganglion (DRG) toxicity. Conventional immune-suppression regimens does not prevent this toxicity, possibly because it may be caused by high transduction rates, which can, in turn, cause cellular stress due to an overabundance of the transgene product in target cells. We developed an approach to eliminate DRG toxicity by introducing sequence targets for miR183 into the vector genome within the 3′ untranslated region of the corresponding transgene mRNA.

### AAV Vectors Cause DRG Degeneration in NHPs

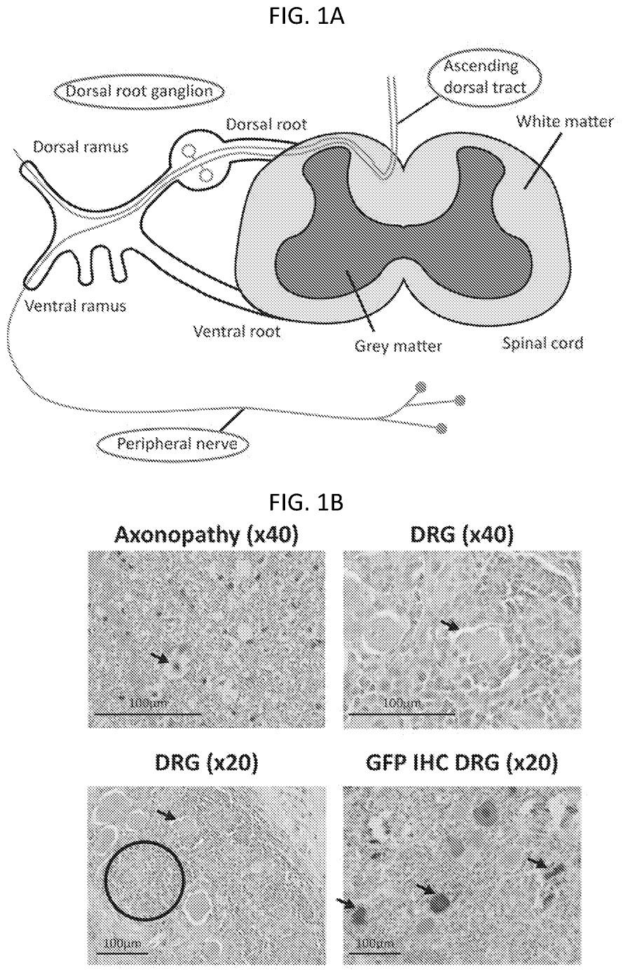

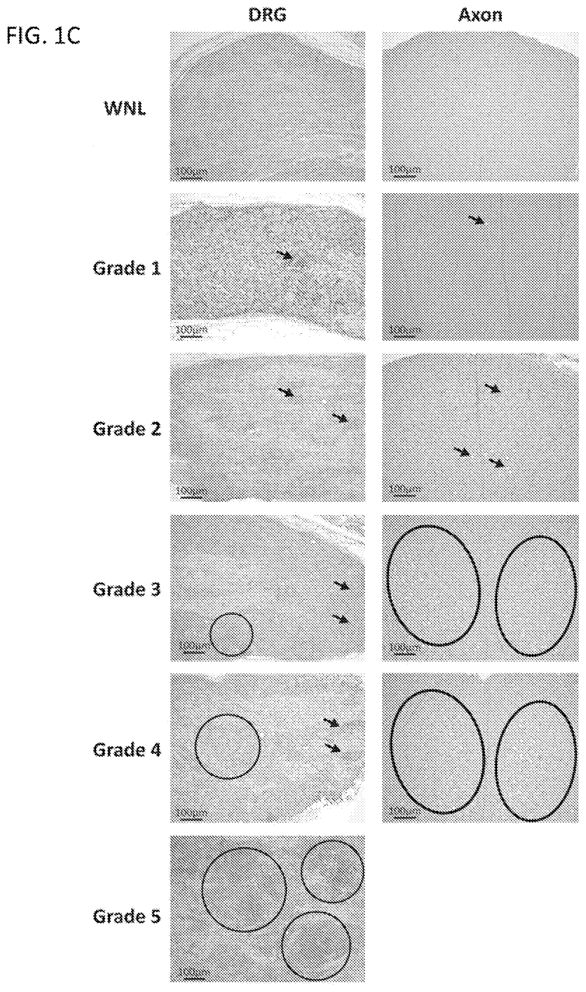

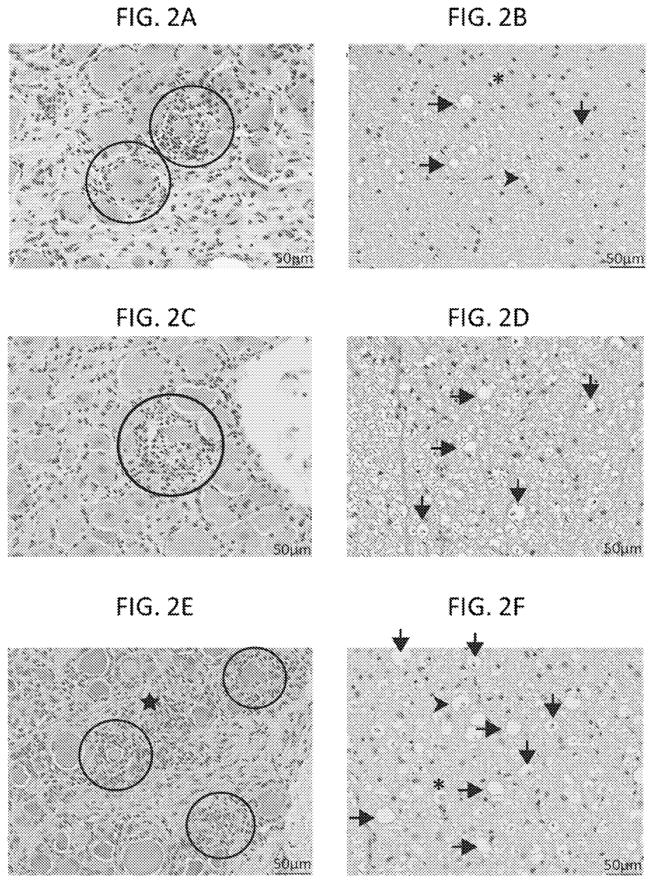

Based on our experience with DRG toxicity in NHPs, we developed a system to quantify the severity of toxicity. We evaluate cell bodies located along the spinal cord in the DRGs, the axons within the peripheral nerves, and the axons that ascend the dorsal white-matter tracts (FIG. 1A). We believe the primary lesion is degeneration of the sensory neuron cell body located in the DRG. Microscopic evaluation highlights a range of DRG lesions. Early neuronal degeneration consists of otherwise normal neuronal cell bodies encircled by proliferating satellite and microglial and infiltrated mononuclear cells (FIG. 2A). Later stages of neuronal degeneration and neuronophagia comprise small, irregular, or angular neuronal cell bodies with diffuse cytoplasmic hypereosinophilia and loss of nuclei (FIG. 1B; FIG. 2C and FIG. 2E). Cells highly expressing transgene protein are more likely to undergo degeneration as evidenced by transgene product immunostaining in animals that received an ICM administration of an AAV vector expressing green fluorescent protein (GFP; FIG. 1B). Secondary to the cell body death is axonopathy (degeneration of the distal and proximal axons). The dorsal white matter tracts of the spinal cord exhibit dilated myelin sheaths, with and without myelomacrophages and axonal debris, along with swollen axons, consistent with axonal degeneration (FIG. 1B; FIG. 2B, FIG. 2D, and FIG. 2F). FIG. 1C illustrates examples of varying DRG toxicity and spinal cord axonopathy severity. The grades are based on the proportion of affected tissue at high-power field histopathologic examination: 1 minimal (<10%), 2 mild (10-25%), 3 moderate (25-50%), 4 marked (50-95%) and 5 severe (>95%).

Our total experience of adolescent/adult NHPs administered AAV vectors into the CSF via ICM or lumbar puncture (LP) route totals 219 monkeys spanning 27 studies that encompass previous published toxicology studies and the two NHP experiments described below, as well as a number of unpublished studies. This experience includes five capsids, 20 transgenes, five promoters (CAG, CB7, UBC, hSyn, and MeP426), doses from 1×10¹² GC to 3×10¹⁴ GC, vector purified by gradients or columns, three formulations (phosphate-buffered saline and two different artificial CSFs), and rhesus and cynomolgus macaques at various developmental stages. In every experimental group, we observed DRG toxicity and axonopathy. The pathology peaks about one month after injection and does not progress for up to six months, which is the longest period evaluated in mature macaques. In most cases, the pathology is mild to moderate and NHPs do not present clinical signs suggestive of neuropathic pain. However, high doses of vectors expressing GFP injected ICM can lead to severe pathology associated with ataxia.

miRNAs Specifically Expressed in DRG Neurons can Ablate AAV Transgene Expression

We evaluated several mechanisms when considering ways to mitigate DRG toxicity. In previous studies, we analyzed the role of destructive adaptive immune responses to the transduced DRGs by immune-suppressing NHPs that were ICM-administered AAV9 vectors expressing human alpha-L-iduronidase (hIDUA) or human iduronate 2-sulfatase. Treatment with mycophenolate mofetil (MMF) and rapamycin blunted the adaptive immune response to the vector and transgene product but did not impact DRG toxicity or axonopathy.

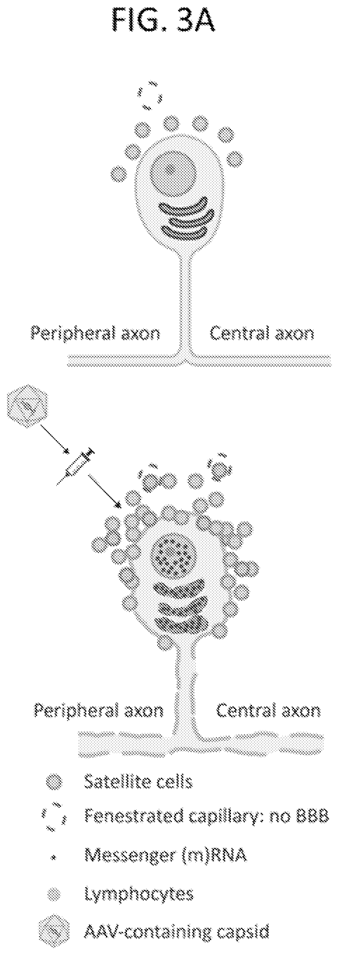

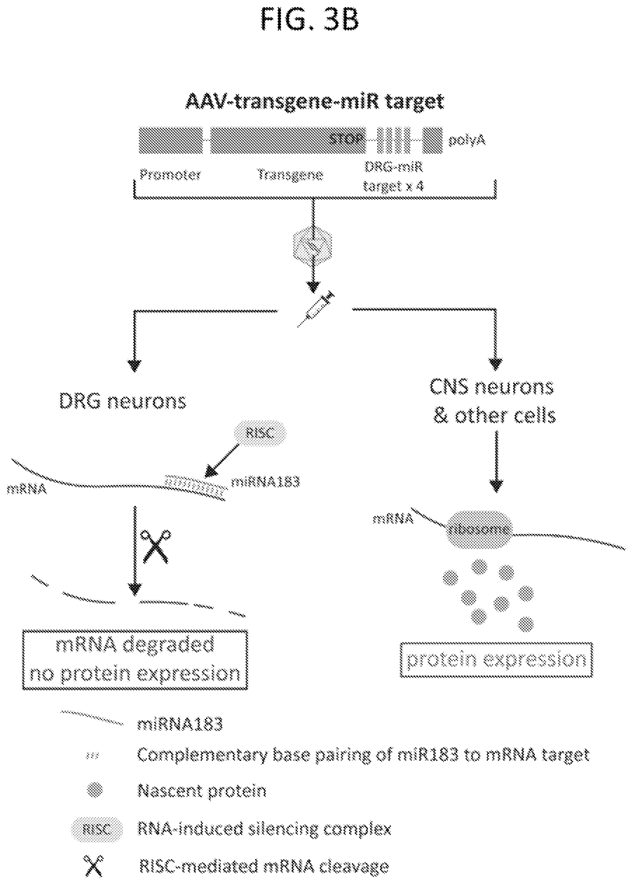

Our operating model contends that overexpression of the transgene product in highly transduced DRGs leads to neuronal injury and degeneration of the cell body and associated axons followed by a reactive inflammatory response (FIG. 3A). To test this hypothesis, we designed a strategy for specifically ablating transgene expression. We used an approach previously deployed for restricting lentiviral vector expression in hematopoietic-derived cells or de-targeting the liver, heart, or muscle after AAV-mediated gene transfer. The approach involved cloning miRNA targets that are solely expressed in DRG neurons into the 3′ untranslated regions of the transgene (FIG. 3B). Any mRNA expressed from the vector would be destroyed by the endogenously expressed miRNA.

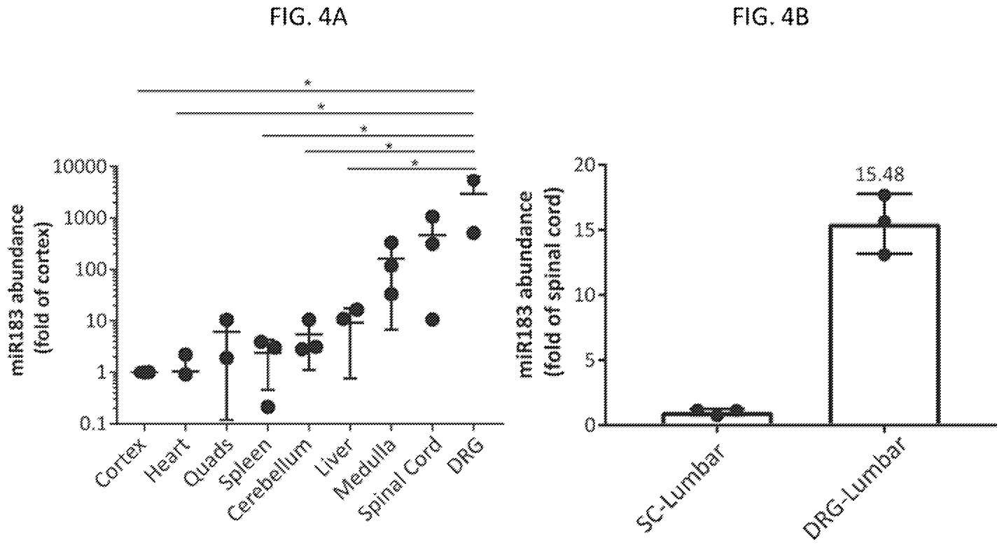

Screening existing miRNA databases and literature revealed that the miRNA183 cluster was a good candidate for this strategy (miRBase Tracker for miR183: MI0000273). This cluster contains three miRNAs (96, 182, and 183), all expressed from a polycistronic pri-miRNA. Under normal conditions, expression of this complex is largely restricted to neurons of the olfactory epithelium, ear, retina, and DRG as demonstrated in zebrafish, mouse, rat, and human tissues. We verified this sensory neuron-specific expression pattern from three NHPs and one human donor by quantitative real-time PCR (qRT-PCR). We showed that miR183 is at least ten times more abundant in NHP and human DRG compared to the spinal cord, and that in NHPs the lowest abundance is in the cerebral cortex (1,000 fold lower than in DRG) followed by the heart, spleen, skeletal muscle, cerebellum, liver, and medulla (FIG. 4A and FIG. 4B). Outside of sensory neurons, the cluster can be upregulated in several pathologic states, including cancer and autoimmune diseases. Included in our initial screen was the less-well characterized miRNA145 expressed in rat DRGs. Target sequences for the miRNAs within all of these complexes are conserved between mice, monkeys, and humans (see miRBase trackers for miR183: MI0000273, MI0003084, MI0000225; miR182: MI0000272, MI0000224, MI0002815; miR96: MI0000098, MI0000583, MI0003085; and miR145: MI0000461, MI0000169, MI0002558).

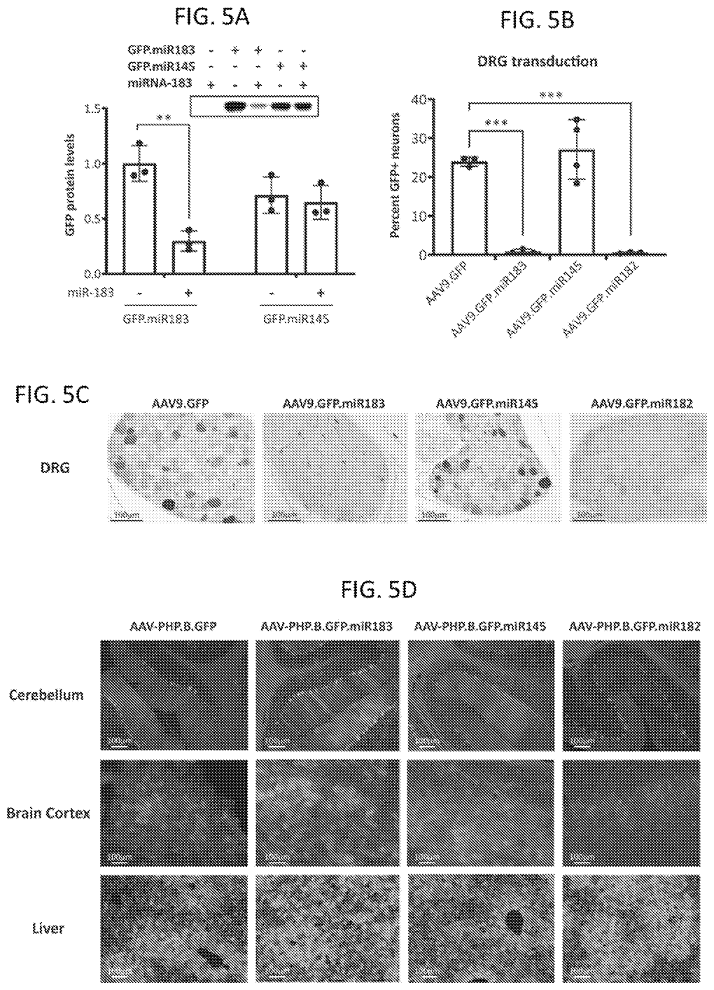

We used an in vitro assay to evaluate the activity and specificity of the miRNA strategy. We constructed AAV cis-plasmids to include four repeat concatemers of the target miRNA sequences in the 3′ untranslated region of the expression cassette. AAV cis-plasmids were co-transfected with plasmids expressing miR183. Expression of the transgene GFP was reduced in the presence of miR183 only when it contained the cognate recognition sequence (FIG. 5A; p=0.0027).

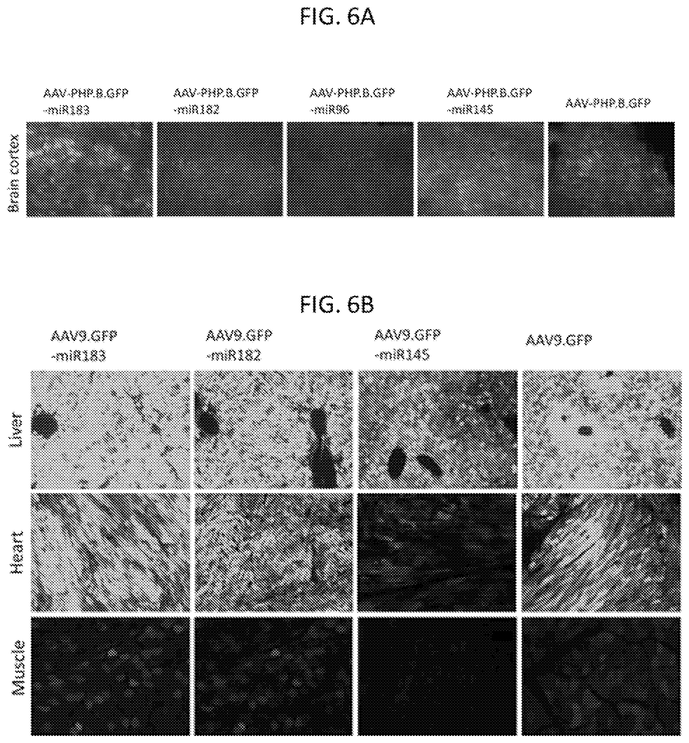

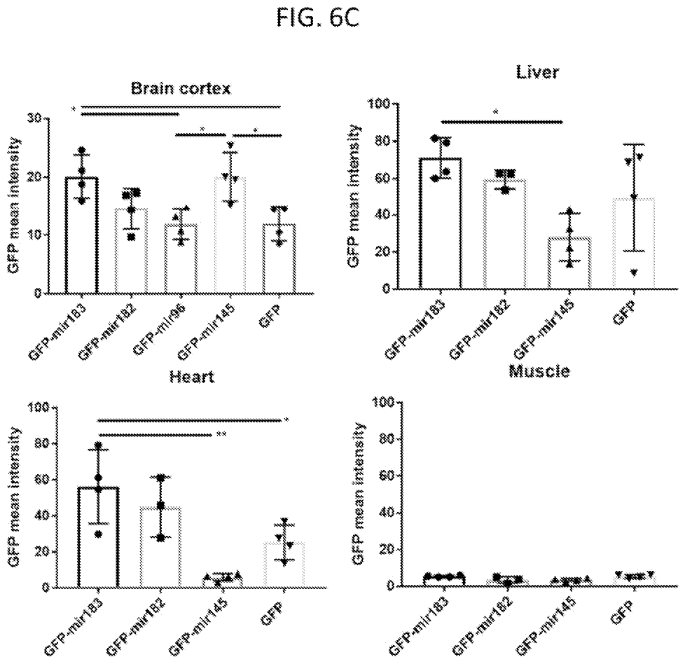

The in vivo activity and specificity of potential miRNA targets within AAV vectors were screened in C57B1/6J mice. We evaluated GFP-expressing vectors with or without miRNA targets from two members of the miRNA183 complex (miR182 and miR183) as well as miR145. We initially tested miR96, another member of the 183 complex, but eliminated it due to decreased GFP-miR96 transgene expression in mice cortices compared to miR183 (p=0.03) and miR145 (p=0.03) (FIG. 6A and FIG. 6C). Animals received high-dose intravenous (IV) injections of AAV9 to target DRGs or high-dose AAV-PHP.B injections to target the CNS. Animals were necropsied on day 21 and analyzed for GFP expression in DRGs by immunohistochemistry (IHC) and direct-fluorescence microscopy in brain and liver (AAV-PHP.B) or liver, heart and muscles (AAV9). Expression of GFP in DRG neurons was substantially reduced with vectors containing miR183 targets (p=0.00007) and miR182 targets (p=0.00003) but not miR145 targets (FIG. 5B and FIG. 5C). There was no reduction of expression in liver, heart, muscle, or brain cortex with vectors containing the miR183 targets and expression was enhanced when compared to control GFP vectors in brain cortex (p=0.03), and heart (p=0.04) (FIG. 5D, FIG. 6A-FIG. 6C). In this mouse experiment, we were unable to assess the impact of miR183 target-mediated transgene suppression on pathology since vector-induced DRG toxicity has only been observed in NHPs. The reason for this species difference is unknown.

A vector genome for ITR.CB7.CI.eGFP.miR145(four copies).rBG.ITR is provided in SEQ ID NO: 10, a vector genome for ITR.CB7.CI.GFP.miR182(four copies).rBG.ITR is provided in SEQ ID NO: 11, a vector genome for ITR.CB7.CI.GFP.miRNA96(four copies).rBG.ITR is provided in SEQ ID NO: 12, and a vector genome for ITR.CB7.CI.GFP.miR183(four copies).rBG.ITR is provided in SEQ ID NO: 13.

Restricted Transgene Expression by miR183 Reduces DRG Toxicity in NHPs

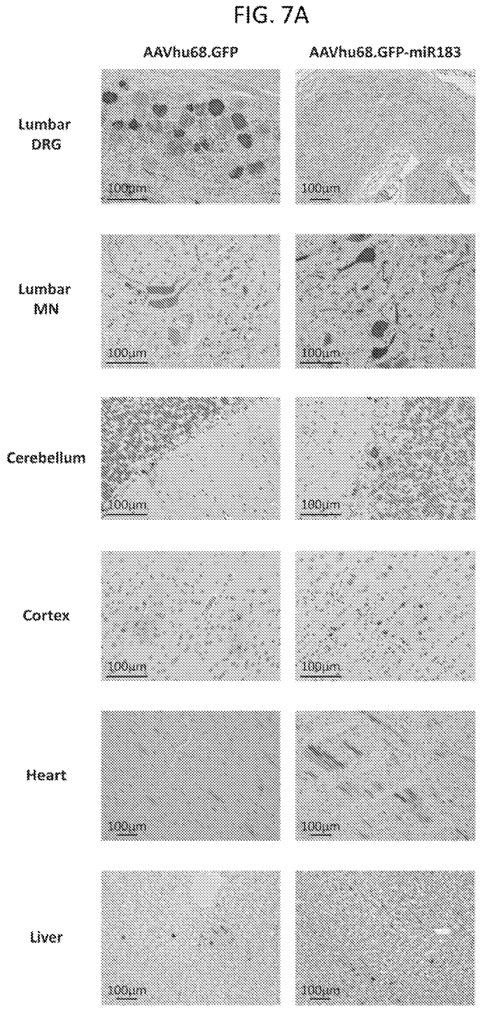

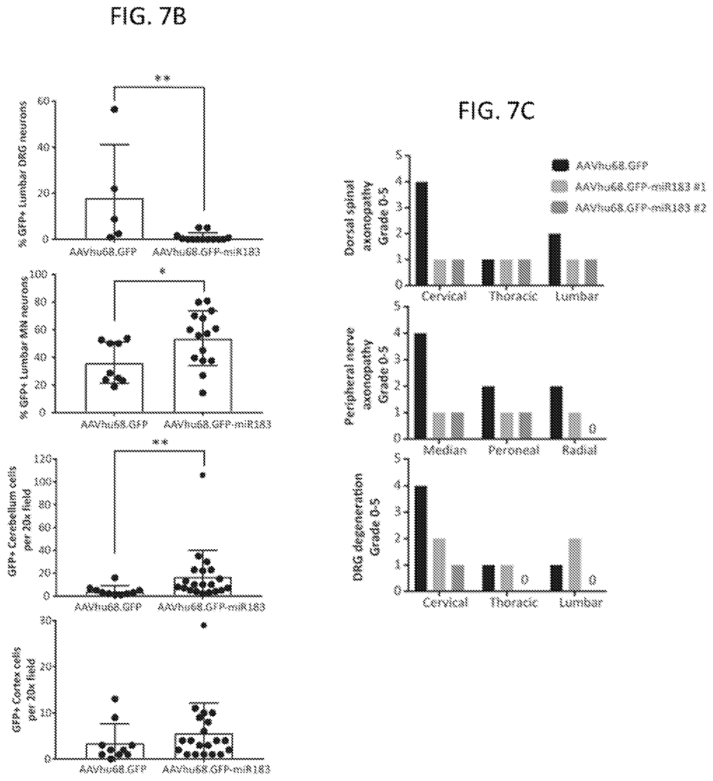

Based on the encouraging data in mice, we evaluated the GFP-miR183 target expression cassette in NHPs. We ICM-injected AAVhu68 vectors expressing GFP (n=2, 1 male, 1 female; age 5 and 8 years old, respectively) or GFP miR183 (n=4 female, age range 5-6 years old) from a CB7 promoter in rhesus macaques (3.5×10¹³ GC). Half of the animals were necropsied on day 14 for GFP expression. The remaining animals were necropsied on day 60 to evaluate expression and DRG toxicity. Animals tolerated the ICM-administered vector without clinical sequelae and there was no evidence of neuropathic pain for any of the animals enrolled in this study after vector administration. We observed a statistically significant reduction of GFP expression in DRG with the miR183-target containing vector compared with the control vector (p=0.0054), whereas expression was enhanced in the lumbar motor neurons (p=0.0273) and cerebellum (p=0.0044) and remained unchanged in cortex, heart, and liver (FIG. 7A and FIG. 7B). This was associated with a reduction of pathology across nine regions (DRG and dorsal spinal axonopathy at cervical, thoracic, and lumbar spine, and axonopathy of median, peroneal, and radial nerves). When the vector did not contain the miR183 targets, pathology was present in all regions and evenly distributed between grade 4, grade 2, and grade 1. The greatest degree of pathology with the miR183 vector was grade 2, which was present in only 11% of regions; the remaining regions included either grade 1 pathology (72%) or no pathology (17%; FIG. 7C).

We further evaluated miR183 target sequences in NHPs using vectors that expressed hIDUA—an enzyme deficient in patients with mucopolysaccharidosis I. Studies with this human transgene were the first published reports to highlight DRG toxicity in NHPs. The experiment included three groups: Group 1—control vector alone without miR183 targets (AAVhu68.CB7.CI.hIDUAcoV1.rBG) (n=3, 2 female, 1 male, age 2.5 years old); Group 2—control vector without miR183 targets in animals treated with steroids (prednisolone 1 mg/kg/day from day minus 7 to day 30 followed by progressive taper off; n=3, 3 male, age 2.5-3.5 years old); and Group 3—vector with miR183 targets (AAVhu68.CB7.CI.hIDUAcoV1.4×miR183.rBG) (n=3, 2 male, 1 female, age 2.25-2.5 years old). All vector genomes included an hIDUA coding sequence under the control of a chicken β-actin promoter and CMV enhancer elements (referred to as the CB7 promoter), a chimeric intron (CI) consisting of a chicken β-actin splice donor (973 bp, GenBank: X00182.1) and a rabbit β-globin splice acceptor element, and a rabbit β-globin polyadenylation signal (rBG, 127 bp, GenBank: V00882.1). The vector genome for ITR.CB7.CI.hIDUAcoV1.rBG.ITR is provided in SEQ ID NO: 14. The vector genome for ITR.CB7.CI.hIDUAcoV1.4×miRNA183.rBG.ITR is provided in SEQ ID NO: 16. All animals received an ICM injection of an AAVhu68 vector (1×10¹³ GC) and necropsies were performed at day 90 to evaluate transgene expression and DRG-related toxicity.

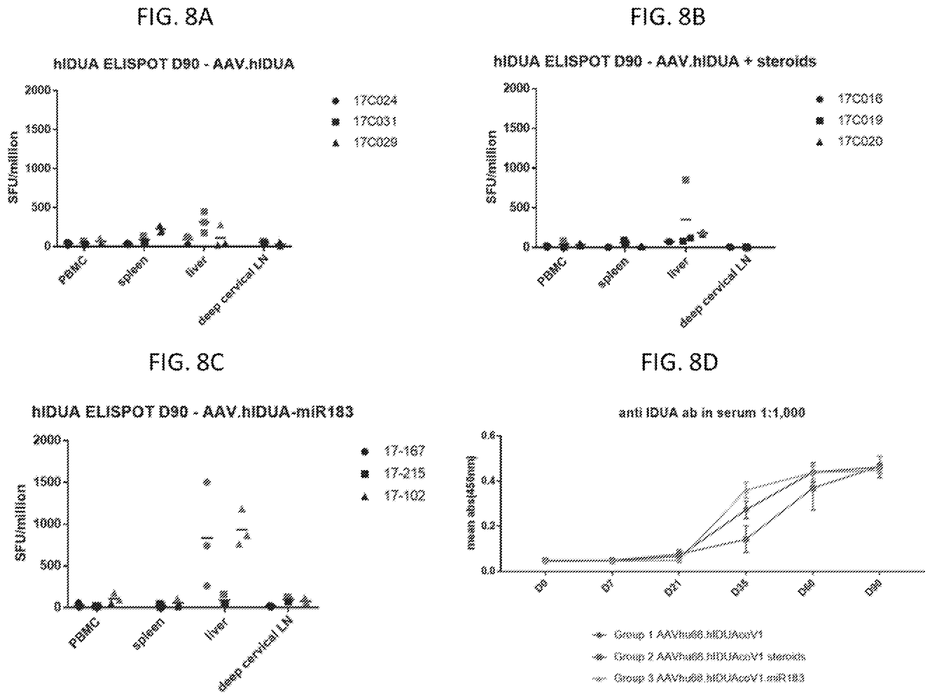

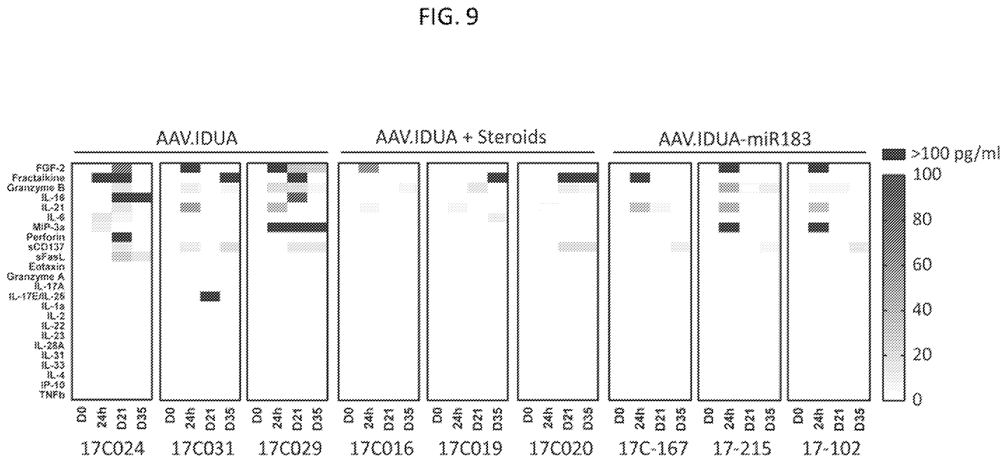

Animals from all groups tolerated ICM vector with no vector-related clinical findings or abnormalities in clinical pathology (Table 1 and Table 2). Pleocytosis in CSF was very low and limited to one animal in group 2 and one animal in group 3 (Table 3). We detected both T-cell responses (measured by ELISPOT) and antibodies to hIDUA in all three groups (FIG. 8A-FIG. 8D). Fractalkine and MIP-3a CSF quantities spiked at 24 hours post-vector administration, rising from undetected to >100 pg/mL in 2 animals from group 1 and 3 animals from group 3 whereas these analytes were undetectable at 24 hours but increased at 21 and 35 days in CSF from group 2 (prophylactic steroids). Undetectable or trace amounts (less than 15 pg/mL) of cytokines and chemokines were measured in the CSF of group 3 (hIDUA.miR183) animals at 21 and 35 days post-injection (when overexpression-induced stress would be expected), whereas one or several analytes (fractalkine, MIP-3a, IL16, perforin, and IL17) were >100 pg/mL in all animals from group 1 (FIG. 9).

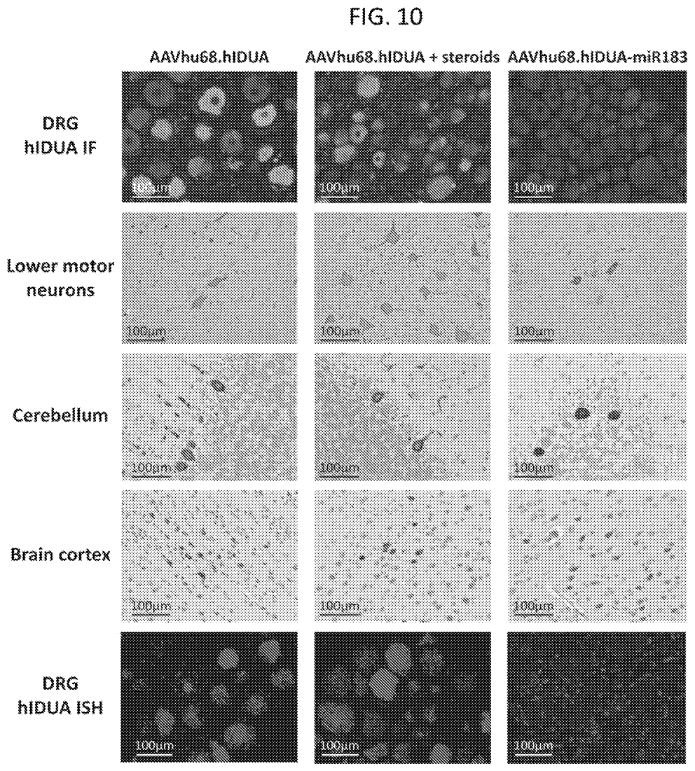

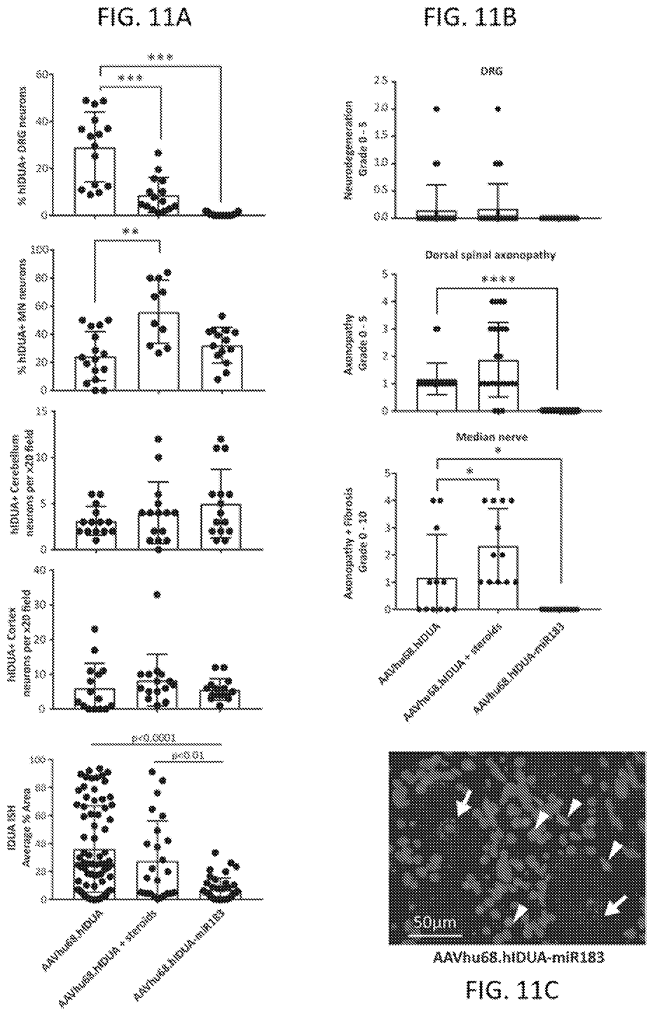

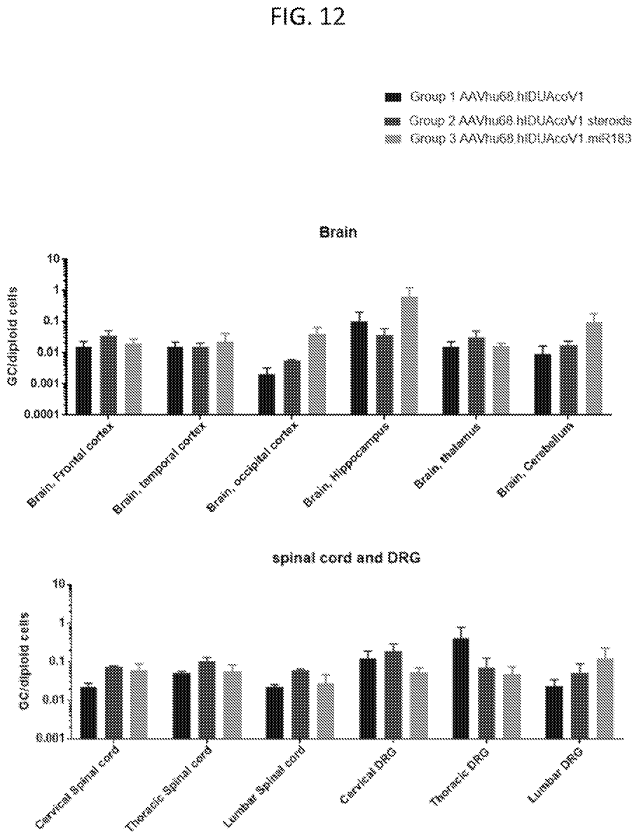

Using immunofluorescence and in situ hybridization (ISH), we observed high expression of hIDUA in DRG in groups 1 and 2, which used the control vector (without miR183 targets; FIG. 10 and FIG. 11A). We detected low to moderate hIDUA expression in other CNS compartments, including lower motor neurons of the spinal cord and cerebellum and cortical neurons (FIG. 10 and FIG. 11A). Incorporating the miR183 target into the vector (AAVhu68.hIDUA-miR183/group 3) ablated hIDUA protein expression in DRG neurons (p=0.000003) without decreasing expression in the CNS (spinal cord, cerebellum, and cortex) as highlighted by immunofluorescence (FIG. 10 top row, FIG. 11A) and immunohistochemistry (FIG. 10, FIG. 11A). At the mRNA level (FIG. 10, bottom row), cytoplasmic ISH signal in transduced DRG neurons was decreased from 42% of area in animals dosed with AAVu68.hIDUA to 7% in animals dosed with AAVhu68.hIDUA-miR183 (FIG. 11A), representing an 83% reduction. Reduction of hIDUA expression in DRGs was not due to decreased gene transfer since the biodistribution of vector throughout the CNS and DRGs was essentially the same across all groups (FIG. 12). Steroids moderately decreased expression in DRGs (p=0.0001) and increased it in lower motor neurons (p=0.0024) compared with the control vector (FIG. 10 and FIG. 11A). As expected, administration with the control vector (group 1) resulted in DRG, dorsal column, and peripheral nerve pathology that was comparatively milder than with GFP-expressing vectors. However, pathology was completely absent in the DRG (p=0.0583), dorsal column (p<0.0001) and peripheral (median) nerve (p=0.0137) of animals transduced with miR183 target-containing vector (group 3, FIG. 11B). Co-treatment with steroids (group 2) did not reduce toxicity of the parent vector (not containing miR183 targets) (FIG. 11B) but was instead associated with a trend of worsening toxicity in the peripheral nerve (p=0.0256) and dorsal column (p=0.066).

TABLE 1
Blood chemistry in NHP injected ICM with AAV.hIDUA vectors

| | | Total | | | | | | Alk | | Total | | |
|---|---|---|---|---|---|---|---|---|---|---|---|---|
| | | Protein | Albumin | Globulin | A/G | AST | ALT | Phosphatase | GGT | Bilirubin | BUN | Creatinine |
| Animal # | | g/ | g/ | g/ | Ratio | IU/ | IU/ | IU/ | IU/ | mg/ | mg/ | mg/ |
| and group | Timepoint | dL | dL | dL | — | L | L | L | L | dL | dL | dL |
| 17C024 | Baseline | 5.6 | 3.8 | 1.8 | 2.1 | 33 | 40 | 703 | 64 | 0.1 | 27 | 0.6 |
| AAVhu68.hIDUA | D 0 | 6.0 | 3.7 | 2.3 | 1.6 | 28 | 36 | 682 | 61 | 0.1 | 23 | 0.4 |
| | D 7 | 6.1 | 3.9 | 2.2 | 1.8 | 30 | 33 | 680 | 64 | 0.1 | 30 | 0.5 |
| | D 21 | 5.9 | 3.7 | 2.2 | 1.7 | 25 | 22 | 825 | 66 | 0.1 | 21 | 0.5 |
| | D 35 | 5.9 | 3.4 | 2.5 | 1.4 | 32 | 24 | 777 | 74 | 0.1 | 22 | 0.5 |
| | D 60 | 5.9 | 3.7 | 2.2 | 1.7 | 28 | 30 | 815 | 74 | 0.1 | 28 | 0.4 |
| | D 90 | 5.5 | 3.6 | 1.9 | 1.9 | 29 | 25 | 689 | 76 | 0.1 | 24 | 0.6 |
| 17C031 | Baseline | 5.7 | 3.8 | 1.9 | 2.0 | 35 | 39 | 539 | 36 | 0.1 | 27 | 0.5 |
| AAVhu68.hIDUA | D 0 | 6.5 | 4.0 | 2.5 | 1.6 | 37 | 43 | 570 | 47 | 0.2 | 23 | 0.4 |
| | D 7 | 6.3 | 3.9 | 2.4 | 1.6 | 27 | 39 | 547 | 45 | 0.1 | 26 | 0.5 |
| | D 21 | 6.6 | 3.9 | 2.7 | 1.4 | 33 | 34 | 476 | 39 | 0.1 | 25 | 0.6 |
| | D 35 | 6.3 | 3.4 | 2.9 | 1.2 | 32 | 31 | 591 | 42 | 0.1 | 24 | 0.5 |
| | D 60 | 6.2 | 4.0 | 2.2 | 1.8 | 32 | 33 | 545 | 45 | 0.1 | 25 | 0.4 |
| | D 90 | 6.0 | 3.6 | 2.4 | 1.5 | 34 | 27 | 478 | 43 | 0.1 | 24 | 0.5 |
| 17C029 | Baseline | 6.8 | 3.6 | 3.2 | 1.1 | 41 | 43 | 574 | 74 | 0.1 | 27 | 0.7 |
| AAVhu68.hIDUA | D 0 | 6.9 | 4.0 | 2.9 | 1.4 | 27 | 40 | 607 | 79 | 0.1 | 18 | 0.7 |
| | D 7 | 7.1 | 3.8 | 3.3 | 1.2 | 29 | 40 | 503 | 71 | 0.1 | 24 | 0.6 |
| | D 21 | 6.9 | 3.9 | 3.0 | 1.3 | 49 | 38 | 557 | 77 | 0.1 | 15 | 0.6 |
| | D 35 | 6.6 | 3.6 | 3.0 | 1.2 | 30 | 33 | 555 | 76 | 0.1 | 19 | 0.6 |
| | D 60 | 6.3 | 4.0 | 2.3 | 1.7 | 38 | 43 | 545 | 86 | 0.1 | 22 | 0.6 |
| | D 90 | 6.2 | 3.7 | 2.5 | 1.5 | 31 | 23 | 506 | 81 | 0.1 | 20 | 0.6 |
| 17C016 | Baseline | 6.0 | 3.9 | 2.1 | 1.9 | 42 | 35 | 623 | 59 | 0.2 | 16 | 0.5 |
| AAVhu68.hIDUA + | D 0 | 6.8 | 4.1 | 2.7 | 1.5 | 33 | 58 | 588 | 59 | 0.2 | 16 | 0.4 |
| steroids | D 7 | 6.3 | 3.9 | 2.4 | 1.6 | 26 | 28 | 444 | 51 | 0.1 | 20 | 0.5 |
| | D 21 | 6.4 | 3.7 | 2.7 | 1.4 | 29 | 23 | 393 | 44 | 0.2 | 16 | 0.6 |
| | D 35 | 6.9 | 3.9 | 3.0 | 1.3 | 32 | 29 | 364 | 41 | 0.1 | 21 | 0.5 |
| | D 60 | 6.5 | 3.8 | 2.7 | 1.4 | 41 | 29 | 525 | 63 | 0.2 | 23 | 0.5 |
| | D 90 | 5.7 | 3.6 | 2.1 | 1.7 | 32 | 29 | 752 | 79 | 0.1 | 12 | 0.5 |
| 17C019 | Baseline | 5.5 | 3.4 | 2.1 | 1.6 | 35 | 42 | 338 | 49 | 0.1 | 31 | 0.6 |
| AAVhu68.hIDUA + | D 0 | 6.3 | 3.7 | 2.6 | 1.4 | 32 | 39 | 383 | 49 | 0.1 | 19 | 0.6 |
| Steroids | D 7 | 6.1 | 3.6 | 2.5 | 1.4 | 26 | 34 | 322 | 47 | 0.1 | 30 | 0.7 |
| | D 21 | 6.0 | 3.3 | 2.7 | 1.2 | 28 | 32 | 332 | 53 | 0.1 | 19 | 0.7 |
| | D 35 | 5.9 | 3.5 | 2.4 | 1.5 | 42 | 38 | 378 | 49 | 0.1 | 27 | 0.6 |
| | D 60 | 5.6 | 3.6 | 2.0 | 1.8 | 35 | 36 | 437 | 61 | 0.2 | 23 | 0.5 |
| | D 90 | 5.6 | 3.6 | 2.0 | 1.8 | 41 | 34 | 650 | 73 | 0.2 | 21 | 0.6 |
| 17C020 | Baseline | 6.1 | 4.1 | 2.0 | 2.1 | 26 | 33 | 641 | 87 | 0.1 | 21 | 0.7 |
| AAVhu68.hIDUA + | D 0 | 6.5 | 4.1 | 2.4 | 1.7 | 21 | 25 | 538 | 61 | 0.1 | 12 | 0.6 |
| steroids | D 7 | 6.9 | 4.0 | 2.9 | 1.4 | 21 | 28 | 463 | 58 | 0.1 | 21 | 0.7 |
| | D 21 | 6.6 | 4.0 | 2.6 | 1.5 | 23 | 29 | 456 | 55 | 0.1 | 13 | 0.5 |
| | D 35 | 6.2 | 3.7 | 2.5 | 1.5 | 22 | 26 | 309 | 54 | 0.1 | 20 | 0.6 |
| | D 60 | 6.2 | 4.1 | 2.1 | 2.0 | 26 | 27 | 516 | 90 | 0.1 | 19 | 0.5 |
| | D 90 | 6.1 | 3.8 | 2.3 | 1.7 | 23 | 22 | 605 | 82 | 0.1 | 16 | 0.6 |
| 17-167 | Baseline | 6.6 | 3.9 | 2.7 | 1.4 | 24 | 21 | 764 | 112 | 0.1 | 18 | 0.5 |
| AAVhu68.hIDUA − | D 0 | 6.7 | 4.1 | 2.6 | 1.6 | 27 | 21 | 606 | 88 | 0.1 | 20 | 0.4 |
| miR183 | D 7 | 6.6 | 3.9 | 2.7 | 1.4 | 31 | 27 | 606 | 87 | 0.1 | 24 | 0.6 |
| | D 21 | 6.8 | 4.2 | 2.6 | 1.6 | 29 | 27 | 584 | 102 | 0.1 | 22 | 0.5 |
| | D 35 | 6.6 | 4.0 | 2.6 | 1.5 | 35 | 34 | 642 | 93 | 0.1 | 16 | 0.5 |
| | D 60 | 6.0 | 3.7 | 2.3 | 1.6 | 37 | 25 | 689 | 79 | 0.1 | 15 | 0.6 |
| | D 90 | 6.6 | 3.9 | 2.7 | 1.4 | 24 | 21 | 764 | 112 | 0.1 | 18 | 0.5 |
| 17-215 | Baseline | 6.1 | 3.4 | 2.7 | 1.3 | 24 | 34 | 536 | 51 | 0.1 | 22 | 0.5 |
| AAVhu68.hIDUA − | D 0 | 6.5 | 3.6 | 2.9 | 1.2 | 29 | 50 | 568 | 54 | 0.2 | 21 | 0.5 |
| miR183 | D 7 | 6.3 | 3.7 | 2.6 | 1.4 | 24 | 35 | 499 | 46 | 0.1 | 27 | 0.6 |
| | D 21 | 6.2 | 3.5 | 2.7 | 1.3 | 31 | 37 | 526 | 58 | 0.1 | 21 | 0.5 |
| | D 35 | 6.0 | 3.4 | 2.6 | 1.3 | 33 | 47 | 619 | 57 | 0.1 | 18 | 0.4 |
| | D 60 | 6.1 | 3.7 | 2.4 | 1.5 | 40 | 50 | 628 | 45 | 0.2 | 18 | 0.5 |
| | D 90 | 6.3 | 3.9 | 2.4 | 1.6 | 27 | 42 | 856 | 55 | 0.1 | 17 | 0.6 |
| 17-102 | Baseline | 6.7 | 3.8 | 2.9 | 1.3 | 24 | 31 | 613 | 57 | 0.1 | 20 | 6.7 |
| AAVhu68.hIDUA − | D 0 | 7.0 | 3.6 | 3.4 | 1.1 | 32 | 35 | 632 | 57 | 0.1 | 13 | 7.0 |
| miR183 | D 7 | 6.9 | 3.9 | 3.0 | 1.3 | 31 | 25 | 601 | 57 | 0.1 | 16 | 6.9 |
| | D 21 | 7.0 | 3.8 | 3.2 | 1.2 | 31 | 30 | 611 | 65 | 0.1 | 20 | 7.0 |
| | D 35 | 6.8 | 3.8 | 3.0 | 1.3 | 28 | 29 | 684 | 59 | 0.1 | 20 | 6.8 |
| | D 60 | 6.4 | 3.8 | 2.6 | 1.5 | 34 | 36 | 588 | 44 | 0.1 | 20 | 6.4 |
| | D 90 | 7.0 | 4.0 | 3.0 | 1.3 | 27 | 28 | 576 | 41 | 0.1 | 18 | 7.0 |

TABLE 2
Complete blood count in NHP injected ICM with AAV.hIDUA vectors

| | | WBC | RBC | | | Platelet | | | | | |
|---|---|---|---|---|---|---|---|---|---|---|---|
| Animal # | | ×10³/ | ×10⁶/ | HGB | HCT | ×10³/ | Neutrophils | Lymphocytes | Monocytes | Eosinophils | Basophils |
| and group | Timepoint | μL | μL | g/dL | % | μL | /μL | /μL | /μL | /μL | /μL |
| 17C024 | Baseline | 8.6 | 6.1 | 13.1 | 43 | 305 | 2838 | 5246 | 344 | 172 | 0 |
| AAVhu68.hIDUA | D 0 | 6.1 | 5.5 | 12.7 | 40 | 304 | 3294 | 2562 | 183 | 61 | 0 |
| | D 7 | 5.3 | 5.1 | 11.2 | 38 | 343 | 1060 | 3869 | 265 | 106 | 0 |
| | D 21 | 6.7 | 5.7 | 12.8 | 42 | 289 | 1742 | 4422 | 335 | 201 | 0 |
| | D 35 | 5.6 | 5.6 | 12.7 | 42 | 377 | 1792 | 3584 | 168 | 56 | 0 |
| | D 60 | 6.6 | 5.8 | 13.0 | 42 | 356 | 1716 | 4488 | 198 | 198 | 0 |
| | D 90 | 5.4 | 5.7 | 13.1 | 41 | 399 | 1674 | 3456 | 162 | 108 | 0 |
| 17C031 | Baseline | 11.9 | 5.3 | 12.6 | 41 | 386 | 4879 | 6188 | 476 | 357 | 0 |
| AAVhu68.hIDUA | D 0 | 5.5 | 5.2 | 12.5 | 41 | 366 | 2255 | 2970 | 165 | 110 | 0 |
| | D 7 | 14.4 | 4.8 | 11.2 | 39 | 357 | 8064 | 5040 | 864 | 432 | 0 |
| | D 21 | 10.7 | 5.4 | 12.8 | 43 | 410 | 5136 | 4815 | 428 | 321 | 0 |
| | D 35 | 9.7 | 5.3 | 12.8 | 43 | 267 | 4559 | 4559 | 388 | 194 | 0 |
| | D 60 | 8.3 | 5.3 | 12.7 | 42 | 390 | 2988 | 4731 | 332 | 249 | 0 |
| | D 90 | 8.4 | 4.9 | 12.1 | 39 | 414 | 4284 | 2940 | 672 | 420 | 84 |
| 17C029 | Baseline | 15.5 | 5.9 | 12.7 | 43 | 561 | 4495 | 9610 | 775 | 620 | 0 |
| AAVhu68.hIDUA | D 0 | 15.1 | 5.7 | 12.6 | 41 | 493 | 9362 | 4681 | 755 | 302 | 0 |
| | D 7 | 11.2 | 5.8 | 12.7 | 43 | 576 | 2800 | 7280 | 784 | 336 | 0 |
| | D 21 | 10.6 | 5.8 | 12.6 | 42 | 496 | 4982 | 4770 | 530 | 318 | 0 |
| | D 35 | 12.0 | 5.8 | 13.1 | 44 | 511 | 2520 | 8280 | 600 | 600 | 0 |
| | D 60 | 11.6 | 5.8 | 13.6 | 43 | 497 | 3480 | 7192 | 696 | 232 | 0 |
| | D 90 | 19.6 | 5.7 | 12.9 | 42 | 283 | 14896 | 3528 | 980 | 196 | 0 |
| 17C016 | Baseline | 10.2 | 5.9 | 13.5 | 44 | 235 | 1734 | 7752 | 306 | 408 | 0 |
| AAVhu68.hIDUA + | D 0 | 8.9 | 5.6 | 12.9 | 44 | 353 | 3382 | 5073 | 356 | 89 | 0 |
| steroids | D 7 | 9.6 | 5.5 | 12.3 | 41 | 346 | 2976 | 5952 | 576 | 96 | 0 |
| | D 21 | 11.9 | 5.4 | 12.4 | 41 | 424 | 2856 | 8330 | 595 | 119 | 0 |
| | D 35 | 7.9 | 5.4 | 12.7 | 43 | 352 | 4977 | 2528 | 316 | 79 | 0 |
| | D 60 | 8.7 | 5.6 | 12.8 | 43 | 380 | 2610 | 5655 | 348 | 87 | 0 |
| | D 90 | 8.4 | 5.4 | 12.7 | 41 | 357 | 2940 | 4956 | 420 | 84 | 0 |
| 17C019 | Baseline | 10.7 | 5.5 | 13.4 | 40 | 257 | 6099 | 4066 | 214 | 321 | 0 |
| AAVhu68.hIDUA + | D 0 | 10.6 | 6.4 | 13.9 | 49 | 358 | 7208 | 2650 | 318 | 424 | 0 |
| Steroids | D 7 | 10.8 | 5.9 | 13.1 | 44 | 286 | 3564 | 6048 | 1080 | 108 | 0 |
| | D 21 | 12.3 | 5.7 | 12.9 | 41 | 462 | 5658 | 5904 | 492 | 246 | 0 |
| | D 35 | 12.4 | 5.9 | 13.4 | 45 | 297 | 7812 | 3720 | 620 | 248 | 0 |
| | D 60 | 8.6 | 5.8 | 13.4 | 43 | 373 | 3612 | 4386 | 430 | 172 | 0 |
| | D 90 | 11.1 | 5.5 | 13.0 | 41 | 349 | 8214 | 2442 | 333 | 111 | 0 |
| 17C020 | Baseline | 14.6 | 6.5 | 14.1 | 47 | 357 | 4818 | 8760 | 584 | 438 | 0 |
| AAVhu68.hIDUA + | D 0 | 15.4 | 6.1 | 13.7 | 44 | 339 | 8778 | 5390 | 616 | 616 | 0 |
| steroids | D 7 | 14.0 | 6.0 | 13.4 | 43 | 378 | 4060 | 8680 | 840 | 420 | 0 |
| | D 21 | 14.4 | 5.7 | 12.9 | 42 | 327 | 6336 | 7056 | 720 | 144 | 144 |
| | D 35 | 11.1 | 6.2 | 13.9 | 45 | 369 | 2664 | 7770 | 444 | 222 | 0 |
| | D 60 | 10.8 | 6.2 | 14.0 | 45 | 380 | 4104 | 5940 | 324 | 432 | 0 |
| | D 90 | 7.2 | 5.9 | 13.5 | 44 | 296 | 2376 | 4536 | 216 | 72 | 0 |
| 17-167 | Baseline | 10.6 | 5.4 | 13.7 | 44 | 245 | 1802 | 8162 | 530 | 106 | 0 |
| AAVhu68.hIDUA − | D 0 | 7.2 | 5.1 | 12.2 | 40 | 373 | 936 | 5904 | 288 | 72 | 0 |
| miR183 | D 7 | 7.6 | 4.9 | 12.2 | 39 | 258 | 1140 | 6080 | 304 | 76 | 0 |
| | D 21 | 10.2 | 5.5 | 13.4 | 45 | 270 | 3468 | 5712 | 714 | 102 | 204 |
| | D 35 | 10.6 | 5.3 | 13.2 | 42 | 275 | 5406 | 4664 | 424 | 106 | 0 |
| | D 60 | 9.5 | 5.2 | 13.4 | 42 | 115 | 3515 | 5510 | 380 | 95 | 0 |
| | D 90 | 10.6 | 5.4 | 13.7 | 44 | 245 | 1802 | 8162 | 530 | 106 | 0 |
| 17-215 | Baseline | 12.9 | 5.8 | 12.7 | 44 | 380 | 2709 | 9546 | 387 | 258 | 0 |
| AAVhu68.hIDUA − | D 0 | 10.0 | 5.5 | 11.9 | 40 | 374 | 3600 | 5900 | 300 | 200 | 0 |
| miR183 | D 7 | 11.2 | 5.4 | 12.0 | 40 | 423 | 2800 | 7616 | 560 | 224 | 0 |
| | D 21 | 11.8 | 5.2 | 11.3 | 39 | 375 | 2242 | 9086 | 236 | 118 | 118 |
| | D 35 | 12.5 | 5.6 | 12.4 | 42 | 337 | 5125 | 6625 | 500 | 250 | 0 |
| | D 60 | 10.3 | 5.4 | 12.1 | 39 | 315 | 3811 | 5974 | 309 | 206 | 0 |
| | D 90 | 13.7 | 5.3 | 12.1 | 38 | 415 | 8494 | 4795 | 274 | 137 | 0 |
| 17-102 | Baseline | 5.3 | 6.0 | 14.0 | 46 | 518 | 530 | 3816 | 371 | 530 | 53 |
| AAVhu68.hIDUA − | D 0 | 12.4 | 5.9 | 13.5 | 44 | 478 | 6944 | 4588 | 496 | 372 | 0 |
| miR183 | D 7 | 9.2 | 5.8 | 13.1 | 44 | 501 | 3312 | 4968 | 368 | 552 | 0 |
| | D 21 | 9.0 | 5.9 | 14.0 | 45 | 510 | 2250 | 5940 | 540 | 270 | 0 |
| | D 35 | 9.4 | 6.4 | 14.5 | 49 | 409 | 3760 | 4888 | 564 | 188 | 0 |
| | D 60 | 9.1 | 6.2 | 14.6 | 46 | 410 | 3276 | 5005 | 455 | 364 | 0 |
| | D 90 | 7.8 | 5.9 | 14.4 | 44 | 528 | 3042 | 4212 | 312 | 234 | 0 |

TABLE 3
CSF white blood cell counts (cells per μL)
in NHP injected ICM with AAV.hIDUA vectors

| | Animal | Day | Day | Day | Day | Day |
|---|---|---|---|---|---|---|
| Group | # | 0 | 21 | 35 | 60 | 90 |
| AAVhu68.hIDUA | 17C024 | 0 | 1 | 2 | 1 | 2 |
| | 17C031 | 0 | 1 | 2 | 1 | 1 |
| | 17C029 | 0 | 0 | 0 | 0 | 0 |
| AAVhu68.hIDUA + | 17C016 | 0 | 0 | 0 | 0 | 3 |
| steroids | 17C019 | 0 | 1 | 2 | 1 | 0 |
| | 17C020 | 0 | 0 | 5 | 1 | Blood con- |
| | | | | | | tamination |
| AAVhu68.hIDUA − | 17-167 | 0 | 2 | 3 | 1 | 1 |
| miR183 | 17-215 | 0 | 1 | 2 | 1 | 1 |
| | 17-102 | 0 | 3 | 7 | 2 | 0 |

### AAV-Induced DRG Toxicity in NHPs Occurs Via Neuronal Apoptosis

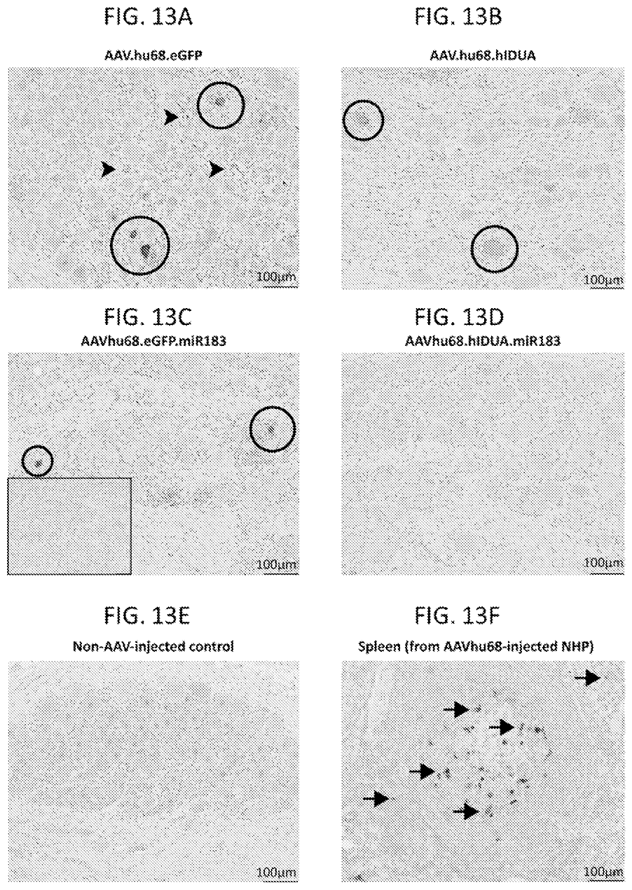

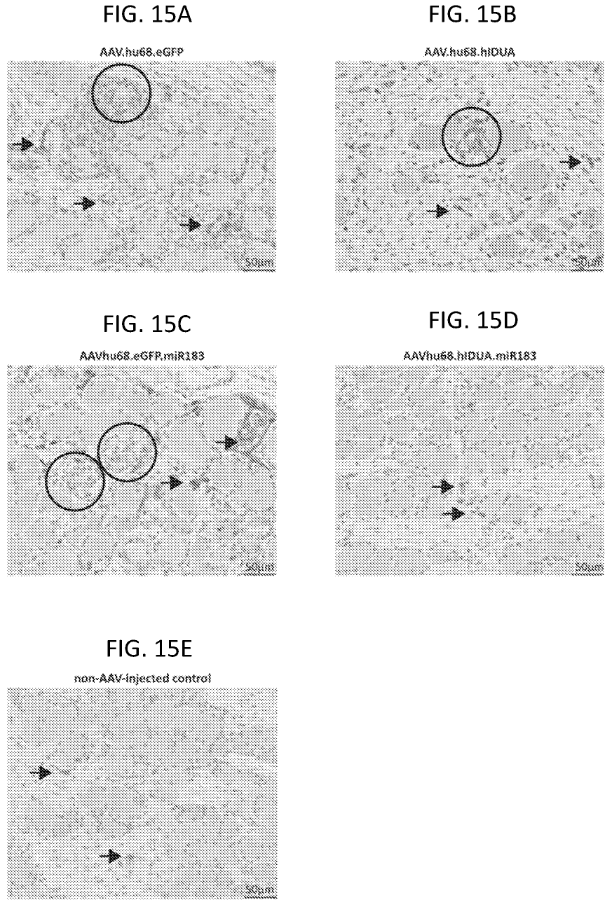

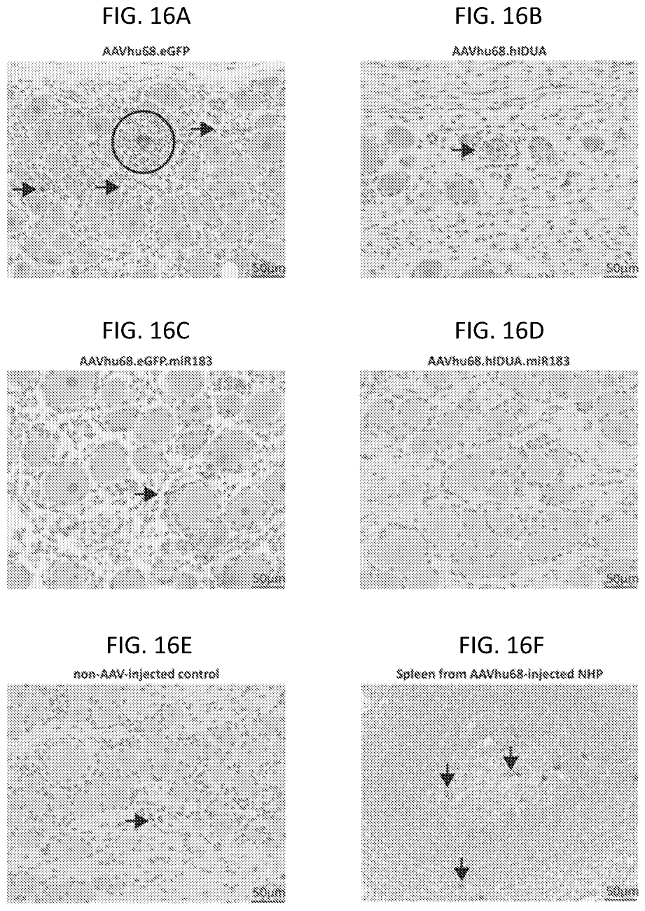

In order to investigate the mechanism of neuronal degeneration in DRG, we selected some histological sections from animals injected with vectors with and without miR183 and performed IHC for markers of cellular apoptosis and the unfolded protein response (UPR). Initial studies focused on the activation of caspase-3, which is a downstream marker of apoptosis. DRG of animals that exhibited neuronal degeneration based on haemotoxylin and eosin evaluation showed positive IHC staining for activated caspase-3 along with cellular infiltrates (FIG. 13A-FIG. 13C). DRG from a non-AAV-injected animal and spleen served as negative and positive controls, respectively (FIG. 13E and FIG. 13F). Caspase-3-positive neurons in DRGs were more abundant in the sections from the animal injected with AAVhu68.GFP (20 caspase-3 positive DRG neurons) as compared to the 3 animals injected with AAV.hIDUA (11, 0, and 1 positive DRG neurons) In each case, inclusion of the miRNA183 target sequence reduced the number of cells with activated caspase-3 (3 and 0 positive neurons with GFP.miR183, n=2 animals and 0 with hIDUA.miR183, n=3 animals; FIG. 13C-FIG. 13D). We investigated apoptosis induced by adaptive or innate immunity, in what is referred to as the extrinsic pathway, by evaluating upregulation of caspase-8 by IHC. Only the sections with caspase-3 positive neurons were processed. Degenerating neuronal cell bodies across all vector groups were negative for activated caspase-8, whereas infiltrating cells were strongly positive for caspase-8, which served as an internal positive control (FIG. 15A-FIG. 15E). The same sections were also evaluated for activated caspase-9, a common marker of the intrinsic pathway of apoptosis. This mechanism of apoptosis is mediated via the release of cytochrome C due to increased membrane permeability of the mitochondria and activation of caspase 9. IHC demonstrated caspase-9 in one degenerating neuronal cell body of DRG in an animal that received AAVhu68.eGFP (FIG. 16A); however, no caspase-9 was observed in animals that received AAVhu68.eGFP.miRNA and exhibited neuronal degeneration (FIG. 16C). There were no positive caspase-9 neurons from animals that received AAVhu68.hIDUA vectors with or without miR183, although this may have been a function of the decreased incidence of lesions observed with these vectors compared to AAVhu68.eGFP, which reduces the likelihood of finding neurons at the right stage of degeneration on histological sections (FIG. 16B and FIG. 16D).

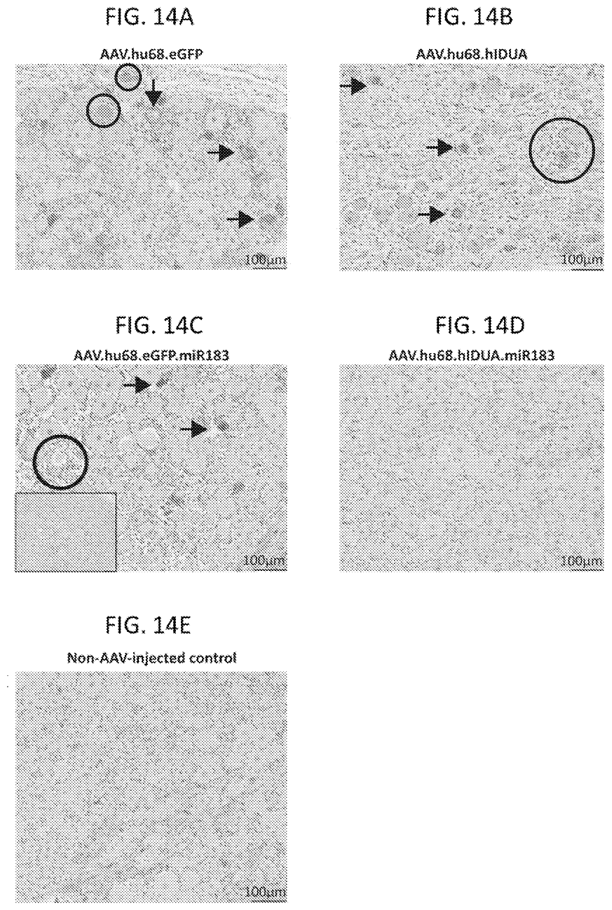

In order to support the proposed mechanism of toxicity due to protein overexpression of the transgene, we performed IHC for activating transcription factor 6 (ATF6) in 1 animal per group. The UPR triggers ATF6 activation in the Golgi to generate cytosolic fragments, which migrate to the nucleus to activate the transcription of ER-associated binding elements; apoptosis via the UPR occurs through the intrinsic pathway. IHC for ATF6 was multifocally positive in the cytoplasm of neuronal and perineuronal satellite cells in the DRG of animals that received AAVhu68.eGFP (>40 positive cells), AAVhu.68.hIDUA (>40 positive cells), and AAVhu68.eGFP.miR183 (18 positive cells), which corresponded to lesion severity (FIG. 14A-FIG. 14 C). By contrast, animals that received AAVhu68.hIDUA.miR183, as well as a naïve non-AAV-injected control NHP, were diffusely negative for ATF6 (FIG. 14D and FIG. 14E). Consistent with the overall study findings, animals that received vector with miR183 showed decreased positive ATF6 signal, indicating decreased cellular stress.

Toxicity of DRGs is likely to occur in any gene therapy that relies on high systemic doses of vector or direct delivery of vector into the CSF. This safety concern is limited to primates and usually manifests asymptomatically. However, DRG toxicity has the potential to cause substantial morbidity such as ataxia due to proprioceptive defects. The Food and Drug Administration recently put an intrathecal AAV9 trial for late-onset SMA on partial clinical hold due to NHP DRG toxicity, thus underscoring how this risk may limit the development of AAV therapies.

It was originally hypothesized that this toxicity was caused by destructive T-cell immunity to transduced neurons in DRGs directed towards foreign capsid or transgene epitopes. However, strong immune suppression regimens such as MMF and rapamycin did not prevent the toxicity in toxicology studies, nor did steroids in this study. The time course of delayed but non-progressive DRG degeneration did not support the notion that adaptive immunity played a role. If cytotoxic T cells were involved, we would have observed degeneration of DRG and of other cell types expressing the transgene, as well as mononuclear cell infiltrates that began early and progressed over time.

It may be that high levels of DRG transduction create cellular stress which leads to degeneration of highly transduced DRG neurons. Histological analysis demonstrated that degeneration was limited to DRG neurons that expressed the most transgene protein. Neuron degeneration was also associated with caspase-3 and -9 activation, thus suggesting that apoptosis was caused by an intracellular source of stress as opposed to being mediated by T cells. Reduction of DRG degeneration by cell-specific ablation of transgene expression via miRNA183 suggests that overexpression of the transgene-derived mRNA or protein rather than the capsid or vector DNA drives this process. The increased ATF6 staining in neurons and satellite cells in animals receiving vectors without the miR targets compared to controls with miR targets or naïve animals implicates the UPR, although the inciting mechanism may differ between non-secreted (GFP) versus secreted (IDUA) transgenes.

The time course of delayed but self-limited DRG neuronal degeneration is consistent with the notion that non-immune toxicity is restricted to a subset of highly transduced cells. It is unclear whether the DRG toxicity and axonopathy are reversible. After following adult animals for six months, we have not observed resolution of the pathology. The only experiment where we did not see DRG toxicity in NHPs following ICM injection was when the vector was administered to one-month old macaques that were necropsied 4 years later. It is possible that infant primates are resistant to DRG toxicity, or their DRG neurons have regenerative capacity, or the lesions regressed over this extended time period. Our findings support that DRG toxicity is caused by transgene overexpression, a type of neurotoxicity that has been reported previously in the CNS of NHP after direct intracerebral administration of AAV expressing hexosaminidase, a lysosomal enzyme deficient in Tay-Sachs disease. Therefore, the severity of DRG toxicity should be influenced by dose, promoter strength, and the nature of the transgene. However, we have yet to find a CNS-directed AAV where we can achieve effective doses of vector in mature primates without DRG toxicity.

It is not understood why sensory neurons are one of the most efficiently transduced cells in primates. DRGs are easily accessed by systemically administered vectors because they reside outside of the CNS and have porous, fenestrated capillaries. Systemic vector could also access DRG neurons via retrograde transport following uptake by peripheral axons. The anatomy of sensory neuronal compartments that reside within the intrathecal space may promote high transduction of vectors delivered into the CSF. Axons of DRG neurons in the dorsal roots are exposed to CSF, thus providing easy access to vector following ICM/LP administration. Open access of the subarachnoid space to the extracellular fluid of the DRG should allow direct contact of ICM/LP vector to neurons and other cells of the DRG. Selective suppression of transgene expression within DRG neurons via inclusion of a miR183 target sequence facilitated an analysis of transgene expression in other DRG-associated cells, which should not be influenced by this miRNA. ISH revealed transgene mRNA in surrounding glial satellite cells that could suggest direct transduction. The functional consequence of the presence of transgene mRNA in glial cells is unknown.

Selectively inhibiting vector transgene expression should reduce and potentially eliminate DRG toxicity. The key for achieving this involves designing a strategy to specifically extinguish expression in DRG neurons without affecting expression elsewhere. Currently, it is not possible achieve this specificity through capsid modifications or tissue-specific promoters. Including targets for miR183 into the vector achieved the desired result of reducing/eliminating DRG toxicity without affecting vector manufacturing, potency, or biodistribution. However, the dose window may be tight as miR183 and RISC are likely to be saturated by high GC numbers. Quantification of ISH in our study suggests that a reduction of 80% at the mRNA level in transduced DRG neurons is enough to suppress toxicity. Careful dose-range studies coupled with minimal-efficacy dose studies in animal models are imperative to establish the viability of our approach for any given transgene. Included in the hIDUA NHP study was a group that received non-miR183-targeted vector with concomitant steroids—a standard approach for mitigating immune-mediated toxicity in AAV trials. DRG toxicity was not reduced in the steroid-treated group. This experiment demonstrates the limitations of prophylactic steroids in AAV gene therapy.

The modularity of this approach for diminishing DRG toxicity suggests that it could be used in any AAV vector considered for CNS-directed gene therapy. It is possible that miR183 targets within the vector could divert miR183 molecules away from their normal targets, possibly perturbing the physiology of the cell. Evidence for this would be toxicity limited to heavily transduced cells that express miR183 (DRGs). We have not observed toxicity in NHPs treated with AAV miR183 target-containing vectors. The redundancy of the miR183 cluster with common targets for miR183, 182, and 96 may decrease this theoretical risk as suggested in complete cluster versus single miRNA knockout models. Furthermore, other cell types that are known to express miR183 (olfactory epithelium, retina, inner ear, activated immune cells) would not be efficiently transduced upon ICM or systemic AAV delivery. Considering the concerns raised by regulatory agencies for DRG toxicity, we believe it is prudent to incorporate a miRNA183 de-targeting strategy into CNS gene therapy programs. The main limitation of this strategy is mitigating DRG toxicity in diseases like neuronal forms of Charcot— Marie—Tooth in which DRG transduction is necessary to achieve a therapeutic effect.

In summary, we have developed an approach for mitigating AAV-induced DRG toxicity in NHPs. This approach could be tested across a broad array of AAV vectors in various therapeutic applications.

### Example 3: Comparison of Engineered Sequences Encoding hIDUA and Effect of miR183 Target Sequences on IDUA Activity and Expression

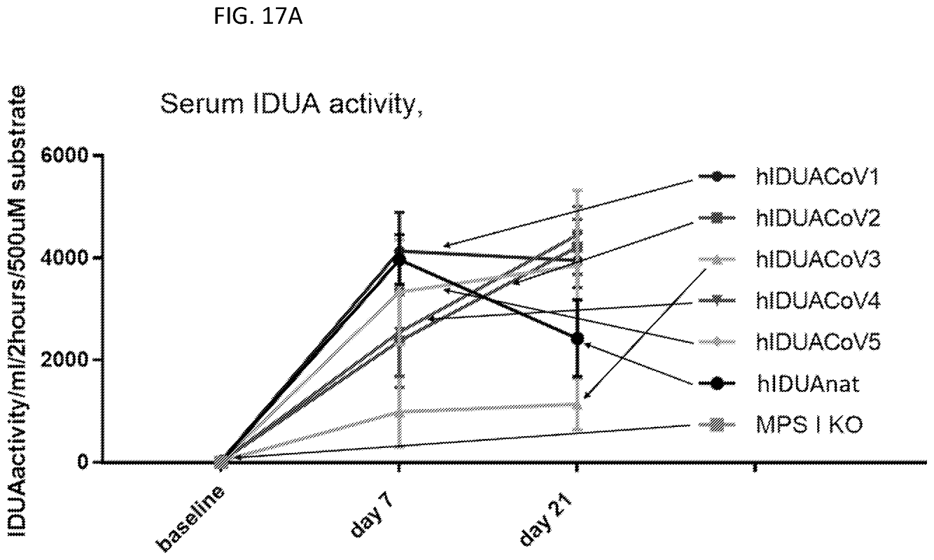

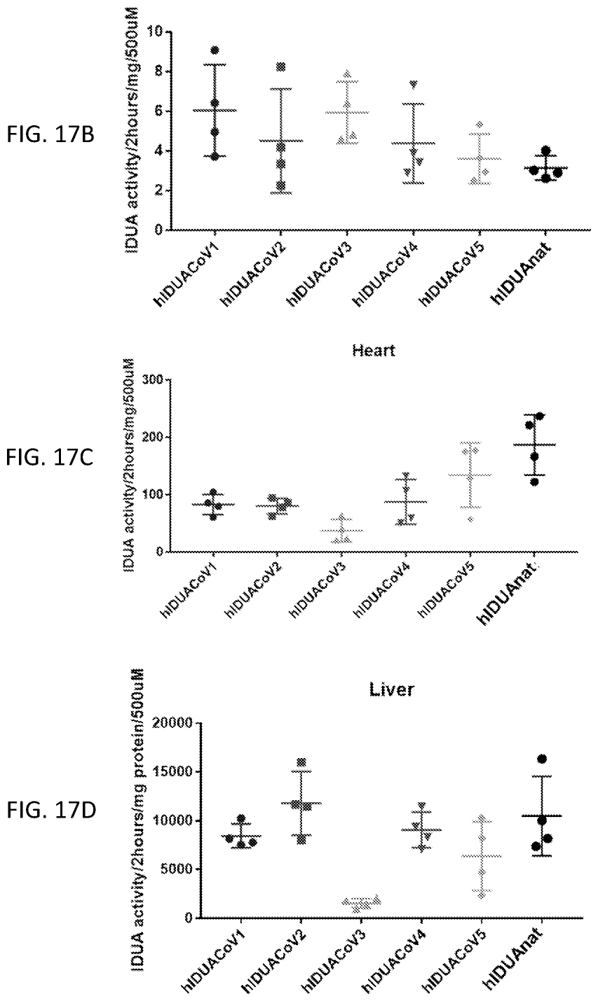

Wildtype male mice were injected IV with 1×10¹¹ GC of AAVhu68 for delivery of engineered sequences encoding human IDUA (SEQ ID NOs: 22-26), compared to the non-optimized natural cDNA. hIDUAcoV1 (SEQ ID NO: 22) showed the quickest and highest enzyme levels in serum and levels were stable at day 21 (FIG. 17A) suggesting no significant levels of anti-drug antibodies. hIDUAcoV1 was evaluated in further studies in part due to quick expression (serum day) and high levels of activity in the brain (FIG. 17B).

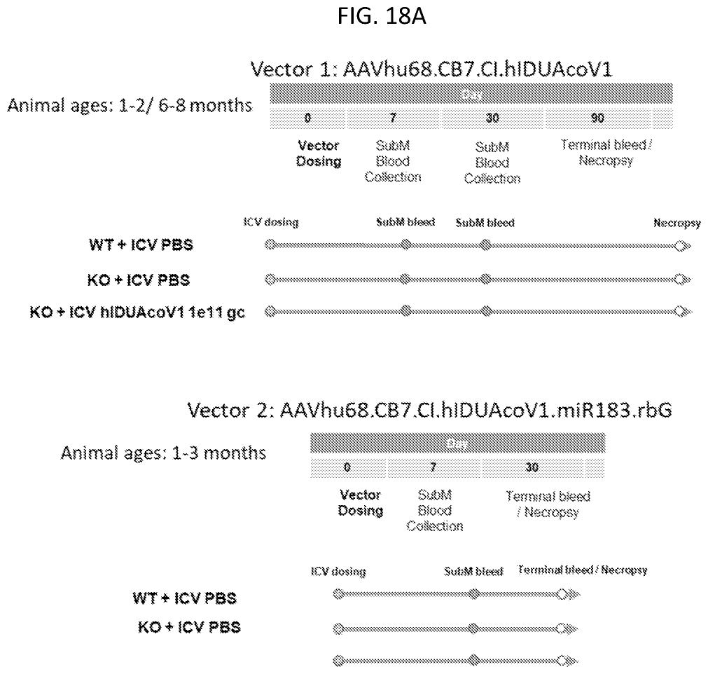

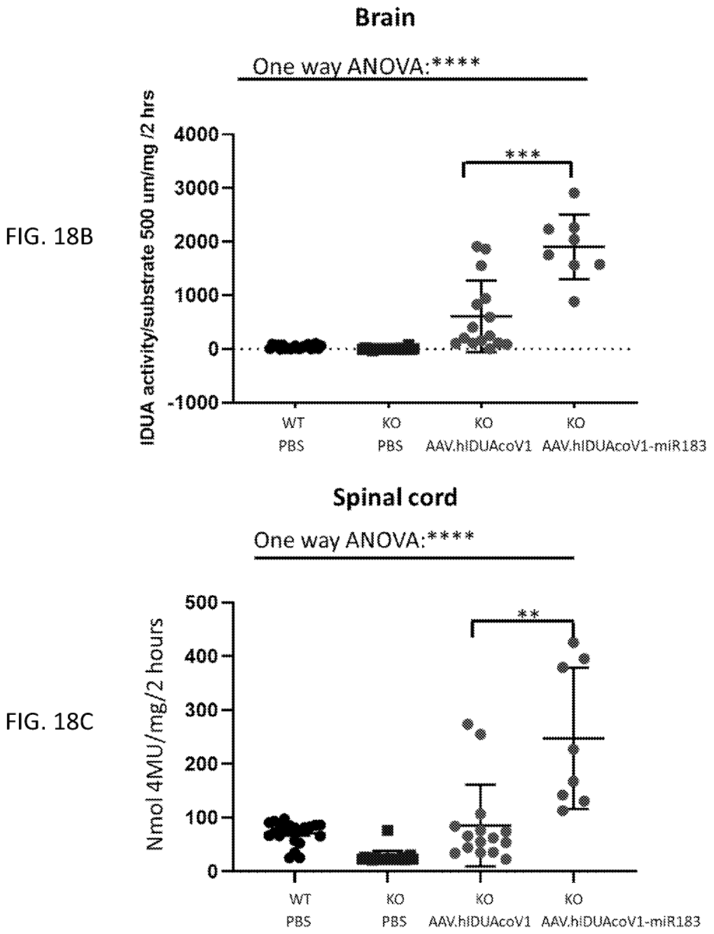

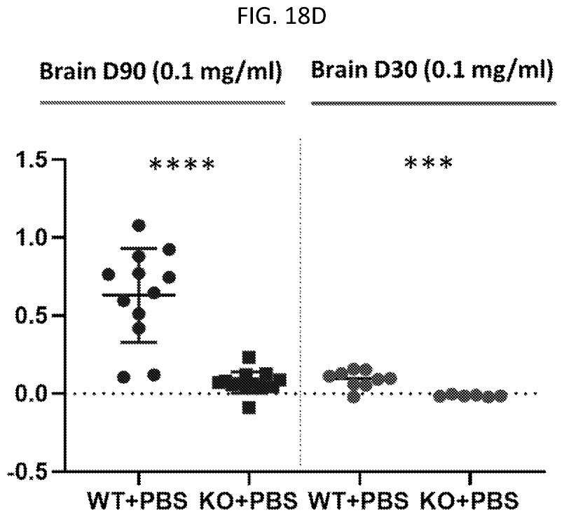

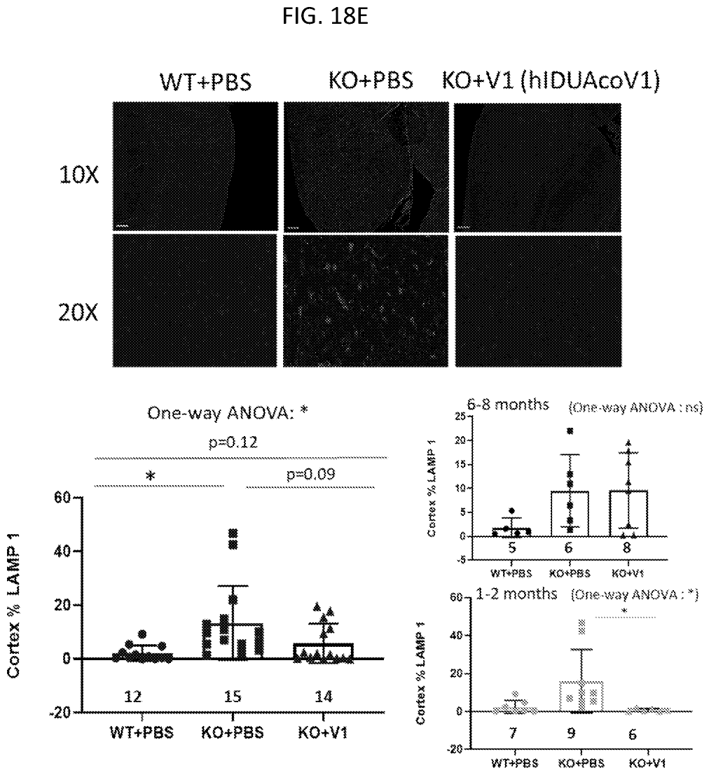

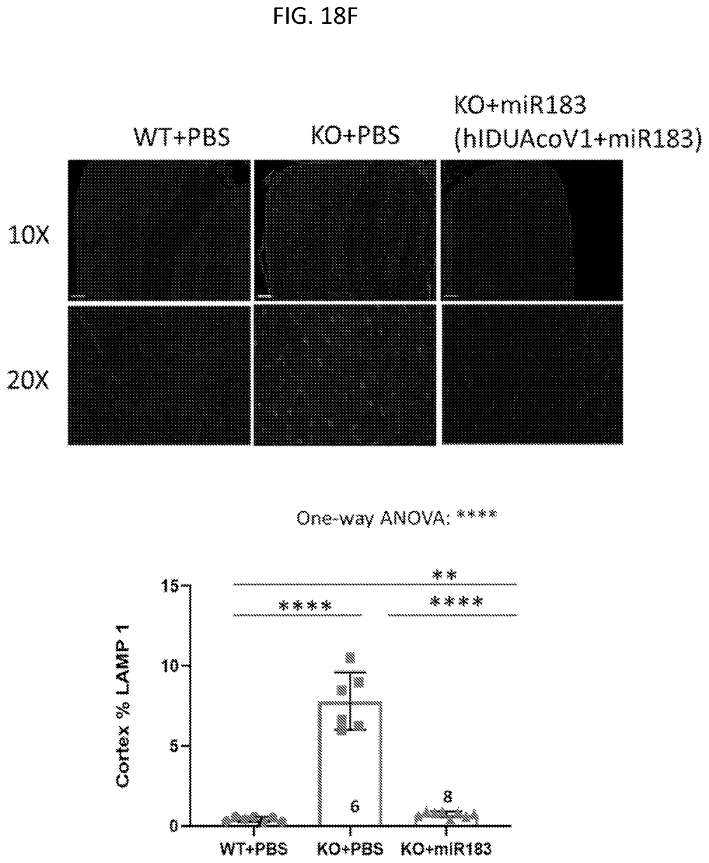

MPS I (IDUA-deficient) mice were injected ICV with 1×10¹¹ GC of AAVhu68 encoding hIDUACovl with or without miR183 targets (4× repeats). Mice were sacrificed 30 days or 90 days post injection (FIG. 18A and FIG. 18B). IDUA activity was above wildtype after ICV treatment with AAVhu68 encoding hIDUAcovl or hIDUAcovl-miR183 (FIG. 18C-FIG. 18C). Average levels were increased with the 4×miR183 target vector, indicating efficacy will be equal to or greater when miR183 targets are included in the construct. Tissues were processed to evaluate storage reduction using LAMP1 immunofluorescence as a marker of therapeutic efficacy. LAMP1 fluorescence was increased in KO mice treated with vehicle control and decreased in the cortex following AAV treatment with vectors encoding both versions of hIDUA with or without miR183 targets (FIG. 18E and FIG. 18F). Treatment efficacy was higher in young mice compared to older mice.

### Example 4: In vitro assessment of expression constructs with miR183 cluster target sequences

An in vitro assay is used to evaluate the activity and specificity of constructs harboring miRNA target sequences. As described in Example 2 above, HEK293 cells (or another suitable cell line) are co-transfected with a cis plasmid having the GFP transgene and plasmids expressing one or more miRNA, such as miR-182 and miR-183. The cis plasmids are designed with varying number of corresponding target miRNA sequences in the 3′UTR of the expression cassettes and alternative spacer sequences are introduced. At 72 hours post transfection, expression of GFP is quantified to determine relative levels of expression.

For example, constructs harboring one, two, three, four, or up to eight copies of target miR183 sequences are tested. The individual target sequences are directly linked or separated by spacer sequences, such as those provided in SEQ ID NOs: 5-7. Based on results of in vitro study, the suitable combination of sequences (including number of repeats) and spacers that reduce or eliminate expression of GFP are identified. Candidates from this study are then screened in vivo by delivering AAV vectors (e.g. AAV9 or AAV-PHP.B) having expression constructs with the same or similar arrangement of target miRNA sequences and spacers sequences. An exemplary in vivo mouse study to evaluate CNS expression levels, including, for example, detargeting of DRG (i.e. reduction of GFP expression), is provided in Example 2.

Similar studies are also performed using constructs having combinations of one, two, three, four, or up to eight copies of target sequences for miR182 with and without various spacer sequences. Additionally, constructs having combinations and different arrangements of miR182 and miR183 recognition sequences are generated. The constructs having miR182 target sequences only and combinations of miR182 and miR183 target sequences that show favorable reduced levels of expression in vitro are then evaluated in vivo, for example, following administration of AAV vectors to determine toxicity and levels of transgene expression (extent of detargeting) in cells of the CNS and DRG.

Alternatively, constructs are generated having one, two, three, four, or up to eight copies of a combination of miR182 target sequence and/or other mirl 83 cluster target sequences (i.e. a target sequences corresponding to miR-183, -96, or -182). The combination miR182-mir183 cluster target sequence-harboring constructs are tested in vitro using a GFP expression assay such as that described in Example 2 above. As above, the tested expression cassettes have various number of miRNA target sequences that are or are not separated by spacer sequences. The activity of certain constructs having combinations of miR182 target sequences and other mirl 83 cluster target sequences is then evaluated in vivo by generating AVV vectors that are then administered at high-dose IV. As above, expression of the AAV vector transgene is evaluated in various cells and tissues, including DRG and, in particular, in liver tissue.

Further, the effect of one, two, three, four, or up to eight copies of miR182 target sequences of transgene expression is evaluated. As above, experimental constructs for in vitro testing are generated introducing miR182 target sequences into the 3′UTR of an expression cassette. Where multiple miR182 sequences are introduced, the sequences may be consecutive or, alternatively, separated by any of various intervening spacer sequences. AAV vectors are generated having expression cassettes with any combination of miR182 target sequences and, where applicable, spacer sequences, and tested in vivo. In particular, in the case of expression cassettes having miR182 target sequences, transgene expression is evaluated in muscle tissue following high-dose IV administration of the AAV vector.

### Example 5: Delivery of an rAAV with miR Target Sequences Operably Linked to a Transgene does not Increase Expression of miR183 Cluster-Regulated Genes

Human CACNA2D1 and CACNA2D2 genes (members encode voltage-gated calcium channel) are predicted targets of the miR183 cluster (miR183/96/182) and a significant inverse correlation has been observed between all three miRNAs and CACNA2D1 and CACNA2D2 expression in DRG from human donors. See, e.g., Peng at al, “mirR-183 cluster scales mechanical pain sensitivity by regulating basal and neuropathic pain genes”. Science. 2017 Jun. 16; 356(6343):1168-1171. doi: 10.1126/science.aam7671. Epub 2017 Jun. 1. It has been reported that miR183 downregulates CACNA2D expression. However, increased expression of CACNA2D is expected if a “sponge effect” is present, which contributes to an increased sensitivity of animals to pain and pressure.

Stock rAAV containing a vector genome comprising eGFP with or without 4×miR183 target sequences or containing a vector genome comprising hIDUA with or without 4×miR183 were diluted to 2.5×10¹²/mL with rat-DRG medium, and 0.25 ml was added to each DRG-containing well of a 24-well-plate, after removing the old media. After 24 hours, media were removed and replaced with fresh media. The transductions were done in triplicates i.e. 3 wells for AAV-GFP and 3 for AAV-GFP-miR183 (2 wells for Mock control). To enhance transductions, adenovirus ADS (SignaGen Laboratories; Rockville, Md.) was also added at an MOI of 10, along with the AAV vectors. RNA was isolated separately for each well and used for q-RT-PCRs (one reaction/well; in duplicates). Total RNA was extracted from the DRG cultures 72 hours following transductions.

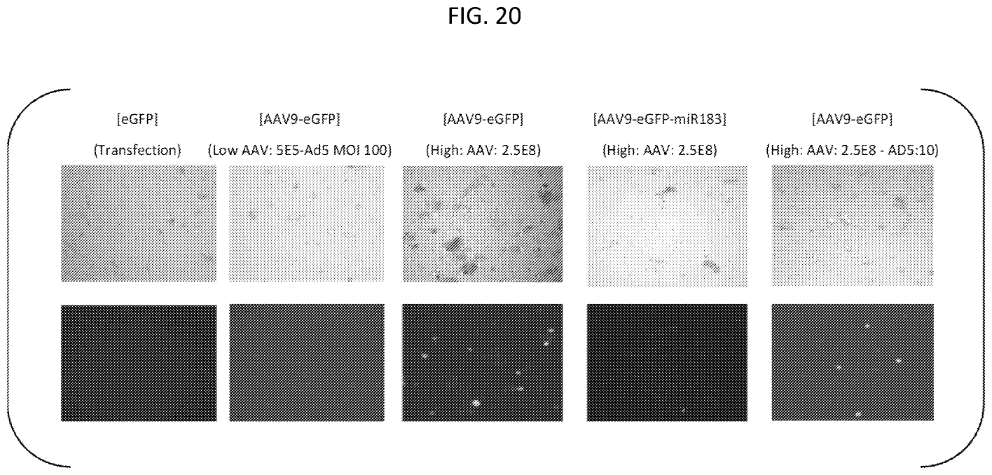

Expression levels of miR183 and the potential sponge effect on the target genes CACNA2D1 and CACNA2D2 were determined using primers specific to Rat CACNA2D1 (Assay ID Rn01442580) and CACNA2D2 (Assay ID: Rn00457825). FIG. 20 shows results of AAV transduction (AAV9) of various vectors carrying an eGFP transgene with or without four copies of the miR183 detargeting sequences at low (5×10⁵) or high (2.5×10⁸) concentration. The low and high dose without miR183 was tested with or without adenovirus type 5 (Ad5) helper co-transfection at a multiplicity of infection (MOI) of 100 (for low dose AAV9-eGFP or 10 (high dose AAV9-eGFP). All DRG neurons are transduced, no visible sign of toxicity is observed. No GFP expression is seen in DRG neurons, while some expression is observed in fibroblast like cells. This confirms repression of GFP transcription with the (×4)miR183 targets expression cassettes.

miR183 Sponge-Effect Study in NHP

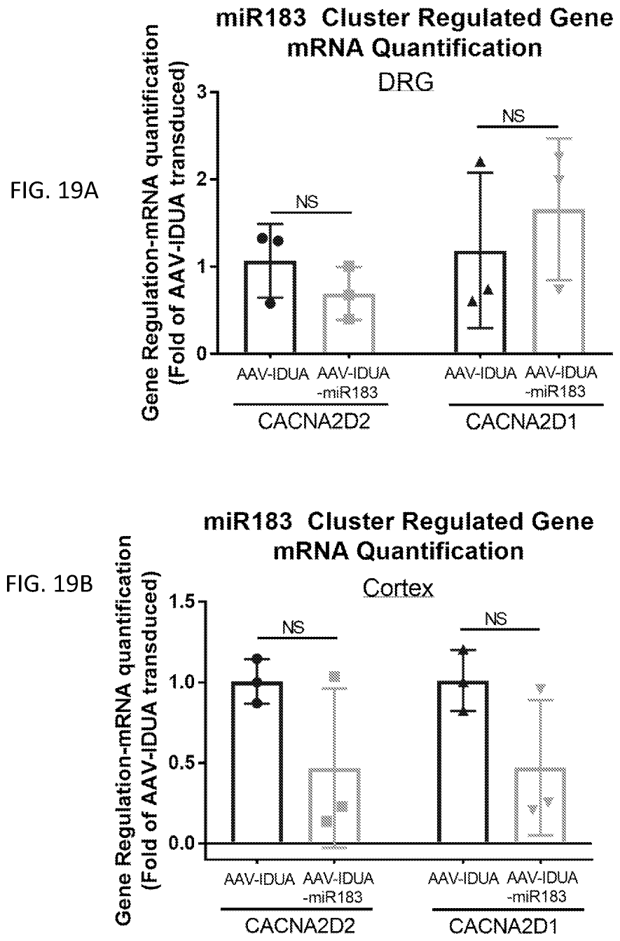

DRG (lumbar) and brain (frontal cortex) tissues were obtained from a non-human primate (NHP) rhesus monkey study (19-04) in which animal were treated with AAV-IDUA or AAV.hIDUA.4×mir183 vectors (n=3/group). A miRNeasy Mini Kit was used for total RNA isolation (Qiagen, Germantown, Md.) and the extracted RNA were then reverse transcribed with TaqMan™ MicroRNA Reverse Transcription Kit (Applied Biosystems), according to the instruction of the protocol. Quantitative real-time polymerase chain reaction (qPCR) was performed to determine the abundance of miR183 in different tissues, using the TaqMan MicroRNA Assay kit with primers specific to hsa-miR-183-5p (Assay ID 002269) and RNU6B (Assay ID 00193) (Applied Biosystems Inc., Foster City, Calif., USA) following the manufacturer's instructions. Similarly, the abundance of two of the direct targets of miR183, namely CACNA2D1 and CACNA2D2, was measured using the TaqMan Gene Expression Assay kit with primers specific to CACNA2D1 (Assay ID Hs00984840) and CACNA2D2 (Assay ID: Hs01021049), respectively. Each qPCR assay was conducted in triplicate using cDNA derived from 100 ng total RNA from a biological replicate and analyzed by the comparative threshold cycle (Ct) method. The average expression level of miR183 was normalized with RNU6B as an endogenous control gene, and the average level of CACNA2D1 and CACNA2D2 were normalized with GAPDH, using the 2^{-ΔΔct} method (Schmittgen T D, Livak K J. Analyzing real-time PCR data by the comparative C(T) method. Nat Protoc. 2008; 3(6):1101-8.). See, FIG. 19A (drg) and FIG. 19B (cortex). There was no increased expression of miR183 cluster-regulated genes (CACNA2D1 or CACNA2D2) when comparing AAV-IDUA or AAV-IDUA-miR183 animals in either DRG (high miR183 abundance) or frontal cortex (low miR183 abundance)

### Rat Neonatal Dorsal Root Ganglion (DRG) Neuron Cell Culture

Rat DRG neurons (Lonza Walkersville, Inc.) were thawed and added to 7 mL of recommended media (PNGM BulletKit: Primary Neuron Basal Medium containing 2 mM L-glutamine, 50 μg/ml Gentamicin/37 ng/ml Amphotericin, and 2% NSF-1). The 8 ml media containing ˜5.0×10⁵ DRG neurons was then divided between 8 wells of a 24-well tissue culture plate that was coated with poly-D-lysine (30 μg/ml; Sigma) immediately before adding the cells. Cells were incubated for 4 hours in a 37° C., 5% CO₂ incubator and then the media was removed and replaced with fresh, pre-warmed medium. To inhibit Schwann cell proliferation, mitotic cell inhibitors (5 μl of 17.5 ug/ml uridine and 5 ul of 7.5 μg/mL 5-fluoro-2-deoxyuridine/ml of medium) were added after the initial 4 hour incubation. Cells were incubated at 37° C., 5% CO₂ with complete media change on day 5 and 50% media change every 3 days after that. After 6 days of initial culture, Rat DRG neurons were transduced with AAV vectors as described above.

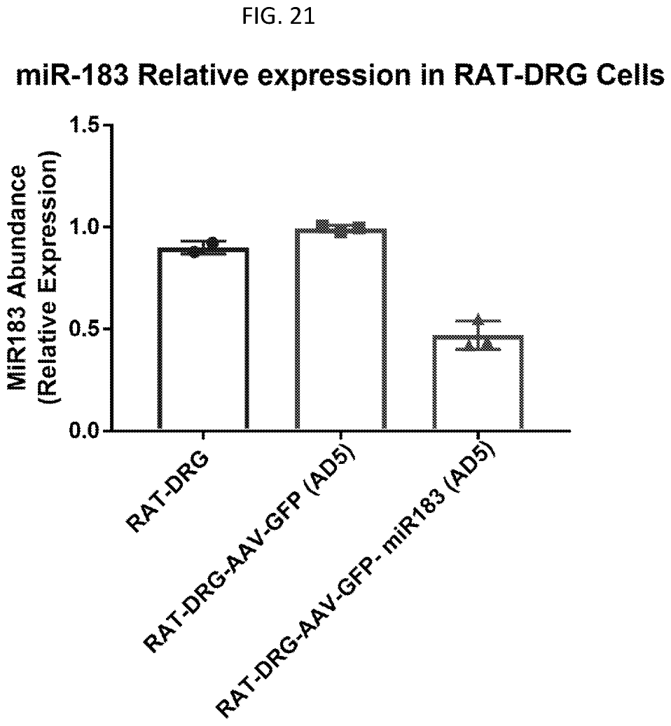

FIG. 21 shows the effect of the miR183 sponge effect study in rat DRG cells. miR183 levels in rat DRG cells were reduced when cells were transduced with the AAV9-eGFP-mir183. AAV9-eGFPmiR183—shows target engagement on the GFP-miR183 mRNA.

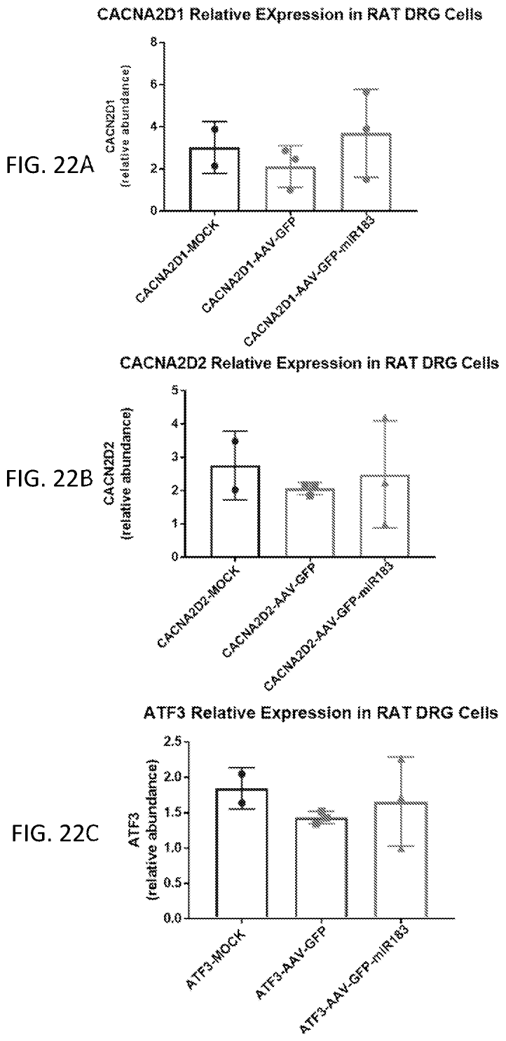

FIG. 22A-FIG. 22C show the effects in rat DRG cells on known miR183 regulated transcripts. FIG. 22A shows CACANA2D1 relative expression in rat DRG cells following delivery of a mock vector, AAV-GFP, or AAV-GFP-miR183 vector. FIG. 22B shows CACANA2D2 relative expression in rat DRG cells following delivery of a mock vector, AAV-GFP, or AAV-GFP-miR183 vector. FIG. 22C shows ATF3 expression in rat DRG cells following delivery of mock vector, AAV-GFP, or AAV-GFP-miR183 vector. There were no changes in relative expression of mRNA levels of these three known miR183-regulated transcripts. No difference was observed compared to untransduced mock wells and GFP-miR183 transduced wells. These data demonstrate the absence of a sponge effect in these cells. It is possible that either remaining levels of miR183 are sufficient or other members of the cluster (miR96 and/or miR182) can compensate for the decreased availability of miR183.

### Example 6: Meta-Analysis of DRG Pathology

The administration of adeno-associated virus (AAV) vectors to nonhuman primates (NHP) via the blood or cerebral spinal fluid (CSF) can lead to dorsal root ganglion (DRG) pathology. The pathology is minimal to moderate in most cases, clinically silent in affected animals, and characterized upon histopathological analysis by mononuclear cell infiltrate, neuronal degeneration, and secondary axonopathy of central and peripheral axons. We aggregated data from 33 nonclinical studies in 256 NHP and performed a meta-analysis of the severity of DRG pathology between different routes of administration, dose, time course, study conduct, age of the animals, sex, capsid, promoter, capsid purification method, and transgene. DRG pathology was observed in 83% of NHP with administration of AAV to the CSF, and 32% of NHP via the intravenous (IV) route. We showed that dose and age at injection significantly affected the severity while sex had no impact. DRG pathology was absent at acute time-points (i.e., <14 days), similar from 1 to 5 months post-injection, and less severe after 6 months. Vector purification method had no impact, and all capsids and promoters that we tested caused some DRG pathology. The data presented here from 5 different capsids, 5 different promoters, and 20 different transgenes suggest that DRG pathology is almost universal after AAV gene therapy in nonclinical studies using NHP. None of the animals receiving a therapeutic transgene displayed any clinical signs. Incorporation of sensitive techniques such as nerve conduction velocities can show modifications in a minority of animals that correlate with the severity of peripheral nerve axonopathy. Monitoring of sensory neuropathies in human CNS trials and high dose IV studies seems prudent to determine if clinically meaningful DRG pathology occurs.

### Materials and Methods

### Data Availability Statement

Aggregated data are presented with experimental details provided except for the specific transgenes which are proprietary to the sponsors that funded the work.

### Animals

237 rhesus macaques and 19 cynomolgus macaques from 33 studies were included in this meta-analysis. All animal procedures were approved by the Institutional Animal Care and Use Committee of the University of Pennsylvania. Rhesus macaques (Macaca mulatta) or Cynomolgus macaques (Macaca fascicularis) were procured from Covance Research Products, Inc. (Alice, Tex.), Primgen/Prelabs Primates (Hines, Ill.), MD Anderson (Bastrop, Tex.), or were donated. Animals were housed in an Association for Assessment and Accreditation of Laboratory Animal Care (AAALAC) International-accredited Nonhuman Primate Research Program facility at the University of Pennsylvania or at Children's Hospital of Philadelphia in stainless steel squeeze-back cages. Animals received varied enrichments such as food treats, visual and auditory stimuli, manipulatives, and social interactions.

### Test or Control Article Administration

For CSF administration, NHP received vectors diluted in sterile artificial CSF (vehicle) injected into the cisterna magna, under fluoroscopic guidance as previously described (N. Katz, et la. Hum Gene Ther Methods 29, 212-219, 2018). Lumbar puncture was performed under fluoroscopic guidance in anesthetized animals. After inserting a spinal needle into the L4-5 or L5-6 space, we confirmed placement by CSF return and/or by injecting up to 1 mL of contrast material (Iohexol 180). For intravenous administration, a catheter was placed in the saphenous vein and vector diluted in sterile lx Dulbecco's phosphate-buffered saline.

### Nerve Conduction Velocity Testing

Animals were sedated with a combination of ketamine/dexmedetomidine and placed in lateral or dorsal recumbency on a procedure table, with heat packs to maintain body temperature. The stimulator probe was positioned over the median nerve with the cathode closest to the recording site, and two needle electrodes inserted subcutaneously on digit II at the level of the distal phalanx (reference electrode) and proximal phalanx (recording electrode), while the ground electrode was placed proximal to the stimulating probe (cathode). A pediatric stimulator delivered the stimulus that we increased in a step-wise fashion until the peak amplitude response was reached. Up to 10 maximal stimuli were averaged and reported for the median nerve. The distance (cm) from the recording site to the stimulation cathode was measured and used to calculate the conduction velocity. Both the conduction velocity and the average of the sensory nerve action potential (SNAP) amplitude were reported.

### Vectors

For research studies, AAV vectors were produced and titrated as described previously (M. Lock et al. Hum Gene Ther 21, 1259-1271, 2010; M. Lock, et al. Hum Gene Ther Methods 25, 115-125, 2014). Briefly, HEK293 cells were triple-transfected and the culture supernatant was harvested, concentrated, and purified with an iodixanol gradient. For Good Laboratory Practice (GLP)-compliant toxicology studies, vector was also produced by triple-transfection of HEK293 cells and purified by affinity chromatography using a POROS™ CaptureSelect™ AAV9 resin (Thermo Fisher Scientific, Waltham, Mass.) as previously described (J. Hordeaux, et al. Mol Ther Methods Clin Dev 10, 79-88, 2018).

### Histopathology

For the majority of studies, a board-certified veterinary pathologist, initially blinded to test article/treatment groups, established severity scores defined as 0 for absence of lesion, 1 for minimal (<10%), 2 as mild (10-25%), 3 for moderate (25-50%), 4 for marked (50-95%), and 5 for severe (>95%). These scores were based on microscopic evaluation of hematoxylin and eosin (H&E)-stained tissues in which % represents the proportion of tissue affected by the lesion in an average high-power field. For all GLP and some non-GLP studies, peer-review was completed by an external board-certified veterinary pathologist. Severity scores from DRG degeneration and dorsal axonopathy of the spinal cord were established from cervical, thoracic, and lumbar segments; however, the number of sections evaluated varied across studies. In some studies, scores were assigned to individual sections of DRG and spinal cord when multiple tissue sections were present on a slide for a given segment; these were averaged for a single representative score. We consider spinal cord axonopathy as a better indicator of DRG pathology as it represents the collation of axons coming from all the DRG. We define DRG pathology as histopathological findings within the DRG cell bodies and spinal cord or spinal cord alone throughout this manuscript. Peripheral nerve axonopathy grades were established based on evaluation of the median (proximal and/or distal), radial, ulnar, sciatic (proximal and/or distal), peroneal, tibial, and/or sural nerves. When evaluation was performed on the proximal and distal median nerve, the proximal segment corresponded to the portion of nerve from the brachial plexus to the elbow and the distal segment corresponded to the portion of nerve from the elbow to the palm of the hand. When present, a severity score was given for periaxonal (i.e., endoneurial) fibrosis in peripheral nerves. For studies when peripheral nerves were evaluated bilaterally, axonopathy and periaxonal scores were averaged for each nerve.

### Data Extraction

The raw data including pathology scores and all pertinent study information were extracted from study files and aggregated in a single Excel spreadsheet. Two persons independently extracted and sorted the scores based on pre-determined search criteria to generate graphs and perform statistics. In case of discrepancy between the extracted outputs, consensus was reached upon collegial quality control.

### Statistics

For each parameter (i.e., age at injection, capsid, route of administration, time course, promoter, sex, vector purification method, and dose), we carried out comparisons of the pathology score between each pair of groups for each DRG or SC segment (i.e., cervical, thoracic and lumbar), using Wilcoxon rank-sum test with function “wilcox.test” within the R Program (version 3.5.0; https://cran.r-project.org).We then calculated combined p-values from the 3 comparisons for overall DRG or SC inter-group comparisons using Fisher's method with function “sumlog” in the “metap” package in R. Statistical significance was assessed on combined p-value at the 0.05 level.

### Results

### DRG Pathology Assessment

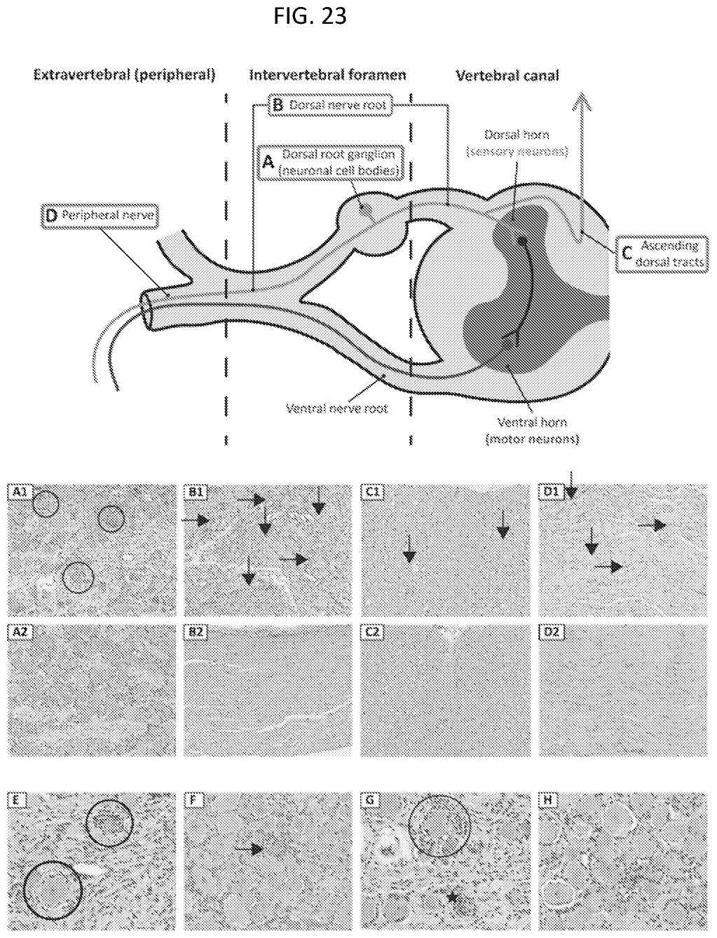

We developed a method to accurately evaluate and score lesions to DRG neurons based on neuro-anatomy and systematic evaluation of neurons and their corresponding axons. Neuronal cell bodies of primary sensory neurons are ovoid swellings at the base of each spinal dorsal root in the subarachnoid space located within the DRG. DRG neurons are pseudo-unipolar with one peripheral branch extending into the peripheral nerve and one central branch ascending dorsally in the spinal cord white matter tracts (FIG. 23). It is our experience that neuronal degeneration does not affect DRG uniformly, meaning multiple DRG from cervical, thoracic, and lumbar regions need to be collected to provide a representative sample. Pathology in the DRG manifests as mononuclear cell infiltration involving mononuclear inflammatory cells and proliferating resident satellite cells, with neuronal degeneration becoming visible at a later stage (FIG. 23, A1 circles). Secondary to neuronal cell body injury is axonal degeneration (i.e., axonopathy) along DRG axonal projections in the nerve root (FIG. 23, B1), ascending dorsal tracts of the spinal cord (FIG. 23, C1), and peripheral nerves (FIG. 23, D1). Typical histopathological findings with the normal counterparts are pictured in FIG. 23, A1-D2; high magnification images of varying stages of DRG pathology are also shown. Early in the degenerative process, the neuronal cell bodies appear relatively normal with only proliferating satellite cells in addition to microglial cells and infiltrating mononuclear cells (neuronophagia, FIG. 23, panel E). As the lesions progress, the neuronal cell bodies exhibit evidence of degeneration (FIG. 23, panel F, vertical arrow) characterized by small, irregular- or angular-shaped cells with fading or absent nuclei and cytoplasmic hypereosinophilia. End-stage neuronal cell body degeneration (FIG. 23, panel G, circles) involves their complete obliteration (FIG. 23, panel G, star) by satellite cells, microglial cells and mononuclear cells. The severity of the histological findings in DRG and corresponding axons is graded based on the percentage of neurons or axons that are affected on an average high-power field: 0 as absence of lesion, 1 as minimal (<10%), 2 mild (10-25%), 3 moderate (25-50%), 4 marked (50-95%), and 5 severe (>95%). DRG represent a mosaic with an abundance of neurons being normal and only a minority of neurons showing degeneration on a given section. We consider spinal cord axonopathy as a better indicator of DRG pathology as it represents the collation of axons coming from all the DRG. We define DRG pathology as histopathological findings within the DRG cell bodies and spinal cord or spinal cord alone throughout this manuscript.

### Studies and Populations Characteristics

We aggregated data from 33 studies including 256 animals injected with AAV vectors or vehicle controls at the Gene Therapy Program at Penn from 2013 to 2020. A summary of these studies is provided in the table below.

TABLE
NHP population and studies characteristics

| Parameter | Number of Animals | | | | | |
|---|---|---|---|---|---|---|
| Species | Rhesus | Rhesus | Rhesus | Cynomolgus | Cynomolgus | Cynomolgus |
| | (all) | (male) | (female) | (all) | (male) | (female) |
| | 237 | 134 | 103 | 19 | 13 | 6 |

| | | |
|---|---|---|
| Sex | Male | Female |
| | 147 | 109 |

| | | | |
|---|---|---|---|
| Age | Infant (1 month) | Juvenile (1-3 years) | Adult (>3 years) |
| | 4 | 58 | 194 |

| | | | | | |
|---|---|---|---|---|---|
| Route of | IV | ICM | LP | IV + ICM | IM |
| Administration** | 27 | 213 | 6 | 4 | 4 |

| Dose Range | IV | ICM and LP |
|---|---|---|
| | 1e13 GC/kg-2e14 GC/kg | 1e12 GC-3e14 GC |
| Capsids | 5 | |
| Promoters | 5 | |
| Transgenes | 20 | |
| Capsid | GLP study | Non-GLP |
| Manufacturing | (column-purified vector) | (iodixanol-purified vector) |
| for ICM/LP | 101 | 103 |
| studies | | |

**Un-injected n = 2 (no pathology)

### Effect of Study Characteristics on Severity of DRG Pathology

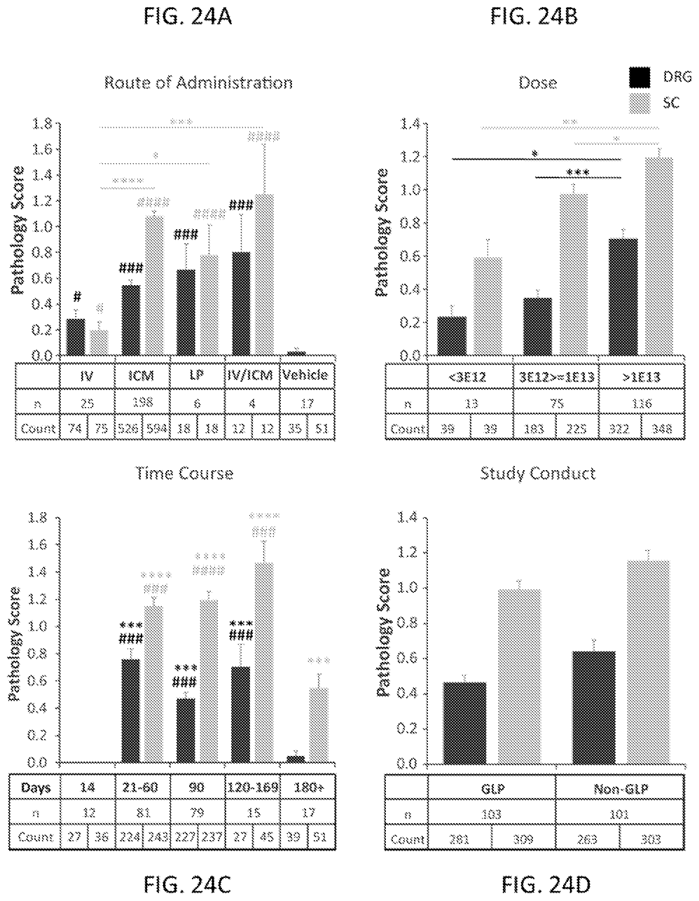

DRG pathology was observed in 83% of NHP that received AAV ICM or LP (170/205 animals), 32% of NHP for the IV route (8/25 animals), 100% for the combination ICM+IV (4/4 animals) and 0% for intramuscular (IM, 0/4 animals). Pathologists graded the DRG lesions based on severity score in DRG and their corresponding axons in spinal cord and peripheral nerves. Scores were obtained for each DRG and spinal region (cervical, thoracic, and lumbar). Severity in DRG was lower than in spinal cord because each spinal cord region groups the totality of axons coming from DRG, thus collating pathology scores from several DRG (FIG. 23). The study design parameters that significantly impacted the severity of the pathology were the route of administration (ROA), dose, and necropsy time point (FIG. 24A—FIG. 24C). The compliance with GLP practice for nonclinical laboratory studies (as set in title 21 of the Code of Federal Regulations, part 58) did not impact the severity of the pathology (FIG. 24D). All the ROAs except IM led to significant pathology in both DRG and spinal cord when compared with vehicle controls (p=0.04 DRG and spinal cord, IV versus vehicle; p<0.001 DRG and p<0.0001 spinal cord, all other routes versus vehicle). ICM, LP, and ICM/IV were all similar and significantly worse than IV (IV vs ICM p<0.0001; IV vs LP p=0.02; IV vs ICM/IV p=0.0006-FIG. 24A). IM (not shown) did not lead to pathology (all score 0) and was similar to vehicle controls. For all the following analyses, we only considered animals with intra-CSF administration (i.e., ICM or LP). The 2 lower dose ranges (<3E+12 GC and 3E+12-1E+13 GC) were similar whereas the maximal dose range (>1E+13 GC) led to significantly worse pathology scores than both the lowest (p=0.009, spinal cord) and the middle dose range (p=0.001 DRG, p=0.05 spinal cord; FIG. 24B). The post-injection time point (i.e., when the necropsy was performed and tissues were analyzed) showed similar pathology severity between 21-60 days, 90 days, and 120-169 days. Pathology was not seen at the early (i.e., day 14) time point and the longer follow-ups greater than or equal to 180 days showed a significant reduction of severity compared to all other time-points (p<0.0001 spinal cord; p<0.0001 DRG D90; p<0.001 DRG other time-points).

### Effect of Animal Characteristics on Severity of DRG Pathology

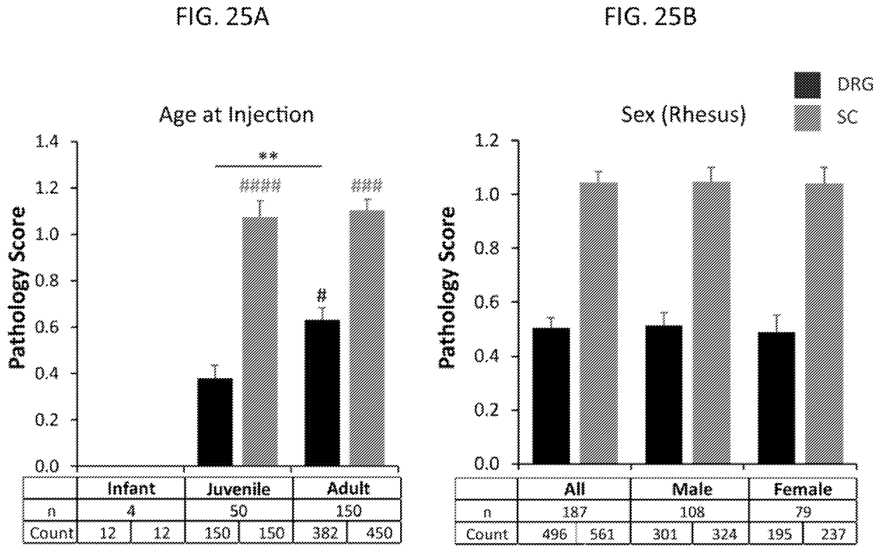

Age at vector administration had a significant effect on pathology severity. Juvenile animals had less severe DRG degeneration when compared with adults (p=0.003) but similar spinal cord axonopathy (FIG. 25A). The 4 animals treated as infants had no sign of DRG or spinal cord pathology as previously reported (J. Hordeaux et al. Hum Gene Ther 30, 957-966, 2019). This result needs to be interpreted with caution due to the small n and possible effect of the study endpoint (4 years post-injection). As shown on FIG. 24A-FIG. 24D, study duration has an impact on the severity of pathology and it is unclear whether the age at injection and/or study duration underpin the absence of pathology. Additionally, and importantly, sex had no effect on SC or DRG pathology (FIG. 24B).

### Effect of Vector Characteristics on Severity of DRG Pathology

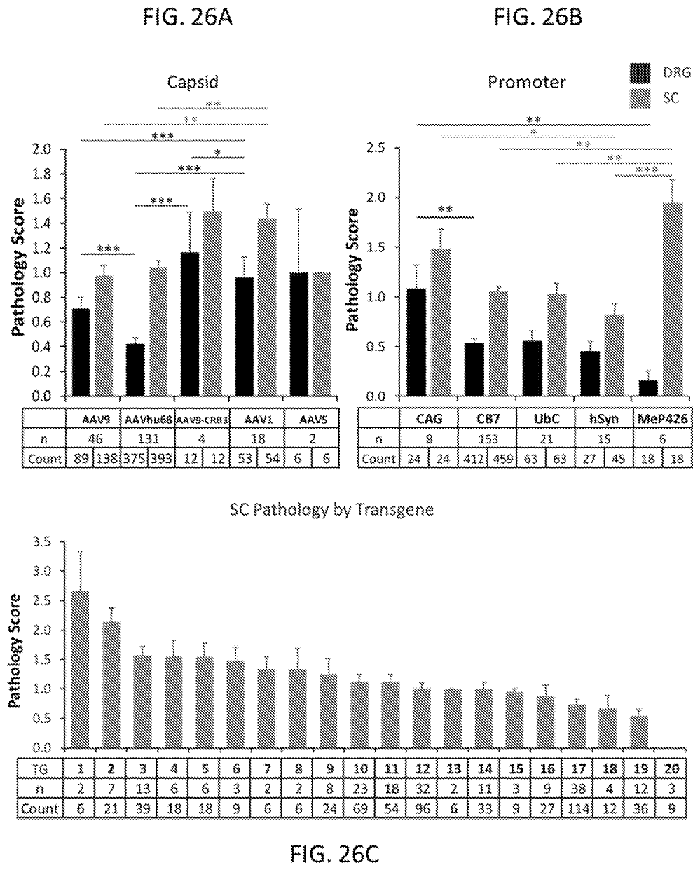

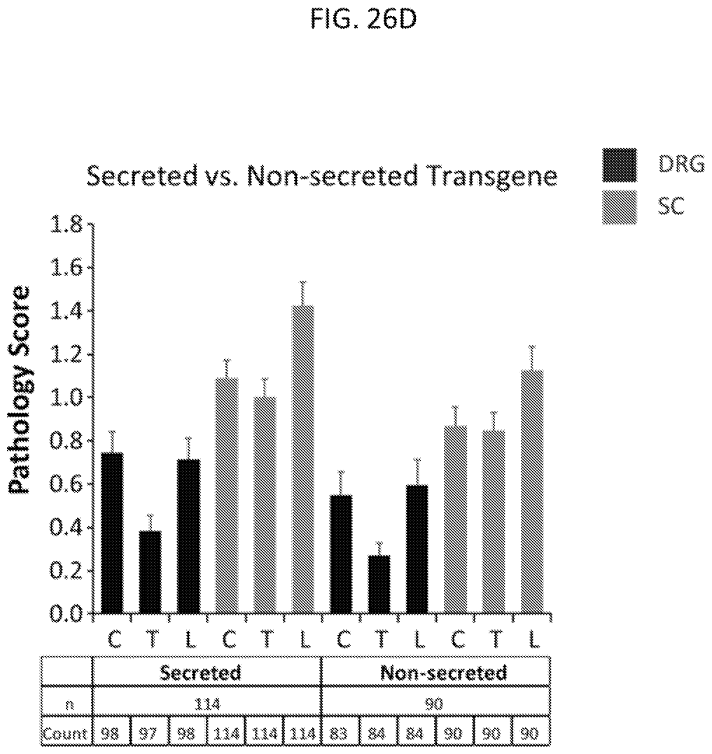

DRG neurodegeneration was present with all capsids, although there were some differences in severity amongst serotypes (FIG. 26A). Such variations, when restricted to DRG and not seen in spinal cord scores, may not be meaningful since the DRG represents a mosaic more susceptible to sampling artifact than the spinal cord cross-sections. Axonopathy and DRG scores were both significantly worse with AAV1 than AAVhu68 (p=0.01 spinal cord; p=0.0004 DRG) and with AAV1 than AAV9 (p=0.007 spinal cord and DRG—FIG. 26A). Ubiquitous promoters CAG, CB7, and UbC were all similar to each other while CAG led to worse axonopathy than hSyn (p=0.028) and MeP426 worse than CB7 (p=0.001), UbC (p=0.002) and hSyn (p=0.0003; FIG. 26B). We tested 20 different transgenes and all but one caused DRG pathology (FIG. 26C). Pathology severity varied greatly between transgenes (from 0.5 to 2.7 average spinal cord axonopathy score); pathology severity was 20-25% less for non-secreted transgenes compared to secreted transgenes (FIG. 26D; for DRG, secreted mean=0.61, non-secreted mean=0.47, p=0.05; for SC, secreted mean=1.17, non-secreted mean=0.94, p=0.02). Moreover, the purification method (i.e., iodixanol in non-GLP studies and column chromatography in GLP studies) did not impact the presence or severity of DRG pathology (FIG. 26D).

### Regional Severity, and Clinical Manifestations of DRG Pathology

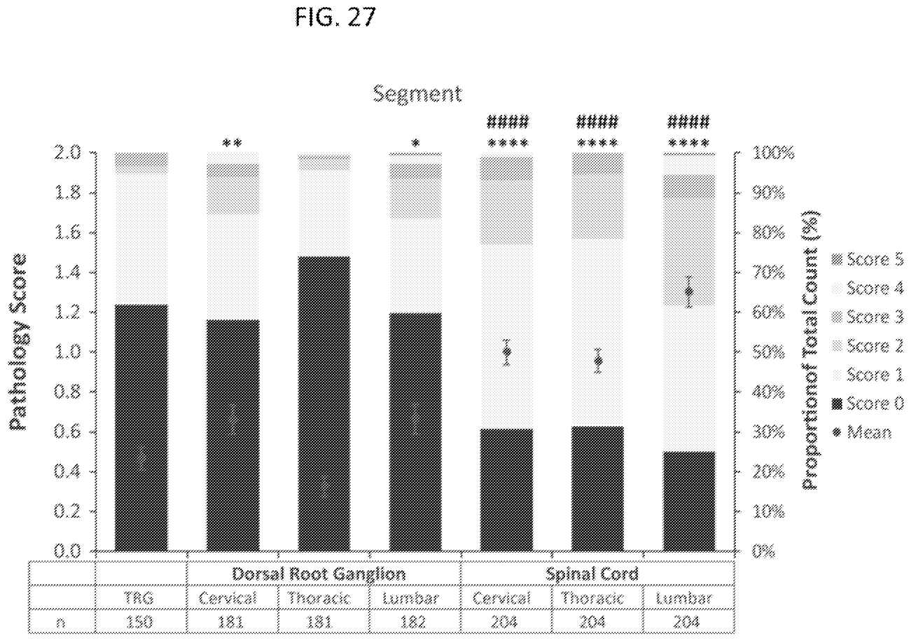

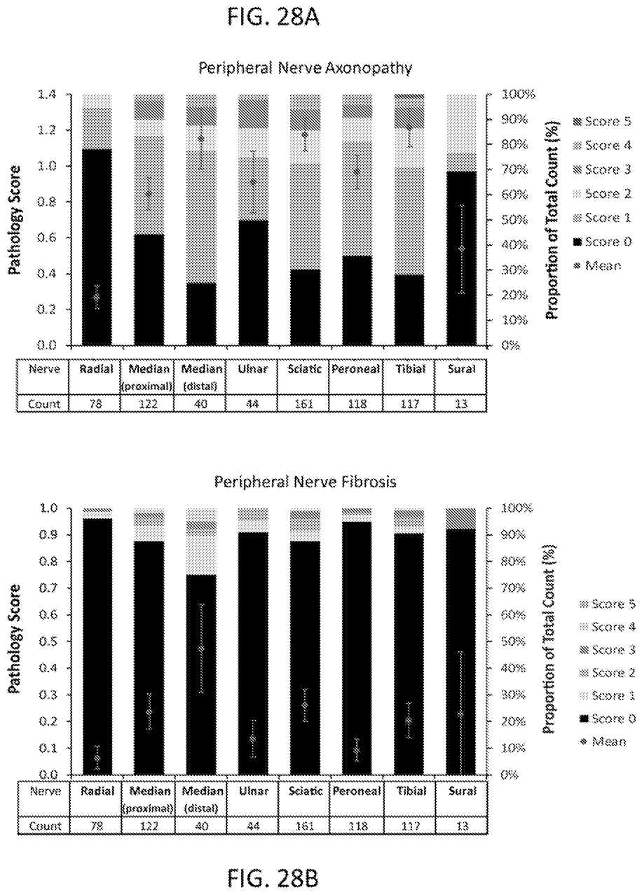

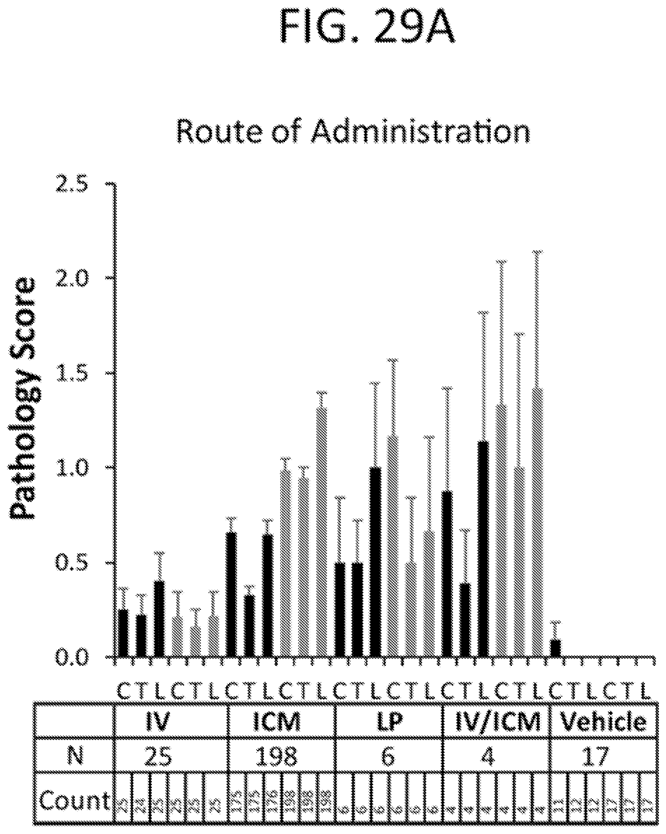

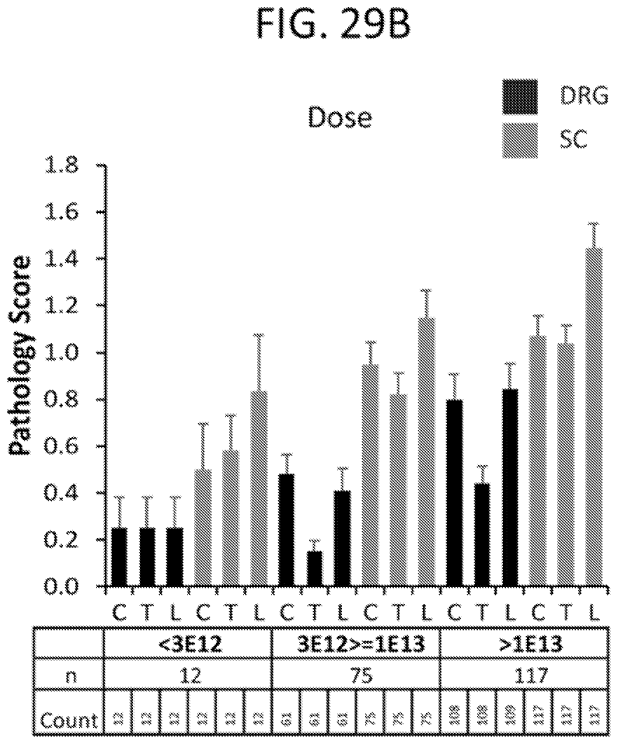

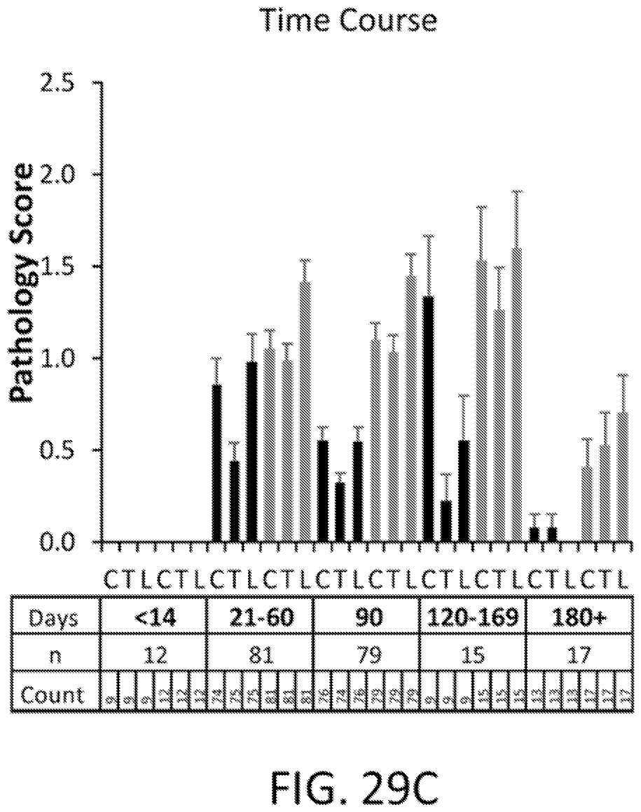

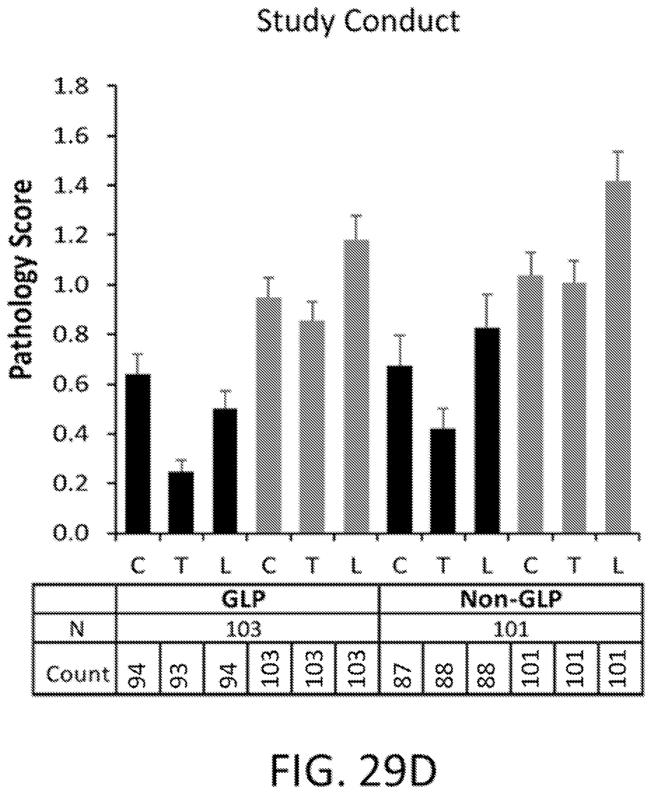

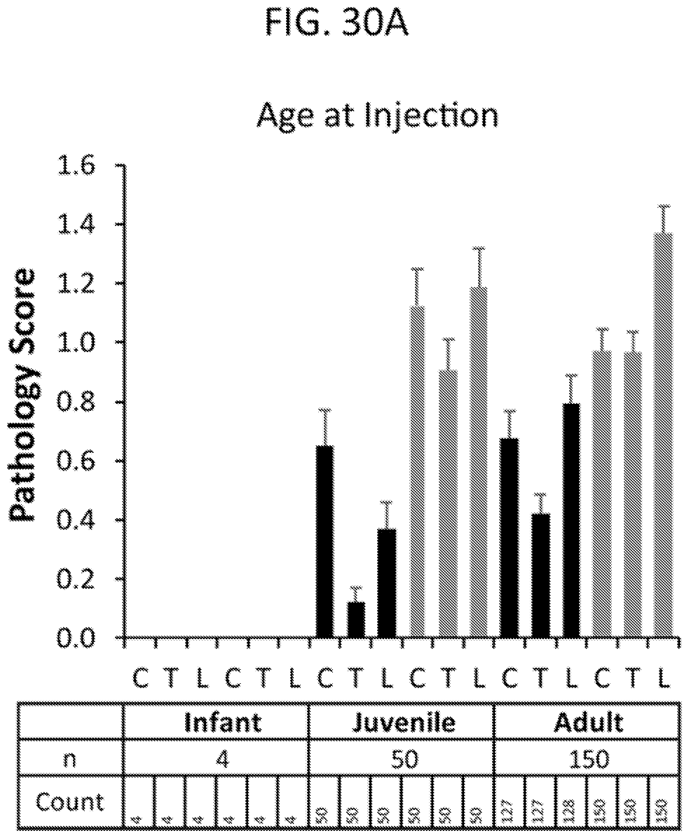

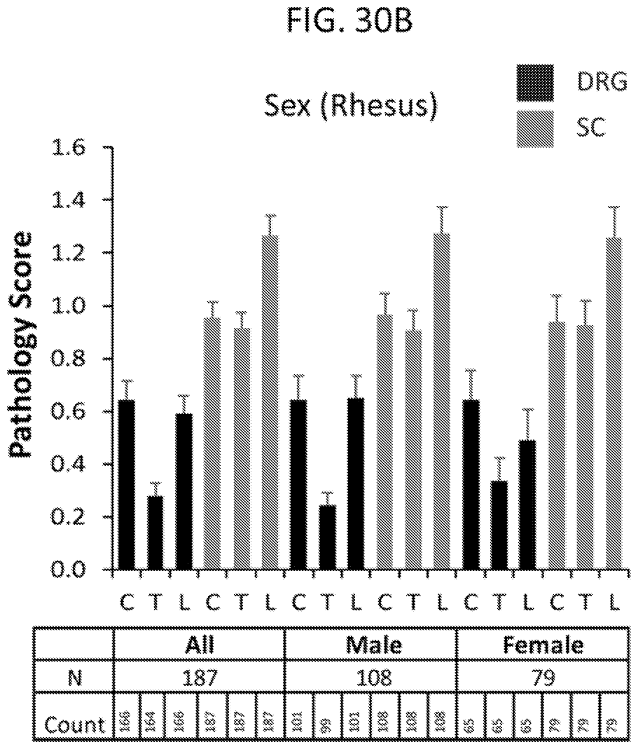

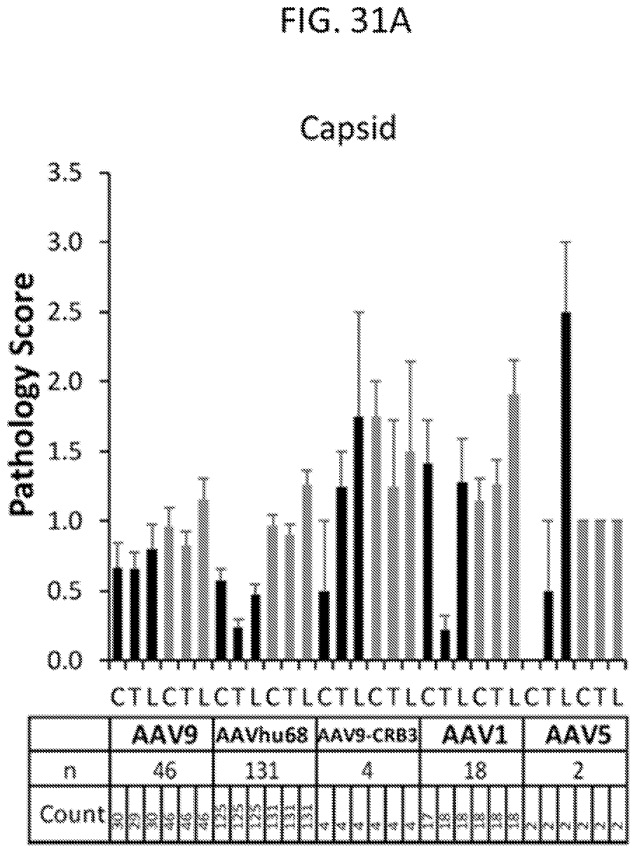

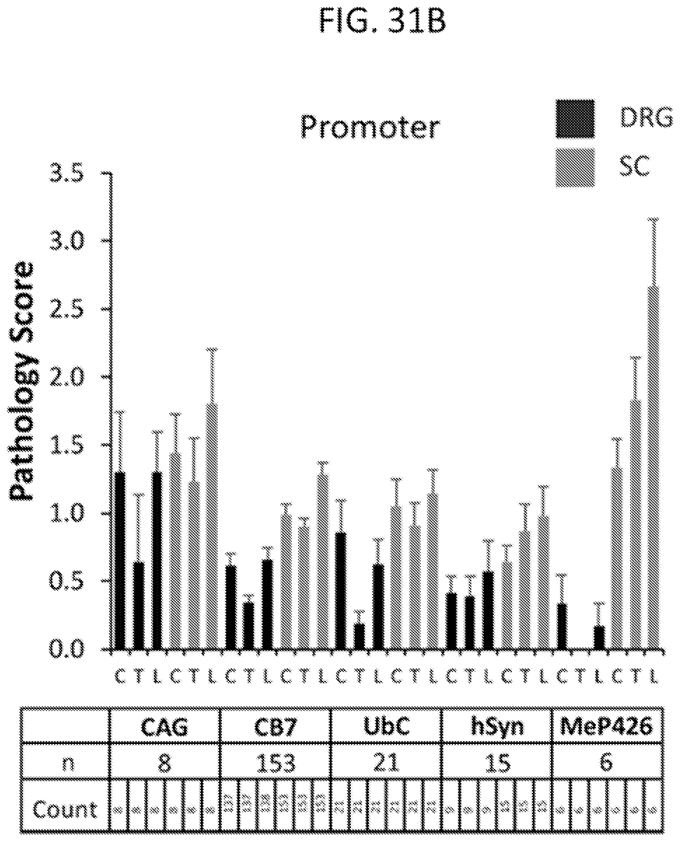

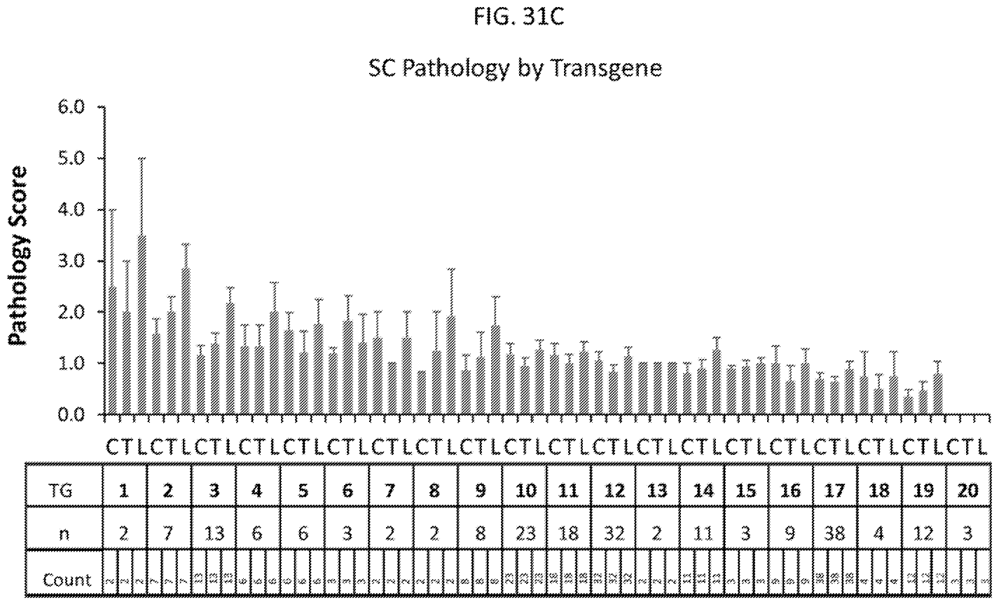

We assessed regional differences in pathology with respect to cervical, thoracic, and lumbar spine. The trigeminal nerve ganglion (TRG) was also analyzed as it represents a sensory ganglion with similar characteristics than the DRG located at the base of the skull inside the subarachnoid space. FIG. 27 shows a distribution of the actual pathology scores in each region as well as the average. TRG pathology was similar than cervical and lumbar DRG (non-significant) whereas the thoracic DRG had a less severe score (p=0.007). SC regional scores were all significantly worse than their corresponding DRG scores (p<0.0001), which is consistent SC collating axons from several DRG, meaning it comprises more lesions. The vast majority of sections have normal or low (grade 1) severity scores, with few grade 4 and very few grade 5 scores reported (grade 5 corresponds to 95% or more of tissue surface affected by the lesion on an average high-power field) (FIG. 27). We performed neurological examination involving cage-side evaluation of mentation, posture, and gait, as well as restrained evaluation of cranial nerves, proprioception, motor strength, sensory function, and reflexes. Of 204 animals administered AAV ICM or LP, only 3 developed obvious pathology with clinical signs of ataxia and/or tremors. All 3 received vectors encoding GFP at doses >1E+13 GC and the pathology appeared 21 days post-injection. Nerve conduction velocities of the median nerve were recorded in 56 animals. Two developed a marked bilateral sensory amplitude reduction at 28 days post-injection that persisted until necropsy. This correlated with marked (grade 4 severity) axonopathy and endoneurial fibrosis in the median nerve but no obvious clinical sequelae. Most animals had low severity grades of axonopathy and fibrosis in peripheral nerves (FIG. 28A and FIG. 28B).

### Discussion

DRG pathology and secondary axonopathy is minimal in the vast majority of our NHP studies and can be challenging to pick up for a non-trained eye. In our first GLP toxicology study evaluating an ICM AAV administration (J. Hordeaux, et al. Mol Ther Methods Clin Dev 10, 79-88, 2018), the CRO who performed the initial pathology assessment missed the lesion which was only caught by a peer-review pathologist experienced in neuropathology. Because neuronal degeneration is sparse and DRG are a mosaic of mostly normal neurons with few degenerative events on a given section, we found that multiple DRG need to be collected for robust histological analysis (we recommend at least 3 per spinal region). An easier way to detect and quantify DRG neuronal damage involves evaluating the secondary consequences of pathology in the cell body by assessing axon degeneration in the spinal cord; this is easier to detect and represents a collation of ascending fibers coming from multiple DRG.

When collecting and carefully analyzing the right tissues, we found some evidence of DRG pathology in 83% of NHP that received AAV ICM and 32% of NHP that received AAV IV. The IV doses showing pathology were as low as 1E+13 GC/kg, a dose currently evaluated in the clinics for several hemophilia trials (B. S. Doshi and V. R. Ther Adv Hematol 9, 273-293, 2018). Manufacturing purification method had no impact on pathology. All capsids and all promoters that we tested demonstrated some level of DRG pathology, which suggests that changing capsid or promoter is not a viable solution. Of relevance for nonclinical study design and for clinical translation, we found that dose and age at injection significantly affected the severity while sex had no impact. The aspect of our studies with the greatest impact on severity of pathology was the transgene, which is consistent with our hypothesis that transgene overexpression drives the early events which lead to degeneration. For most transgenes it was not possible to identify a No Observable Adverse Effect Level (NOAEL) above the Minimum Efficacy Dose (MED).

Time course is important to consider for study design as acute time-points (i.e., day 14 or below) do not show histopathology whereas longer studies (i.e., >180 days) tend to demonstrate less severe pathology, which suggests a lack of progression and possible partial remission over time. Our experience with health authorities has involved incorporating two necropsy time points—one after the onset of pathology (i.e., around 1 month) and another to show the pathology is not getting worse (i.e., 4 to 6 months). The four NHP infants that were dosed at 1-month of age included in our meta-analysis were remarkable for the absence of DRG and SC axon pathology despite good transgene expression levels when the animals were necropsied almost 4 years post-injection (J. Hordeaux et al. Hum Gene Ther 30, 957-966, 2019). This observation may suggest a more favorable safety profile when dosing infants or that acute pathology does not progress and in fact resolves; we did not have any early time point necropsies in this study.

None of the animals that received a therapeutic transgene (i.e., not a reporter gene such as GFP) demonstrated any clinical findings. In later studies, we incorporated routine monitoring of sensory neuron pathology through the use of nerve conduction velocity measurements. We did find NCV abnormalities in two animals associated with more severe peripheral nerves axonopathy and fibrosis (i.e., grade 4 severity) without evidence of clinical sequelae.

In summary, we showed that pathology in DRGs is a consistent finding in virtually all NHPs studies when AAV vector is delivered into the subarachnoid space and in many studies when higher doses are administered systemically. Our meta-analysis is remarkable for a notable absence of any clinical sequelae. Careful analysis of other nonclinical studies in other species failed to show any evidence of DRG pathology, except in newborn pigs, suggesting NHPs are the best model for evaluating this potential pathology. Monitoring of sensory neuropathies in human CNS trials and high dose IV studies seems prudent to determine if clinically meaningful DRG pathology occurs.

### Example 7: Development of a AAVrh91-Mediated MPSI Gene Therapy

Nonclinical studies are performed to evaluate the impact on safety and efficacy of DRG detargeting miRNA target sites on the MPSI transgene delivery. As described in Example 2, a strategy to repress transgene expression in DRG by cloning four tandem repeats of targets for miR183, a DRG enriched miR, in the 3′ UTR region of the expression cassette was effective to ablate GFP expression in DRG while conferring some enhancement of transgene expression elsewhere (brain, liver, heart). When tested in NHP, the four tandem repeats reduced expression in DRG, with 80% reduction of mRNA ISH signal observed in transduced DRG from NHP injected 1×10¹³ GC ICM with AAVhu68.hIDUA-4×miR183 when compared to animals injected with AAVhu68.hIDUA at the same dose. This reduction was enough to prevent DRG pathology and secondary axonopathy completely. The studies described below utilize a capsid with improved tropism and biodistribution in the CNS, AAVrh91 (an AAV1 variant) and/or delivery using an Ommaya reservoir for CNS-targeted administration, as is common for clinical drug administration and sampling.

### Nonclinical Research Studies

### NHP Pilot Study—NextGen DRG and Capsid Comparison

This study is designed to obtain preliminary data on safety, pharmacology, and vector biodistribution after intra cisterna magna (ICM) administration into rhesus macaques.

Study Design:
Vectors:

1. AAVhu68.hIDUAcoV1

2. AAVrh91.hIDUA coV1

3. AAVrh91.hIDUA coV1.4×miR183

4. AAVrh91.hIDUA coV1.4×miR182
Number of animals: 15 (n=3/group)
Route of Administration: ICM
Dose: 3×10¹³ GC
In-life Duration: 90 days

In-life analyses include daily cage side observations, a standardized neurological assessment, periodic bleeds for serum chemistry panels, complete blood counts, coagulation panel, complement activation, liver function tests, periodic CSF taps for CSF chemistry and cell counts. Serum and PBMCs are collected to investigate humoral and cellular immune responses to the capsid and transgene.

Following completion of the in-life phase of this study at 90 days post-vector administration, a full necropsy is performed with tissues harvested for a comprehensive histopathological examination (board-certified veterinary pathologist with peer review) and analysis of vector biodistribution by quantitative PCR and quantification of hIDUA expression. Lymphocytes are harvested from the blood, spleen, liver and deep cervical lymph nodes to examine the presence of CTLs in these organs at the time of necropsy. The vectors with miR targets sequences are expected to best reduce and/or eliminate DRG degeneration and associated axonopathy, while demonstrating optimal biodistribution in key tissues.

NHP Pilot Study—AAVrh91 Vector with ICV Reservoir

A study is performed to evaluate the safety, pharmacology, and vector biodistribution after administration of AAV.GFP via an intraventricular reservoir/catheter system implanted in rhesus macaques. Following the study, the a vector is chosen to be evaluated via this route of administration.

Study Design:
Number of animals: 3
Route of Administration: ICV
Dose: 3×10¹³ GC
In-life Duration: 90 days

In-life analyses include daily cage side observations, a standardized neurological assessment, periodic bleeds for serum chemistry panels, complete blood counts, coagulation panel, complement activation, liver function tests, periodic CSF taps for CSF chemistry and cell counts. Serum and PBMCs are collected to investigate humoral and cellular immune responses to the capsid and transgene.

Following completion of the in-life phase of this study at 90 days post-vector administration, a full necropsy is performed with tissues harvested for a comprehensive histopathological examination (board-certified veterinary pathologist with peer review) and analysis of vector biodistribution by quantitative PCR and quantification of hIDUA expression. Lymphocytes arr harvested from the blood, spleen, liver and deep cervical lymph nodes to examine the presence of CTLs in these organs at the time of necropsy.

### Efficacy and Dose-Range Study in MP SI Mice

AAVrh9lvectors are evaluated to determine hIDUA expression and efficacy compared to the previous MPSI candidates when administered ICV into MPSI mice in a pilot dose-ranging study. Readouts include readouts of serum and liver IDUA activity and storage reduction in the CNS.

Study Design:
Number of animals: —50 (n=10/group, including WT/KO controls)
Route of Administration: ICV
Dose:
1.3×10¹¹ GC
4.5×10¹⁰ GC
1.3×10¹⁰ GC
In-life Duration: 90 days

### Sequence Listing Free Text

The following information is provided for sequences containing free text under numeric identifier <223>

| SEQ ID NO: | |
|---|---|
| (containing | |
| free text) | Free text under <223> |
| 1 | <223> miR-183 target |
| 2 | <223> mirR-96 target |
| 3 | <223> miR-182 target |
| 4 | <223> miR-145 target |
| 5 | <223> spacer (i) |
| 6 | <223> spacer (ii) |
| 7 | <223> spacer (iii) |
| 10 | <223> ITR.CB7.CI.eGFP.miR145.rBG.ITR |
| | <220> |
| | <221> repeat_region |
| | <222> (1) . . . (130) |
| | <223> 5′- AAV2 - ITR |
| | <220> |
| | <221> misc_feature |
| | <222> (1) . . . (130) |
| | <223> 5′- AAV2 - ITR |
| | <220> |
| | <221> promoter |
| | <222> (198) . . . (579) |
| | <223> CMV IE promoter |
| | <220> |
| | <221> promoter |
| | <222> (582) . . . (863) |
| | <223> CB promoter |
| | <220> |
| | <221> misc_feature |
| | <222> (1979) . . . (2698) |
| | <223> eGFP gene |
| | <220> |
| | <221> misc_feature |
| | <222> (2705) . . . (2727) |
| | <223> miR145 |
| | <220> |
| | <221> misc_feature |
| | <222> (2728) . . . (2731) |
| | <223> spacer |
| | <220> |
| | <221> misc_feature |
| | <222> (2732) . . . (2754) |
| | <223> miR145 |
| | <220> |
| | <221> misc_feature |
| | <222> (2755) . . . (2760) |
| | <223> spacer |
| | <220> |
| | <221> misc_feature |
| | <222> (2761) . . . (2783) |
| | <223> miR145 |
| | <220> |
| | <221> misc_feature |
| | <222> (2784) . . . (2789) |
| | <223> spacer |
| | <220> |
| | <221> misc_feature |
| | <222> (2790) . . . (2812) |
| | <223> miR145 |
| | <220> |
| | <221> misc_feature |
| | <222> (2981) . . . (3198) |
| | <223> 3′ITR |
| 11 | <223> ITR.CB7.CI.eGFP.miR182.rGB.ITR |
| | <220> |
| | <221> misc feature |
| | <222> (1) . . . (130) |
| | <223> 5′ITR(AAV2) |
| | <220> |
| | <221> misc_feature |
| | <222> (198) . . . (579) |
| | <223> CMV IE promoter |
| | <220> |
| | <221> misc_feature |
| | <222> (582) . . . (863) |
| | <223> CB promoter |
| | <220> |
| | <221> misc_feature |
| | <222> (958) . . . (1930) |
| | <223> chicken beta-actin intron |
| | <220> |
| | <221> misc_feature |
| | <222> (1979) . . . (2698) |
| | <223> eGFP coding sequence |
| | <220> |
| | <221> misc_feature |
| | <222> (2705) . . . (2728) |
| | <223> miR182 |
| | <220> |
| | <221> misc_feature |
| | <222> (2729) . . . (2732) |
| | <223> spacer |
| | <220> |
| | <221> misc_feature |
| | <222> (2733) . . . (2756) |
| | <223> miR182 |
| | <220> |
| | <221> misc_feature |
| | <222> (2757) . . . (2760) |
| | <223> spacer |
| | <220> |
| | <221> misc_feature |
| | <222> (2763) . . . (2786) |
| | <223> miR182 |
| | <220> |
| | <221> misc_feature |
| | <222> (2787) . . . (2792) |
| | <223> spacer |
| | <220> |
| | <221> misc feature |
| | <222> (2793) . . . (2816) |
| | <223> miR182 |
| | <220> |
| | <221> polyA_signal |
| | <222> (2858) . . . (2984) |
| | <220> |
| | <221> misc_feature |
| | <222> (3073) . . . (3202) |
| | <223> 3′ITR |
| 12 | <223> ITR.CB7.eGFP.miRNA96.rBG.ITR |
| | <220> |
| | <221> misc_feature |
| | <222> (1979) . . . (2699) |
| | <223> eGFP coding sequence |
| | <220> |
| | <221> misc_feature |
| | <222> (2705) . . . (2727) |
| | <223> miR96 |
| | <220> |
| | <221> misc_feature |
| | <222> (2728) . . . (2731) |
| | <223> spacer |
| | <220> |
| | <221> misc_feature |
| | <222> (2732) . . . (2754) |
| | <223> miR96 |
| | <220> |
| | <221> misc_feature |
| | <222> (2755) . . . (2760) |
| | <223> spacer |
| | <220> |
| | <221> misc_feature |
| | <222> (2761) . . . (2783) |
| | <223> miR96 |
| | <220> |
| | <221> misc_feature |
| | <222> (2784) . . . (2789) |
| | <223> spacer |
| | <220> |
| | <221> misc_feature |
| | <222> (2790) . . . (2812) |
| | <223> miR96 |
| 13 | <223> |
| | ITR.CB7.CLeGFP.miRNA183.rBG.ITR |
| | <220> |
| | <221> misc_feature |
| | <222> (1979) . . . (2698) |
| | <223> eGFP coding sequence |
| | <220> |
| | <221> misc_feature |
| | <222> (2705) . . . (2726) |
| | <223> miRNA183 |
| | <220> |
| | <221> misc_feature |
| | <222> (2727) . . . (2730) |
| | <223> spacer |
| | <220> |
| | <221> misc_feature |
| | <222> (2731) . . . (2752) |
| | <223> miRNA183 |
| | <220> |
| | <221> misc_feature |
| | <222> (2753) . . . (2758) |
| | <223> space |
| | <220> |
| | <221> misc_feature |
| | <222> (2781) . . . (2786) |
| | <223> spacer |
| | <220> |
| | <221> misc_feature |
| | <222> (2787) . . . (2808) |
| | <223> miRNA183 |
| 14 | <223> ITR.CB7.CI.hIDUAcoV1.rBG.ITR |
| | <220> |
| | <221> misc_feature |
| | <222> (1) . . . (130) |
| | <223> 5′ITR |
| | <220> |
| | <221> misc_feature |
| | <222> (198) . . . (579) |
| | <223> CMV IE promoter |
| | <220> |
| | <221> misc_feature |
| | <222> (582) . . . (863) |
| | <223> CB promoter |
| | <220> |
| | <221> misc_feature |
| | <222> (836) . . . (839) |
| | <223> TATA |
| | <220> |
| | <221> misc_feature |
| | <222> (958) . . . (1930) |
| | <223> chicken beta-actin intron |
| | <220> |
| | <221> CDS |
| | <222> (1943) . . . (3901) |
| | <223> hIDUAcoVI |
| | <220> |
| | <221> misc_feature |
| | <222> (3908) . . . (3922) |
| | <223> 3UTR insertion site |
| | <220> |
| | <221> misc_feature |
| | <222> (3956) . . . (4082) |
| | <223> Rabbit globin poly A |
| | <220> |
| | <221> misc_feature |
| | <222> (4171) . . . (4300) |
| | <223> 3′ITR |
| 15 | <223> Synthetic Construct |
| 16 | <223> ITR.CB7.CI.hIDUAcoV1.miR183.ITR |
| | <220> |
| | <221> misc_feature |
| | <222> (1) . . . (130) |
| | <223> 5′ITR |
| | <220> |
| | <221> misc_feature |
| | <222> (198) . . . (579) |
| | <223> CMV IE promoter |
| | <220> |
| | <221> misc_feature |
| | <222> (582) . . . (863) |
| | <223> CB promoter |
| | <220> |
| | <221> misc_feature |
| | <222> (836) . . . (839) |
| | <223> TATA |
| | <220> |
| | <221> misc_feature |
| | <222> (958) . . . (1930) |
| | <223> chicken beta-actin intron |
| | <220> |
| | <221> CDS |
| | <222> (1943) . . . (3901) |
| | <220> |
| | <221> misc_feature |
| | <222> (3914) . . . (3935) |
| | <223> miRNA183 |
| | <220> |
| | <221> misc_feature |
| | <222> (3936) . . . (3939) |
| | <223> spacer |
| | <220> |
| | <221> misc_feature |
| | <222> (3940) . . . (3961) |
| | <223> miRNA183 |
| | <220> |
| | <221> misc_feature |
| | <222> (3962) . . . (3967) |
| | <223> spacer |
| | <220> |
| | <221> misc_feature |
| | <222> (3968) . . . (3989) |
| | <223> miRNA183 |
| | <220> |
| | <221> misc_feature |
| | <222> (3990) . . . (3995) |
| | <223> spacer |
| | <220> |
| | <221> misc_feature |
| | <222> (3996) . . . (4017) |
| | <223> miRNA183 |
| | <220> |
| | <221> misc_feature |
| | <222> (4059) . . . (4185) |
| | <223> Rabbit globin poly A |
| | <220> |
| | <221> misc_feature |
| | <222> (4274) . . . (4403) |
| | <223> 3′ITR |
| 17 | <223> Synthetic Construct |
| 22 | <223> engineered sequence |
| 23 | <223> engineered sequence |
| 24 | <223> engineered sequence |
| 25 | <223> engineered sequence |
| 26 | <223> engineered sequence |
| 29 | <223> Synthetic Construct |

All publications cited in this specification are incorporated herein by reference in their entireties. U.S. Provisional Patent Application No. 63/043,600, filed Jun. 24, 2020, U.S. Provisional Patent Application No. 63/038,514, filed Jun. 12, 2020, U.S. Provisional Patent Application No. 63/023,602, filed May 12, 2020, US Provisional Patent Application No. 63/005,894, filed Apr. 6, 2020, U.S. Provisional Patent Application No. 62/972,404, filed Feb. 10, 2020, International Patent Application No. PCT/US19/67872, filed Dec. 20, 2019, U.S. Provisional Patent Application No. 62/934,915, filed Nov. 13, 2019, U.S. Provisional Patent Application No. 62/924,970, filed Oct. 23, 2019, U.S. Provisional Patent Application No. 63/023,593, filed May 12, 2020, U.S. Provisional Patent Application No. 63/038,488, filed Jun. 12, 2020, U.S. Provisional Patent Application No. 63/043,562, filed Jun. 24, 2020, and U.S. Provisional Patent Application No. 63/079,299, filed Sep. 16, 2020, are hereby incorporated by reference in their entireties. Similarly, the SEQ ID NOs which are referenced herein and which appear in the appended Sequence Listing labeled “20-9316PCT.txt” are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) comprising an AAV capsid having packaged therein a vector genome, wherein the vector genome comprises a coding sequence for a functional human alpha-L-iduronidase (hIDUA) and regulatory sequences which direct expression of the hIDUA in a cell, wherein the coding sequence comprises
a) nucleotides 82 to 1959 of SEQ ID NO: 22, or a sequence at least 95% identical thereto;
b) nucleotides 82 to 1959 of SEQ ID NO: 23, or a sequence at least 95% identical thereto;
c) nucleotides 82 to 1959 of SEQ ID NO: 24, or a sequence at least 95% identical thereto;
d) nucleotides 82 to 1959 of SEQ ID NO: 25, or a sequence at least 95% identical thereto; or
e) nucleotides 82 to 1959 of SEQ ID NO: 26, or a sequence at least 95% identical thereto.

2. The rAAV according to claim 1, wherein the hIDUA comprises at least amino acids 28 to 653 of SEQ ID NO: 21, or a sequence at least 95% identical thereto.

3. The rAAV according to claim 1, wherein the hIDUA comprises the native signal peptide.

4. The rAAV according to claim 1, wherein the hIDUA comprises the full-length (amino acids 1 to 653) of SEQ ID NO: 21, or a sequence at least 95% identical thereto.

5. (canceled)

6. The rAAV according to claim 1, wherein the hIDUA comprises a heterologous signal peptide.

7. (canceled)

8. The rAAV according to claim 1, wherein the vector genome further comprises at least one dorsal root ganglion (drg)-specific miRNA target sequence specific for at least one of miR-183, miR-182, or miR-96, the at least one target sequence being operably linked to the 3′ end of the hIDUA coding sequence.

9. The rAAV according to claim 1, wherein the vector genome further comprises an miRNA target sequence selected from
(a)
(SEQ ID NO: 1)
AGTGAATTCTACCAGTGCCATA;
(b)
(SEQ ID NO: 2)
AGCAAAAATGTGCTAGTGCCAAA;
(c)
(SEQ ID NO: 3)
AGTGTGAGTTCTACCATTGCCAAA;
and
(d)
(SEQ ID NO: 4)
AGGGATTCCTGGGAAAACTGGAC.

10. The rAAV according to claim 1, wherein the vector genome further comprises at two, at least three, or at least four drg-specific miRNA target sequences.

11. The rAAV according to claim 1, wherein the AAV capsid is an AAV9 capsid, an AAVhu68 capsid, or an AAVrh91 capsid.

12. An expression cassette comprising a nucleic acid sequence encoding a functional human alpha-galactosidase A (hIDUA) and regulatory sequences that direct expression of the hIDUA in a cell containing the expression cassette, wherein coding sequence comprises
a) nucleotides 82 to 1959 of SEQ ID NO: 22, or a sequence at least 95% identical thereto;
b) nucleotides 82 to 1959 of SEQ ID NO: 23, or a sequence at least 95% identical thereto;
c) nucleotides 82 to 1959 of SEQ ID NO: 24, or a sequence at least 95% identical thereto;
d) nucleotides 82 to 1959 of SEQ ID NO: 25, or a sequence at least 95% identical thereto; or
e) nucleotides 82 to 1959 of SEQ ID NO: 26, or a sequence at least 95% identical thereto.

13. The expression cassette according to claim 12, wherein the hIDUA comprises at least amino acids 28 to 653 of SEQ ID NO: 21, or a sequence at least 95% identical thereto.

14. The expression cassette according to claim 12, wherein the hIDUA comprises the native signal peptide.

15. The expression cassette according to claim 12, wherein the hIDUA comprises the full-length (amino acids 1 to 653) of SEQ ID NO: 21, or a sequence at least 95% identical thereto.

16. (canceled)

17. The expression cassette according to claim 12 or 13, wherein the hIDUA comprises a heterologous signal peptide.

18. (canceled)

19. The expression cassette according to claim 12, wherein the expression cassette further comprises at least one dorsal root ganglion (drg)-specific miRNA target sequence specific for at least one of miR-183, miR-182, or miR-96, the at least one target sequence being operably linked to the 3′ end of the hIDUA coding sequence.

20. The expression cassette according to claim 12, wherein the expression cassette further comprises an miRNA target sequence selected from
(a)
(SEQ ID NO: 1)
AGTGAATTCTACCAGTGCCATA;
(b)
(SEQ ID NO: 2)
AGCAAAAATGTGCTAGTGCCAAA;
(c)
(SEQ ID NO: 3)
AGTGTGAGTTCTACCATTGCCAAA;
and
(d)
(SEQ ID NO: 4)
AGGGATTCCTGGGAAAACTGGAC.

21.-24. (canceled)

25. A recombinant nucleic acid comprising a sequence encoding a functional hIDUA, wherein the coding sequence comprises nucleotides 82 to 1959 of SEQ ID NO: 22, 23, 24, 25, or 26, or a sequence at least 95% identical thereto, or wherein the coding sequence comprises nucleotides 1 to 1959 of SEQ ID NO: 22, 23, 24, 25, or 26, or a sequence at least 95% identical thereto.

26.-27. (canceled)

28. A host cell comprising the recombinant nucleic acid according to claim 25.

29. A pharmaceutical composition comprising the rAAV according to claim 1, and a pharmaceutically-acceptable carrier.

30. A method of treating a subject diagnosed with mucopolysaccharidosis type I (MPS I), said method comprising administering to the subject the pharmaceutical composition according to claim 29.

36.-36. (canceled)


SEQUENCE LISTING (29 sequences)

SEQ ID NO: 1 - DNA, 22 bp, Artificial Sequence
  miR-183 target
agtgaattctaccagtgccata

SEQ ID NO: 2 - DNA, 23 bp, Artificial sequence
  mirR-96 target
agcaaaaatgtgctagtgccaaa

SEQ ID NO: 3 - DNA, 24 bp, Artificial sequence
  miR-182 target
agtgtgagttctaccattgccaaa

SEQ ID NO: 4 - DNA, 23 bp, Artificial sequence
  miR-145 target
agggattcctgggaaaactggac

SEQ ID NO: 5 - DNA, 4 bp, Artificial sequence
  Spacer (i)
ggat

SEQ ID NO: 6 - DNA, 6 bp, Artificial sequence
  Spacer (ii)
cacgtg

SEQ ID NO: 7 - DNA, 6 bp, Artificial sequence
  spacer iii
gcatgc

SEQ ID NO: 8 - DNA, 2211 bp, adeno-associated virus hu68
  CDS (1)..(2211)
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 9 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - DNA, 3198 bp, Artificial sequence
  ITR.CB7.CI.eGFP.miR145.rBG.ITR
  repeat_region (1)..(130): 5′ - AAV2 - ITR
  misc_feature (1)..(130): 5′ - AAV2 - ITR
  promoter (198)..(579): CMV IE promoter
  promoter (582)..(863): CB promoter
  misc_feature (1979)..(2698): eGFP gene
  misc_feature (2705)..(2727): miR145
  misc_feature (2728)..(2731): spacer
  misc_feature (2732)..(2754): miR145
  misc_feature (2755)..(2760): spacer
  misc_feature (2761)..(2783): miR145
  misc_feature (2784)..(2789): spacer
  misc_feature (2790)..(2812): miR145
  misc_feature (2981)..(3198): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagggattcctgggaaaactggacggatagggattcctgggaaaactggaccacgtgagggattcctgggaaaactggacgcatgcagggattcctgggaaaactggacgcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 3202 bp, Artificial sequence
  ITR.CB7.CI.eGFP.miR182.rGB.ITR
  misc_feature (1)..(130): 5′ ITR (AAV2)
  misc_feature (198)..(579): CMV IE promoter
  misc_feature (582)..(863): CB promoter
  misc_feature (958)..(1930): chicken beta-actin intron
  misc_feature (1979)..(2698): eGFP coding sequence
  misc_feature (2705)..(2728): miR182
  misc_feature (2729)..(2732): spacer
  misc_feature (2733)..(2756): miR182
  misc_feature (2757)..(2760): spacer
  misc_feature (2763)..(2786): miR182
  misc_feature (2787)..(2792): spacer
  misc_feature (2793)..(2816): miR182
  polyA_signal (2858)..(2984)
  misc_feature (3073)..(3202): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagtgtgagttctaccattgccaaaggatagtgtgagttctaccattgccaaacacgtgagtgtgagttctaccattgccaaagcatgcagtgtgagttctaccattgccaaagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 12 - DNA, 3198 bp, Artificial sequence
  ITR.CB7.eGFP.miRNA96.rBG.ITR
  misc_feature (1979)..(2699): eGFP coding sequence
  misc_feature (2705)..(2727): miR96
  misc_feature (2728)..(2731): spacer
  misc_feature (2732)..(2754): miR96
  misc_feature (2755)..(2760): spacer
  misc_feature (2761)..(2783): miR96
  misc_feature (2784)..(2789): spacer
  misc_feature (2790)..(2812): miR96
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagcaaaaatgtgctagtgccaaaggatagcaaaaatgtgctagtgccaaacacgtgagcaaaaatgtgctagtgccaaagcatgcagcaaaaatgtgctagtgccaaagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 13 - DNA, 3194 bp, Artificial sequence
  ITR.CB7.CI.eGFP.miRNA183.rBG.ITR
  misc_feature (1979)..(2698): eGFP coding sequence
  misc_feature (2705)..(2726): miRNA183
  misc_feature (2727)..(2730): spacer
  misc_feature (2731)..(2752): miRNA183
  misc_feature (2753)..(2758): spacer
  misc_feature (2781)..(2786): spacer
  misc_feature (2787)..(2808): miRNA183
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattacttaatacgactcactataggctagtaatacgactcactatagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggtaccagtgaattctaccagtgccataggatagtgaattctaccagtgccatacacgtgagtgaattctaccagtgccatagcatgcagtgaattctaccagtgccatagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 4300 bp, Artificial sequence
  ITR.CB7.CI.hIDUAcoV1.rBG.ITR
  misc_feature (1)..(130): 5′ ITR
  misc_feature (198)..(579): CMV IE promoter
  misc_feature (582)..(863): CB promoter
  misc_feature (836)..(839): TATA
  misc_feature (958)..(1930): chicken beta-actin intron
  CDS (1943)..(3901): hIDUAcoV1
  misc_feature (3908)..(3922): 3UTR insertion site
  misc_feature (3956)..(4082): Rabbit globin poly A
  misc_feature (4171)..(4300): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagccaccatgaggcctctcagacctagagctgctctgctggcactgctggcttctctgcttgctgctcctcctgtggctcctgccgaagctcctcatctggtgcacgtggatgccgccagagcactgtggcccctgagaagattttggcggagcaccggcttttgccctccactgcctcattctcaggccgaccagtacgtgctgagctgggaccagcaactgaacctggcctacgtgggagccgtgcctcacagaggcattaagcaagtgcggacccactggctgctggaactggtcacaacaagaggcagcacaggcagaggcctgagctacaacttcacccacctggacggctacctggacctgctgagagagaatcagctgctgcctggcttcgagctgatgggctctgcctctggccacttcaccgacttcgaggacaagcagcaggttttcgagtggaaggacctggtgtccagcctggccagacggtacatcggcagatacggactggcccacgtgtccaagtggaacttcgagacctggaacgagcccgaccaccacgacttcgacaacgtgtcaatgaccatgcagggctttctgaactactacgacgcctgcagcgagggcctgagagctgcttctcctgctctgagacttggcggccctggcgactcttttcacacccctccaagaagccctctgtcctggggactgctgagacactgtcacgacggcaccaatttcttcaccggcgaggctggcgtgcggctggattatatcagcctgcacagaaagggcgccagaagcagcatcagcatcctggaacaagagaaggtggtggcccagcagatcagacagctgttccccaagttcgccgacacacccatctacaacgacgaggccgatcctctcgttggctggtcacttcctcagccttggagagccgatgtgacctatgccgccatggtggtcaaagtgatcgcccagcaccagaatctgctgctcgccaataccaccagcgcctttccatacgctctgctgagcaacgacaacgccttcctgagctatcaccctcatcctttcgctcagcggaccctgaccgccagattccaagtgaacaacacccggcctccacacgtgcagctgctgagaaaaccagtgctgacagccatgggcctgctcgccctgctggacgaagaacaactgtgggccgaagtgtcccaggccggaacagtgctggatagcaatcacacagtgggcgtgctggcctccgctcatagacctcaaggaccagccgatgcttggagggctgccgtgctgatctacgccagcgacgatacaagggctcaccccaacagatccgtggccgtgacactgagactgagaggcgttccaccaggacctggcctggtgtacgtgaccagatacctggacaacggcctgtgcagccctgatggcgaatggcgtagactaggcagacctgtgtttcctaccgccgagcagttcagacggatgagagccgctgaagatcccgtggctgctgctccaagacctcttccagctggtggcagactgactctgaggcctgcactcagactgcctagtctgctgctggtgcacgtctgtgccagacctgagaagcctcctggccaagtgacaagactgagggccctgccactgacacagggacagctggttcttgtttggagcgacgagcacgtgggcagcaagtgtctgtggacctacgagatccagttcagccaggacggcaaggcctacacacccgtgtctagaaagcctagcaccttcaacctgttcgtgttcagccccgatacaggcgccgtgtctggcagctatagagtcagagccctggactactgggccagaccaggaccattttctgaccccgtgccttacctggaagtgcccgttcctagaggccctccttctcctggaaatccctgataaggtaccatacctaggctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 15 - protein, 653 aa, Artificial sequence
  Synthetic Construct
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 16 - DNA, 4403 bp, Artificial sequence
  ITR.CB7.CI.hIDUAcoV1.miR183.ITR
  misc_feature (1)..(130): 5′ ITR
  misc_feature (198)..(579): CMV IE promoter
  misc_feature (582)..(863): CB promoter
  misc_feature (836)..(839): TATA
  misc_feature (958)..(1930): chicken beta-actin intron
  CDS (1943)..(3901)
  misc_feature (3914)..(3935): miRNA183
  misc_feature (3936)..(3939): spacer
  misc_feature (3940)..(3961): miRNA183
  misc_feature (3962)..(3967): spacer
  misc_feature (3968)..(3989): miRNA183
  misc_feature (3990)..(3995): spacer
  misc_feature (3996)..(4017): miRNA183
  misc_feature (4059)..(4185): Rabbit globin poly A
  misc_feature (4274)..(4403): 3′ ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcagccaccatgaggcctctcagacctagagctgctctgctggcactgctggcttctctgcttgctgctcctcctgtggctcctgccgaagctcctcatctggtgcacgtggatgccgccagagcactgtggcccctgagaagattttggcggagcaccggcttttgccctccactgcctcattctcaggccgaccagtacgtgctgagctgggaccagcaactgaacctggcctacgtgggagccgtgcctcacagaggcattaagcaagtgcggacccactggctgctggaactggtcacaacaagaggcagcacaggcagaggcctgagctacaacttcacccacctggacggctacctggacctgctgagagagaatcagctgctgcctggcttcgagctgatgggctctgcctctggccacttcaccgacttcgaggacaagcagcaggttttcgagtggaaggacctggtgtccagcctggccagacggtacatcggcagatacggactggcccacgtgtccaagtggaacttcgagacctggaacgagcccgaccaccacgacttcgacaacgtgtcaatgaccatgcagggctttctgaactactacgacgcctgcagcgagggcctgagagctgcttctcctgctctgagacttggcggccctggcgactcttttcacacccctccaagaagccctctgtcctggggactgctgagacactgtcacgacggcaccaatttcttcaccggcgaggctggcgtgcggctggattatatcagcctgcacagaaagggcgccagaagcagcatcagcatcctggaacaagagaaggtggtggcccagcagatcagacagctgttccccaagttcgccgacacacccatctacaacgacgaggccgatcctctcgttggctggtcacttcctcagccttggagagccgatgtgacctatgccgccatggtggtcaaagtgatcgcccagcaccagaatctgctgctcgccaataccaccagcgcctttccatacgctctgctgagcaacgacaacgccttcctgagctatcaccctcatcctttcgctcagcggaccctgaccgccagattccaagtgaacaacacccggcctccacacgtgcagctgctgagaaaaccagtgctgacagccatgggcctgctcgccctgctggacgaagaacaactgtgggccgaagtgtcccaggccggaacagtgctggatagcaatcacacagtgggcgtgctggcctccgctcatagacctcaaggaccagccgatgcttggagggctgccgtgctgatctacgccagcgacgatacaagggctcaccccaacagatccgtggccgtgacactgagactgagaggcgttccaccaggacctggcctggtgtacgtgaccagatacctggacaacggcctgtgcagccctgatggcgaatggcgtagactaggcagacctgtgtttcctaccgccgagcagttcagacggatgagagccgctgaagatcccgtggctgctgctccaagacctcttccagctggtggcagactgactctgaggcctgcactcagactgcctagtctgctgctggtgcacgtctgtgccagacctgagaagcctcctggccaagtgacaagactgagggccctgccactgacacagggacagctggttcttgtttggagcgacgagcacgtgggcagcaagtgtctgtggacctacgagatccagttcagccaggacggcaaggcctacacacccgtgtctagaaagcctagcaccttcaacctgttcgtgttcagccccgatacaggcgccgtgtctggcagctatagagtcagagccctggactactgggccagaccaggaccattttctgaccccgtgccttacctggaagtgcccgttcctagaggccctccttctcctggaaatccctgataaggtaccagtgaattctaccagtgccataggatagtgaattctaccagtgccatacacgtgagtgaattctaccagtgccatagcatgcagtgaattctaccagtgccatagcggccgcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 17 - protein, 653 aa, Artificial sequence; identical to SEQ ID NO: 15
  Synthetic Construct

SEQ ID NO: 18 - DNA, 2211 bp, adeno-associated virus 9
  CDS (1)..(2211)
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 19 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 20 - DNA, 1962 bp, Homo sapiens
  CDS (1)..(1962)
atgcgtcccctgcgcccccgcgccgcgctgctggcgctcctggcctcgctcctggccgcgcccccggtggccccggccgaggccccgcacctggtgcatgtggacgcggcccgcgcgctgtggcccctgcggcgcttctggaggagcacaggcttctgccccccgctgccacacagccaggctgaccagtacgtcctcagctgggaccagcagctcaacctcgcctatgtgggcgccgtccctcaccgcggcatcaagcaggtccggacccactggctgctggagcttgtcaccaccagggggtccactggacggggcctgagctacaacttcacccacctggacgggtacctggaccttctcagggagaaccagctcctcccagggtttgagctgatgggcagcgcctcgggccacttcactgactttgaggacaagcagcaggtgtttgagtggaaggacttggtctccagcctggccaggagatacatcggtaggtacggactggcgcatgtttccaagtggaacttcgagacgtggaatgagccagaccaccacgactttgacaacgtctccatgaccatgcaaggcttcctgaactactacgatgcctgctcggagggtctgcgcgccgccagccccgccctgcggctgggaggccccggcgactccttccacaccccaccgcgatccccgctgagctggggcctcctgcgccactgccacgacggtaccaacttcttcactggggaggcgggcgtgcggctggactacatctccctccacaggaagggtgcgcgcagctccatctccatcctggagcaggagaaggtcgtcgcgcagcagatccggcagctcttccccaagttcgcggacacccccatttacaacgacgaggcggacccgctggtgggctggtccctgccacagccgtggagggcggacgtgacctacgcggccatggtggtgaaggtcatcgcgcagcatcagaacctgctactggccaacaccacctccgccttcccctacgcgctcctgagcaacgacaatgccttcctgagctaccacccgcaccccttcgcgcagcgcacgctcaccgcgcgcttccaggtcaacaacacccgcccgccgcacgtgcagctgttgcgcaagccggtgctcacggccatggggctgctggcgctgctggatgaggagcagctctgggccgaagtgtcgcaggccgggaccgtcctggacagcaaccacacggtgggcgtcctggccagcgcccaccgcccccagggcccggccgacgcctggcgcgccgcggtgctgatctacgcgagcgacgacacccgcgcccaccccaaccgcagcgtcgcggtgaccctgcggctgcgcggggtgccccccggcccgggcctggtctacgtcacgcgctacctggacaacgggctctgcagccccgacggcgagtggcggcgcctgggccggcccgtcttccccacggcagagcagttccggcgcatgcgcgcggctgaggacccggtggccgcggcgccccgccccttacccgccggcggccgcctgaccctgcgccccgcgctgcggctgccgtcgcttttgctggtgcacgtgtgtgcgcgccccgagaagccgcccgggcaggtcacgcggctccgcgccctgcccctgacccaagggcagctggttctggtctggtcggatgaacacgtgggctccaagtgcctgtggacatacgagatccagttctctcaggacggtaaggcgtacaccccggtcagcaggaagccatcgaccttcaacctctttgtgttcagcccagacacaggtgctgtctctggctcctaccgagttcgagccctggactactgggcccgaccaggccccttctcggaccctgtgccgtacctggaggtccctgtgccaagagggcccccatccccgggcaatccatga

SEQ ID NO: 21 - protein, 653 aa, Homo sapiens; identical to SEQ ID NO: 15

SEQ ID NO: 22 - DNA, 1959 bp, Artificial Sequence
  engineered sequence
atgaggcctctcagacctagagctgctctgctggcactgctggcttctctgcttgctgctcctcctgtggctcctgccgaagctcctcatctggtgcacgtggatgccgccagagcactgtggcccctgagaagattttggcggagcaccggcttttgccctccactgcctcattctcaggccgaccagtacgtgctgagctgggaccagcaactgaacctggcctacgtgggagccgtgcctcacagaggcattaagcaagtgcggacccactggctgctggaactggtcacaacaagaggcagcacaggcagaggcctgagctacaacttcacccacctggacggctacctggacctgctgagagagaatcagctgctgcctggcttcgagctgatgggctctgcctctggccacttcaccgacttcgaggacaagcagcaggttttcgagtggaaggacctggtgtccagcctggccagacggtacatcggcagatacggactggcccacgtgtccaagtggaacttcgagacctggaacgagcccgaccaccacgacttcgacaacgtgtcaatgaccatgcagggctttctgaactactacgacgcctgcagcgagggcctgagagctgcttctcctgctctgagacttggcggccctggcgactcttttcacacccctccaagaagccctctgtcctggggactgctgagacactgtcacgacggcaccaatttcttcaccggcgaggctggcgtgcggctggattatatcagcctgcacagaaagggcgccagaagcagcatcagcatcctggaacaagagaaggtggtggcccagcagatcagacagctgttccccaagttcgccgacacacccatctacaacgacgaggccgatcctctcgttggctggtcacttcctcagccttggagagccgatgtgacctatgccgccatggtggtcaaagtgatcgcccagcaccagaatctgctgctcgccaataccaccagcgcctttccatacgctctgctgagcaacgacaacgccttcctgagctatcaccctcatcctttcgctcagcggaccctgaccgccagattccaagtgaacaacacccggcctccacacgtgcagctgctgagaaaaccagtgctgacagccatgggcctgctcgccctgctggacgaagaacaactgtgggccgaagtgtcccaggccggaacagtgctggatagcaatcacacagtgggcgtgctggcctccgctcatagacctcaaggaccagccgatgcttggagggctgccgtgctgatctacgccagcgacgatacaagggctcaccccaacagatccgtggccgtgacactgagactgagaggcgttccaccaggacctggcctggtgtacgtgaccagatacctggacaacggcctgtgcagccctgatggcgaatggcgtagactaggcagacctgtgtttcctaccgccgagcagttcagacggatgagagccgctgaagatcccgtggctgctgctccaagacctcttccagctggtggcagactgactctgaggcctgcactcagactgcctagtctgctgctggtgcacgtctgtgccagacctgagaagcctcctggccaagtgacaagactgagggccctgccactgacacagggacagctggttcttgtttggagcgacgagcacgtgggcagcaagtgtctgtggacctacgagatccagttcagccaggacggcaaggcctacacacccgtgtctagaaagcctagcaccttcaacctgttcgtgttcagccccgatacaggcgccgtgtctggcagctatagagtcagagccctggactactgggccagaccaggaccattttctgaccccgtgccttacctggaagtgcccgttcctagaggccctccttctcctggaaatccc

SEQ ID NO: 23 - DNA, 1959 bp, Artificial Sequence
  engineered sequence
atgaggcctctcagacctagagctgctctgctggcactgctggcttctctgcttgctgctcctcctgtggctcctgccgaagctcctcatctggtgcacgtggatgccgccagagcactgtggcccctgagaagattttggcggagcaccggcttttgccctccactgcctcattctcaagccgaccaatacgtgctgagctgggaccagcaactgaacctggcctacgtgggagccgtgcctcacagaggcattaagcaagtgcggacccactggctgctggaactggtcacaacaagaggcagcacaggcagaggcctgagctacaacttcacccacctggacggctacctggacctgctgagagagaatcagctgctgcctggcttcgagctgatgggctctgcctctggccacttcaccgacttcgaggacaagcagcaggttttcgagtggaaggacctggtgtccagcctggccagacgctacatcggcagatacggactggcccacgtgtccaagtggaacttcgagacctggaacgagcccgaccaccacgacttcgacaacgtgtcaatgaccatgcagggctttctgaactactacgacgcctgcagcgagggcctgagagctgcttctcctgctctgagacttggcggccctggcgactcttttcacacccctccaagaagccctctgtcctggggactgctgagacactgtcacgacggcaccaatttcttcaccggcgaggctggcgtgcggctggattatatcagcctgcacagaaagggcgccagaagcagcatcagcatcctggaacaagagaaggtggtggcccagcagatcagacagctgttccccaagttcgccgacacacccatctacaacgacgaggccgatcctctcgttggctggtcacttcctcagccttggagagccgatgtgacctatgccgccatggtggtcaaagtgatcgcccagcaccagaatctgctgctcgccaataccaccagcgcctttccatacgctctgctgagcaacgacaacgccttcctgagctatcaccctcatcctttcgctcagcggaccctgaccgccagattccaagtgaacaacacccggcctccacacgtgcagctgctgagaaaaccagtgctgacagccatgggcctgctcgccctgctggacgaagaacaactgtgggccgaagtgtcccaggccggaacagtgctggatagcaatcacacagtgggcgtgctggcctccgctcatcgacctcaaggaccagccgatgcttggagggctgccgtgctgatctacgccagcgacgatacaagggctcaccccaacagatccgtggccgtgacactgagactgagaggcgttccaccaggacctggcctggtgtacgtgaccagatacctggacaacggcctgtgcagccctgatggcgaatggcgtagactaggcagacctgtgtttcctaccgccgagcagttcagacggatgagagccgctgaagatcccgtggctgctgctccaagacctcttccagctggtggcagactgactctgaggcctgcactcagactgcctagtctgctgctggtgcacgtctgtgccagacctgagaagcctcctggccaagtgacaagactgagggccctgccactgacacagggacagctggttcttgtttggagcgacgagcacgtgggcagcaagtgtctgtggacctacgagatccagttcagccaggacggcaaggcctacacacccgtgtctagaaagcctagcaccttcaacctgttcgtgttcagccccgatacaggcgccgtgtctggcagctatagagtcagagccctggactactgggccagaccaggaccattttctgaccccgtgccttacctggaagtgcccgttcctagaggccctccttctcctggaaatccc

SEQ ID NO: 24 - DNA, 1959 bp, Artificial Sequence
  engineered sequence
atgcgacccttgcgaccaagagccgccctgcttgcgttgttggcttcccttttggctgcaccacccgtcgccccggcagaggcgcctcacctcgtgcatgtagacgcagcccgcgccctctggccattgcggcgattctggaggtctaccggtttctgcccacccctgcctcattcacaagccgaccaatacgttttgtcctgggatcaacagctcaaccttgcgtatgtaggcgctgttccacaccggggaattaagcaggtccggactcactggcttcttgagttggttacgacgaggggttcaacagggagagggttgtcctacaactttacacatctggatggttatttagacctgctccgagaaaaccaacttctccctggattcgagctcatgggctctgcctctggtcactttacggatttcgaggataaacagcaagtcttcgagtggaaggatcttgtgagcagccttgccagaagatatataggaagatacgggttggcacacgtatctaaatggaactttgagacttggaacgaacccgaccatcacgactttgacaacgtatctatgacgatgcaaggcttcctcaactattatgacgcgtgcagtgaaggtctgagggctgcgtctccggcgctcagattgggaggtcctggagacagctttcatacgcctccgcgatcccctcttagctgggggttgctcagacattgccacgacggtacaaacttcttcaccggcgaggcaggtgttaggctcgactacatctccctgcaccgaaagggcgcgcgatcttctatcagtatattggaacaagagaaagttgtggctcaacaaattcgccaactttttcccaaattcgctgataccccgatttacaacgacgaagccgacccattggtaggttggagtcttccccagccttggcgagcggatgtcacctacgcagctatggtagttaaagtgattgcgcaacaccaaaatctccttcttgccaacacgacttctgcgtttccatacgcgttgttgtctaacgacaacgcctttttgtcctatcacccgcacccgtttgcgcagcgaactctgaccgcaagatttcaggttaacaatactcggccaccacacgtacagcttttgcgcaaacctgtattgactgccatgggattgttggcactgttggatgaagaacaactgtgggctgaagtgagccaggcgggtacagttctggacagtaatcacactgtaggcgtgctcgccagtgctcaccgaccacaggggccggcagacgcctggagagctgctgtactcatctacgcatcagacgataccagggcgcatcccaatagatccgtcgccgtaactcttcggcttcggggcgtcccgccagggccgggccttgtttatgttactcgatacttagacaacggactttgtagtcctgatggtgaatggcgtagattagggcggcctgtctttcctactgcggagcagttcagacgaatgagggctgctgaggacccagttgcagctgcaccccgcccgcttccggccggtggcagacttacgctcaggcccgcgcttaggttgccgtccttgttgcttgtccacgtttgcgctcgcccagaaaagccgccgggacaggttacacgacttcgggctctgcccctgacgcagggacaactggtgcttgtttggagtgatgaacacgtaggaagcaagtgcttgtggacttacgagatacaattcagccaagacggcaaggcttatacccctgtttcacgcaaaccttctacttttaatttgtttgtcttttctccggatacgggggcggtctctgggtcatatcgcgtgagagcactcgattactgggctagaccagggccatttagcgatcccgttccttatctggaggtgcccgtcccgaggggtccaccaagccccggaaatccg

SEQ ID NO: 25 - DNA, 1959 bp, Artificial Sequence
  engineered sequence
atgaggcccctgaggcccagggccgccctgctggccctgctggcctccctgctggccgctcctcccgtggccccagccgaggctcctcacctggtgcacgtggacgccgccagggccctgtggcccctgaggaggttctggaggtccaccggcttctgtcctcctctgccccactcccaagccgaccaatacgtgctgtcctgggaccagcagctgaacctggcctatgtgggagctgtgccccacaggggcatcaagcaagtgaggacccactggctgctggagctggtgaccaccaggggctccaccggcaggggcctgtcctacaacttcacccacctggacggctacctggacctgctgagggagaaccagctgctgcccggcttcgagctgatgggctccgcctccggccacttcaccgacttcgaggacaagcagcaggtgttcgagtggaaggacctggtgtcctccctggccaggagatacatcggcagatacggcctggcccacgtgtccaagtggaacttcgagacctggaacgagcccgaccaccacgacttcgacaacgtgtccatgaccatgcagggcttcctgaactactacgacgcctgctccgagggcctgagggccgcctctcctgccctgaggctgggaggacctggcgactccttccacacacctccaaggtctcccctgtcctggggcctgctgaggcactgccacgacggcaccaacttcttcaccggagaggctggagtgaggctggactacatctccctgcaccggaagggcgccaggtcctccatctccatcctggagcaggagaaggtggtggcccagcagatcaggcagctgttccccaagttcgccgacacacctatctacaacgacgaggccgatcctctggtgggctggtccctgccccagccctggagggctgatgtgacctatgctgccatggtggtgaaggtgatcgcccagcaccagaacctgctgctggccaacaccacctccgccttcccctacgccctgctgtccaacgacaacgccttcctgtcctaccatcctcaccccttcgcccaaaggaccctgaccgccaggttccaagtgaacaacaccaggcctcctcacgtgcagctgctgaggaagcccgtgctgaccgccatgggcctgctggccctgctggacgaggagcagctgtgggccgaggtgtcccaggccggcaccgtgctggactccaaccacaccgtgggcgtgctggcctccgcccaccggccccagggccccgccgacgcctggagggccgccgtgctgatctacgcctccgacgacaccagggcccaccccaacaggtccgtggccgtgaccctgaggctgaggggcgtgcctcccggccccggcctggtgtacgtgaccaggtatctggacaacggcctgtgctctcctgacggcgagtggaggaggctgggcaggcccgtgttccccaccgccgagcagttcaggaggatgagggccgccgaggaccccgtggccgccgctcctcgacccctgcctgctggaggcaggctgaccctgaggcccgccctgaggctgccctccctgctgctggtgcacgtgtgcgccaggcccgagaagcctcctggccaggtgaccaggctgagggccctgcccctgacccagggccagctggtgctggtgtggtccgacgagcacgtgggctccaagtgcctgtggacctacgagatccagttctcccaggacggcaaggcctacacacctgtgtccaggaagccctccaccttcaacctgttcgtgttctctcctgacaccggcgccgtgtccggctcctaccgagtgagggccctggactactgggccaggcccggccccttctccgaccccgtgccctacctggaggtgcccgtgcccaggggacctccttctcctggcaacccc

SEQ ID NO: 26 - DNA, 1959 bp, Artificial Sequence
  engineered sequence
atgagacctttaagaccacgtgctgctctgctggctttactcgcttctttactggccgctcctcccgtggcccccgctgaagctcctcacttagtgcacgtggatgccgccagagctctgtggcctctgaggagattttggaggtccactggtttctgccctcctttaccccatagccaagctgatcagtacgtgctgagctgggatcagcaactgaatttagcctacgtcggcgctgtgcctcacagaggcattaagcaagtgaggacccactggctgctggaactggtcaccactcgtggcagcactggtagaggactcagctacaatttcacacatttagacggctatctggatttactgagagagaatcagttattacccggtttcgagctcatgggaagcgcctccggccatttcaccgacttcgaggacaagcagcaagttttcgaatggaaagatttagtgtcctccctcgctcgtaggtacatcggaagatacggtttagcccacgtcagcaagtggaacttcgagacttggaatgagcccgatcaccacgattttgacaatgtcagcatgaccatgcaaggttttttaaactactacgatgcttgtagcgaaggcctcagagctgccagccccgctctgagactcggcggacccggtgactccttccacacacctcctagaagccctttaagctggggtttactgagacactgtcacgacggcaccaacttctttaccggcgaggccggcgttcgtctcgactatatctctttacatcgtaagggcgctcgttcctccatttccatcctcgaacaagaaaaggtggtggctcagcagattaggcaactcttccccaagttcgccgacacccctatctataatgacgaggctgatcctctggtcggctggtctttaccccaaccttggagagctgatgtcacctacgctgccatggtggtgaaggtgatcgcccagcaccagaatttattattagctaacaccacatccgccttcccttacgctctgctgtccaacgataacgccttcctcagctatcaccctcacccctttgcccagagaactttaaccgctagattccaagttaataacaccagacccccccacgtccagttattacgtaagcccgttctgacagccatgggtttactcgctttactggacgaagagcagctgtgggctgaagtgagccaagctggcaccgtgctggatagcaaccacacagtgggcgtgctcgccagcgctcataggcctcaaggacccgctgatgcttggagggctgccgtgctgatctacgccagcgacgacacaagggctcaccccaataggtccgtggctgttacactgagactcagaggcgtcccccccggtcccggtttagtgtatgtgaccagatatttagacaacggactgtgctcccccgacggagagtggagaagactgggtcgtcccgtgtttcctaccgccgagcagtttaggaggatgagagctgccgaggatcccgttgccgccgccccacgtcctttacccgccggcggaaggctgacattaagacccgctttaagactgccctctttactgctcgtgcatgtgtgtgccagacccgaaaagcctcccggacaagttaccagactgagagccctccctctgacccaaggtcagctggtgctggtgtggtccgacgaacacgtgggcagcaagtgtttatggacctatgagatccagttctcccaagatggcaaagcttacacccccgtgtctcgtaaacctagcaccttcaatttattcgtgtttagccccgacaccggagccgtgtccggcagctatcgtgtgagagctttagactactgggctaggcccggcccttttagcgatcccgtgccttatttagaagtgcccgttcccagaggaccccccagccccggaaatcct

SEQ ID NO: 27 - DNA, 2211 bp, adeno-associated virus rh.91
  CDS (1)..(2211)
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 28 - protein, 736 aa, adeno-associated virus rh.91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 29 - DNA, 2211 bp, Artificial Sequence
  synthetic construct
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa